(19) United States
(12) Patent Application Publication
(10) Pub. No.: US 2008/0286312 A1
(43) Pub. Date: Nov. 20, 2008

(54) Membrane-anchored beta2 microglobulincovalently linked to MHC class I peptide epitopes

(75) Inventors: Gideon Gross, M.P. Korazim (IL); Alon Margalit, M.P. Western Galilee (IL)

Correspondence Address: BROWDY AND NEIMARK, P.L.L.C.;624 NINTH STREET, NW, SUITE 300, WASHINGTON, DC, 20001-5303, US

(73) Assignee: Gavish-Galilee Bio Applications Ltd., Kiryat Shmona (IL)

(21) Appl. No.: 11/541,566

(22) Filed: Oct. 3, 2006

Related U.S. Application Data
(63) Continuation-in-part of application No. 10/517,784, filed on Dec. 13, 2004.
(60) Provisional application No. 60/388,273, filed on Jun. 12, 2002.

(51) Int. Cl. A61K 39/00; C07H 21/00; C12N 15/63; A61P 37/02; C12N 5/00

(52) U.S. Cl. 424/277.1; 536/23.5; 435/320.1; 435/325; 424/184.1

(57) ABSTRACT

The invention provides a polynucleotide comprising a sequence encoding a polypeptide that is capable of high level presentation of antigenic peptides on antigen-presenting cells, wherein the polypeptide comprises a β2-microglobulin molecule that is linked through its carboxyl terminal to a bridge peptide which spans the whole distance to the cell membrane, said bridge peptide being linked to a polypeptide stretch consisting of the full or partial transmembrane and/or cytoplasmic domains selected from the group consisting of a toll-like receptor (TLR) polypeptide, a CD40 polypeptide, and TLR and CD40 polypeptides fused in tandem, that allows the anchorage of the β2-microglobulin molecule to the cell membrane, and through its amino terminal to at least one antigenic peptide comprising an MHC class I epitope, wherein said antigenic peptide is preferably derived from a tumor-associated antigen or from a pathogenic antigen. Antigen presenting cells and DNA and cellular vaccines for treatment of cancer and infectious diseases, are also provided.

### CROSS-REFERENCE TO RELATED APPLICATIONS

The present application is a continuation-in-part application of U.S. patent application Ser. No. 10/517,784, filed Jun. 12, 2003, and claims the benefit under 35 U.S.C. §365(c) of international application No. PCT/IL03/00501, filed Jun. 12, 2003, and claims the benefit of U.S. Provisional Patent Application No. 60/388,273, filed Jun. 12, 2002, now expired, the entire contents of each and all these applications being herewith incorporated by reference in their entirety as if fully disclosed herein.

### FIELD OF THE INVENTION

The present invention is in the field of Immunology and relates to DNA molecules encoding chimeric polypeptides comprising β₂-microglobulin and a polypeptide stretch for anchoring the β₂-microglobulin molecule to the cell membrane, herein referred to as single-chimeric β₂-microglobulin (scβ₂m), and to such DNA molecules further comprising at least one antigenic peptide linked to the amino terminal of the β₂-microglobulin molecule, herein referred to as double-chimeric β₂-microglobulin (dcβ₂m), and to antigen-presenting cells expressing said scβ₂m and dcβ₂m polypeptides, as novel tools for efficient CTL induction for the treatment of cancer and infectious diseases, or in the prevention and/or treatment of T-cell mediated disorders and conditions such as graft rejection and autoimmune diseases.

Abbreviations: APC: antigen-presenting cell; β₂m: β₂-microglobulin; BCR: B cell receptor; CDR: complementarity-determining region; CTL: cytotoxic T lymphocyte; dcβ₂m: double-chimeric β₂-microglobulin; DC: dendritic cells; ER: endoplasmic reticulum; GPI: glycosyl-phosphatidylinositol; Ha: hemagglutinin; hβ₂m: human β₂-microglobulin; HLA: human leukocyte antigen (=human MHC); Ig: immunoglobulin; ITAM: immunoreceptor tyrosine-based activation motif; mAb: monoclonal antibody; mβ₂m: mouse β₂-microglobulin; MFI: mean fluorescence intensity; MHC: major histocompatibility complex; NP: nucleoprotein; OVA: chicken ovalbumin; RT-PCR: reverse transcriptase-polymerase chain reaction; scβ₂m: single-chimeric β₂-microglobulin TAA: tumor-associated antigen; TAP: transporter associated with antigen processing; TCR: T-cell receptor; T_{H}: T helper cells; TLR: toll-like receptor; Treg: regulatory T cells TRP: tyrosinase-related protein.

### BACKGROUND OF THE INVENTION

The discovery, in recent years, of tumor-associated antigens (TAAs) in a growing list of primary human tumors has led to the recognition that most, if not all types of human cancers express tumor antigens. The realization that some TAAs can elicit immune responses that lead to tumor rejection, has refueled interest in the field of cancer immunology, raising hopes for the development of potent anticancer immunotherapeutic tools and cancer vaccines (for reviews, see Minev et al., 1999; Gilboa et al., 1998; Rosenberg, 1999).

Tumor antigens can be divided according to the type of immune response they induce: humoral or cellular, which can be further subdivided into CD4⁺ (helper) and CD8⁺ (cytotoxic) T cell responses. Most TAAs known today were identified by their ability to induce cellular responses, predominantly by cytotoxic T lymphocytes (CTLs). CTLs utilize their clonotypic T cell receptor (TCR) to recognize antigenic peptides presented on major histocompatibility complex (MHC) class I molecules, which are expressed by most nucleated cells in the body. These proteins consist of a membrane-attached α heavy chain, which harbors three structurally distinct extracellular domains (α1-α3), and a non-covalently associated β₂ microglobulin (β₂m) light chain, that is not anchored to the cell membrane. Peptides, typically 8-10 amino acids long, bind to a special groove formed between the two membrane-distal domains of the α chain, α1 and α2, mainly via 2-3 dominant anchor residues.

CTLs serve as the major effector arm of the immune system and represent an important component of an animal's or an individual's immune response against a variety of pathogens and cancers. CTLs which have been specifically activated against a particular antigen are capable of killing the cell that contains or expresses the antigen. CTLs are particularly important in providing an effective immune response against intracellular pathogens, such as a wide variety of viruses, and some bacteria and parasites, as well as against tumors.

Some tumors down-regulate MHC class I expression, implying a strong selective pressure imposed by CTLs. In addition, CTLs are capable of recognizing single amino acid substitutions such as those that occur in TAAs resulting from point mutations. All these suggest that TAAs-derived MHC class I peptides are likely to constitute effective rejection antigens.

CTL activation, or priming, requires that antigenic peptides be presented initially on professional antigen-presenting cells (APCs), primarily dendritic cells (DCs), in secondary lymphoid organs (Steinman, 1989). In addition to highly efficient antigen presentation, DCs provide co-stimulatory signals, which are mandatory for T cell priming, usually by engagement of their up-regulated B7 molecules with their CD28 receptor on the T cell (Janeway and Bottomly, 1994). Acquisition of the ability of the DC to prime CTLs is primarily mediated by antigen-specific CD4 T cells in a process referred to as ‘licensing’. It involves interaction of the TCR of the CD4 T cell with an antigenic peptide on an MHC class II molecule on the DC and concomitant engagement of the CD40 ligand (CD40L) on the T cell with the DC CD40 receptor (Guermonprez et al., 2002). Another unique feature of DCs is their ability to present peptides generated from exogenous proteins on their MHC class I molecules, a phenomenon generally referred to as cross-presentation (Heath an Carbone, 2001). Indeed, it is due to these unique properties, that autologous DCs are considered ideal for the induction of antitumor responses (for reviews, see Gilboa et al., 1998; Nouri-Shirazi et al., 2000; Chen et al., 2000; Porgador et al., 1996) and are thus widely explored as potential cancer vaccines.

Bone marrow (BM)-derived DC precursors migrate from the blood to various tissues and acquire an immature DC phenotype. These are capable of capturing invading pathogens by using both receptor-mediated and non-mediated pathways. In addition, they become exquisitely sensitive sensors for infection. This is achieved by the expression of a panel of unique receptors, which recognize conserved microbial molecular motifs, known as ‘pathogen-associated molecular patterns’. Prominent among these are the toll-like receptors (TLRs). The particular TLR members engaged at the DC surface polarize the ensuing adaptive response towards the Th1, Th2 or the regulatory T cells (Treg) course (Pulendran, 2004). A Th1 type of response and subsequent CTL induction is mediated by engagement of TLRs 3,4,5,7,8 and 9, which trigger the production of IL-12 (p70) and interferon α, in addition to other cytokines. The appreciation that distinct TLRs can modulate CTL response has had a tremendous impact on the development of adjuvants for cancer vaccines. Among these, different formulations of CpG DNA, dsRNA (or its poly IC analog) and bacterial lipopolysaccharides are extensively investigated as agonists for TLR9, TLR3 and TLR4, respectively. Of particular importance are the recent observations that TLR activation of DCs is necessary for blocking Treg activity (Pasare et al., 2003) and that persistence of this TLR signaling is a prerequisite for achieving this effect and for eliciting an effective CTL response (Yang et al., 2004). Human TLR4 devoid of its ectodomain was shown to confer a constitutively active phenotype on transfected APCs (Medzhitov et al., 1997) and provides an excellent genetic device for triggering both DC maturation and persistent signaling. This has been recently demonstrated with an RNA encoding dominant positive TLR4, which was applied in conjunction with an additional RNA encoding a melanoma TAA (Cisco et al., 2004).

Attempts to develop novel approaches for the generation of cancer vaccines have taken two major routes. One makes use of the complete antigenic repertoire of the tumor cells. This is accomplished by induction of T cells by irradiated tumor cells, genetically modified to express cytokines, co-stimulatory molecules or foreign MHC, by pulsing of DCs with tumor-derived heat shock proteins, whole tumor cell extracts or total RNA (a minute amount of which can easily be amplified) and fusion of DCs with tumor cells (Zhang et al., 1997; Gong et al., 1997; Gong et al., 2000). These strategies are applicable to many types of tumors and, in theory, can induce a wide spectrum of antitumor CTLs. However, presentation of TAA-derived peptides of potential clinical benefit is not enriched and these protocols may thus fail to induce therapeutic CTLs (Sogn, 2000; Dalgleish, 2001). Furthermore, these procedures do not allow attribution of clinical response to particular antigens and, therefore, useful information cannot be deduced for broader implementation.

The second approach for the generation of cancer vaccines is based on known TAAs. These include the design of peptide, DNA and recombinant viral vaccines, charging DCs with either purified tumor-associated proteins or TAA-derived peptides and presentation of TAA-derived peptides, which are produced following gene delivery into autologous or syngeneic (in mice) DCs (for review, see Gilboa et al., 1998).

Choosing particular HLA-binding peptides allows stringent and reproducible formulations for treatment protocols and rational improvement of immunogenicity by generating heteroclitic peptides of higher HLA affinity. Indeed, as summarized in a recent perspective (Rosenberg et al., 2004), restricting tumor-specific CTL response to only one or few TAA-derived epitopes is the strategy of choice in the majority of clinical vaccine studies. This is primarily implemented by immunizing with synthetic peptides, either alone or loaded onto autologous DCs. However, in contrast to the documented efficacy of peptide-based vaccines in experimental mouse models, the objective clinical response rate in patients with metastatic cancers is less than 3%. The same pattern emerges in clinical studies examining tumor cell lysates, tumor RNA or viral vectors encoding intact TAAs. In addition to the obvious requirement for elevated and prolonged presentation of immunogenic peptides by DCs, the same perspective reiterates the needs for suppressing CD4+CD25+ Tregs and augmenting CTL activation and survival by developing better adjuvants.

Indeed, some encouraging data showing CTL induction and vaccine efficacy came from animal studies exploring either type of above-described approaches. However, clinical success in human trials has so far been limited, with little correlation between the observed number of specific anti-tumor CTLs and the actual clinical response (Sogn, 1998; Moingeon, 2001; Jager et al., 2002). This is attributed, in part, to requirement for help from CD4⁺ cells and to immunosuppressing cytokines produced by the tumor cells, but also to the fact that many of the identified MHC class I-associated TAA peptides are poorly presented on the cell surface because of low level of protein expression and low affinity for their restricting MHC class I molecule (Watson et al., 1995; Vora et al., 1997).

Intracellular proteins, as well as soluble protein antigens delivered into the cytoplasm of a cell, are degraded into short peptides by a cytosolic proteolytic system present in all cells. Those proteins targeted for proteolysis often have a small protein, called ubiquitin, attached covalently to a lysine-amino group near the amino terminal of the protein. These ubiquitin-protein complexes are degraded into a variety of peptides by a multifunctional protease complex called proteasome. Experimental evidence indicates that the immune system utilizes this general pathway of protein degradation to produce small peptides for presentation with class I MHC molecules. The peptides, generated in the cytosol by the proteasome, are translocated by a transporter protein, called TAP (for “transporter associated with antigen processing”), into the endoplasmic reticulum (ER), by a process that requires the hydrolysis of ATP. Within the ER membrane, newly synthesized class I α chain associates with calnexin until β₂m binds to the a chain. The class I a chain-β₂m heterodimer then binds to calreticulin and the TAP-associated protein tapasin. When a peptide delivered by TAP is bound to the class I molecule, folding of MHC class I is complete and it is released from the ER and transported to the surface of the cell. TAP has the highest affinity for peptides containing 8-13 amino acids. Peptides longer than the size required for MHC class I binding are further trimmed in the ER by assigned amino peptidases to acquire the optimal length.

A single cell can display thousands of different MHC class I bound peptides, most of them only at low frequency of less than 0.1% of the total. The density of MHC/peptide complexes on the cell surface determines the degree of T cell responsiveness (Levitsky et al., 1996; Tsomides et al., 1994; Gervois et al., 1996). CTL priming by a professional APC generally requires a higher density of specific complexes than that required on the surface of the target cell for activation of an armed effector CTL (Armstrong et al., 1998; Reis e Souza, 2001). The ability to generate high numbers of particular MHC class I/peptide complexes on the APC itself could, therefore, be of great value for elicitation of strong CTL responses, which may be effective against TAA-derived peptides of an otherwise limited distribution.

This realization has prompted attempts to enhance level of peptide presentation by APCs, either by increasing the intrinsic affinity of the peptide for the restricting MHC class I molecule, or by manipulations aiming at elevating the actual number of specific classI/peptide complexes on the cell surface. A recent study (Tirosh et al., 1999) has examined the effect of peptide affinity on CTL response it elicits, either by a chemical modification, which renders peptide binding to the class I groove irreversible, or by optimizing the MHC anchor residues of the peptide. Working with the TAP-deficient RMA-S cells, it was shown that improving the affinity of a murine TAA-derived peptide could indeed result in significant enhancement of CTL induction and inhibition of tumor growth. However, at least in this particular system, there seems to exist an affinity ceiling, beyond which a corresponding augmentation in the magnitude of the immune response could not be achieved. In addition, a significant decrease in the initial number of specific complexes, both of low and high affinity peptides, was observed in the first two hours post-loading. These findings underline an inherent limitation associated with the transient nature of MHC-binding by exogenous antigenic peptides, and reinforces the prospects of genetic modification of DCs.

A number of studies have indeed attempted to increase the actual frequency of the desired antigenic class I complexes on the cell surface, through genetic engineering of improved class I-peptide ligands. For example, one group (Mottez et al., 1995; Lone et al., 1998) has constructed a chimeric MHC class I molecule, in which the antigenic peptide was covalently linked to the amino terminal of the a chain. These proteins were expressed on the surface of transfected cells and were capable of eliciting a specific CTL response. However, using this approach, each antigenic peptide should be constructed with its own restricting a chain. To overcome this problem, another group (Uger and Barber, 1998) has attached the antigenic peptide to the amino terminal of the monomorphic β₂m. Primary T cells from mice, which had been immunized with the specific peptide, could indeed selectively lyse transfected cells, expressing these constructs. However, the cells used for expression in this study were deficient in MHC class I expression, due to a TAP transporter mutation. Yet, in spite of lack of competition from cytosol-derived peptides, level of peptide presentation was limited. Using a similar design, another study (Tafuro et al., 2001) has recently demonstrated reconstitution of MHC class I presentation in human cancer cells, but these, again, were class I-negative, due either to a TAP defect or to lack of β₂m expression. Although a non-mutated lymphoblastoid cell line was also included in this study and potentiated specific CTL lysis, there is no evidence as to the actual level of peptide presentation in these cells.

The construction of single-chain trimers (SCT) expressed as antigenic peptide-spacer-β₂m-spacer-heavy chain polypeptides (Yu et al., 2002; Lybarger et al., 2003; Huang et al., 2005) took this approach one step further. This design both favors the assembly of heterotrimers comprising the linked peptide and prevents their irreversible disengagement at the cell surface, since all covalently bound components are anchored to the plasma membrane and remain available for rebinding. Indeed, the resulting topology renders the binding groove 1000-fold less accessible to soluble peptide (Yu et al., 2002). A DNA vaccine encoding human papilloma virus (HPV) 16 E6 antigen protected 100% of immunized mice from an otherwise lethal challenge with an E6 expressing tumor (Huang et al., 2005).

Expression of TAAs by gene-modified DCs allows high level and prolonged peptide presentation, which can be significantly improved by rational targeting of the protein antigens to selected cellular compartments along the MHC-I presentation pathway. Viral vectors enable efficient DC transduction, drive robust protein expression and provide DC maturation stimuli. However, viruses still raise serious safety concerns, and concomitant anti-vector immunity often masks desired response and limits repeated administration. In contrast, DNA-, and especially mRNA-based vaccines are safer modalities, which confine expression only to proteins of interest. Contribution of cross-presentation to CTL priming following DNA vaccination has been demonstrated in animal models. Ex-vivo genetic manipulation of DCs by mRNA offers high transfection efficacy and unmatched safety and is becoming an attractive genetic modality for cancer therapy.

Autoimmune disorders are characterized by reactivity of the immune system to an endogenous antigen, with consequent injury to tissues. More than 80 chronic autoimmune diseases have been characterized that affect virtually almost every organ system in the body. The most common autoimmune diseases are insulin-dependent diabetes mellitus (IDDM), multiple sclerosis (MS), systemic lupus erythematosus (SLE), rheumatoid arthritis, several forms of anemia (pernicious, aplastic, hemolytic), thyroiditis, and uveitis.

Autoimmune diseases result from sustained adaptive immune responses mounted against innocuous self-antigens. The effector mechanisms that eventually cause tissue damage and disease are most likely those that take part in normal adaptive responses, and include production of specific antibodies, generation of immune complexes, inflammatory and cytotoxic T cells and activated macrophages. Regulatory T cells (Tregs) seem to play a crucial role in the development of autoimmune disorders (for review, see Tang & Bluestone, 200; Liu & Leung, 2006; Zwar et al., 2006). Therefore, suppressing Treg reactivity could play a crucial role in the development of autoimmune diseases.

A limited number of peptides derived from proteins involved in autoimmune diseases are associated with the onset of the disease. The immune responses to self-antigens are maintained by the persistent activation of self-reactive T cells. Removal of T cell populations that are associated with the autoimmune response should lead to prevention and/or cure of the disease. This model was demonstrated in NOD mice, where the removal of T-cell populations that recognize proinsulin II, prevented the onset of IDDM (French, et al., 1997).

Allograft rejection typically results from an overwhelming adaptive immune response against foreign organ or tissue. It is the major risk factor in organ transplantation and is the cause of post-transplantation complications. A major complication associated with bone marrow (BM) transplantation, known as graft-versus-host (GVH) reaction or graft-versus-host disease (GVHD), occurs in at least half of patients when grafted donor lymphocytes, injected into an allogeneic recipient whose immune system is compromised, begin to attack the host tissue, and the host's compromised state prevents an immune response against the graft. Alloreactivity is complex and involves many cell types as well as inflammatory factors. It is largely mediated by both CD8⁺ (CTL) and CD4⁺ (T_{H}) T cells (for review, see Douillard et al., 1999; Hernandez-Fuentes et al., 1999; Pattison and Krensky, 1997).

Allograft rejection results from proper recognition of foreign MHC and activation of the adaptive immune system and is carried out by direct or indirect pathways. The direct pathway, where T-cell receptors directly recognize intact allo-MHC with or without bound peptides on the surface of target cells, apparently accounts for most of the CTL function. The indirect pathway, where T-cell receptors recognize MHC allopeptides after processing and presentation, leads to the activation of T helper cells. These cells provide the necessary signals for the growth and maturation of effector CTLs and B cells leading to rejection (Sherman and Chattopadhyay, 1993; Watschinger, 1995).

The actual role of specific peptides in direct allorecognition is ambiguous. Some studies demonstrate that allorecognition is peptide-independent (Mullbacher et al., 1999; Smith et al., 1997), while others imply that specific peptides do contribute to allorecognition (Wang, et al., 1998). Allorecognition may, therefore, comprise peptide-independent, peptide-dependent or peptide-specific interactions. Tregs seem to play a major role in the induction and maintenance of tolerance to alloantigens, which bears important implications to the engraftment of transplants or the prevention of GVHD (for review, see Walsh et al., 2004).

Citation or identification of any reference in any section of this application shall not be construed as an admission that such reference is available as prior art to the present invention.

### SUMMARY OF THE INVENTION

The present invention relates, in one aspect, to a polynucleotide comprising a sequence encoding a polypeptide that is capable of high level presentation of antigenic peptides on antigen-presenting cells, wherein the polypeptide comprises a β₂-microglobulin molecule that is linked through its carboxyl terminal to a polypeptide stretch that allows the anchorage of the β₂-microglobulin molecule to the cell membrane, and through its amino terminal to at least one antigenic peptide comprising a MHC class I epitope, wherein said antigenic peptide is not related to an autoimmune disease.

In one embodiment, an epitope, which is an antigenic determinant of one sole antigen, is linked to the amino terminal of the β₂-microglobulin. In another embodiment, there are two or more epitopes that may be antigenic determinants of the same or of two or more different antigens. The epitopes/antigenic peptides may be derived from a tumor-associated antigen (TAA), from an infectious agent, e.g. a bacterial or viral protein, or they are TCR idiotypic peptides expressed by autoreactive T cells and BCR or antibody idiotypic peptides expressed by autoreactive B cells. These chimeric polypeptides are referred to herein as “double-chimeric β₂-microglobulin” (dcβ₂m).

In another aspect, the invention relates to a polynucleotide comprising a sequence encoding a polypeptide that is capable of high level presentation of antigenic peptides on antigen-presenting cells, wherein the polypeptide comprises a β2-microglobulin molecule that is linked through its carboxyl terminal to a polypeptide stretch that allows the anchorage of the β2-microglobulin molecule to the cell membrane, and through its amino terminal to at least one antigenic peptide comprising an MHC class I epitope, and said polypeptide stretch consists of a bridge peptide that spans the whole distance to the cell membrane, said bridge peptide being linked to the full or partial transmembrane and/or cytoplasmic domains of a molecule selected from the group consisting of a toll-like receptor (TLR) polypeptide, a CD40 polypeptide, and a TLR polypeptide and a CD40 polypeptide fused in tandem. The antigenic peptide in this case may be related to an autoimmune disease or to other diseases and disorders such as cancer and infectious diseases.

As used herein, the term “full or partial transmembrane and/or cytoplasmic domains of a TLR polypeptide and a CD40 polypeptide fused in tandem” means that the two domains are linked in tandem in a way that the biological functions of both the TLR and the CD40 peptides are preserved. A peptide-less β₂m linked to such elements can be used in the case of graft-versus-host disease, or transplant rejection.

In another aspect, the invention relates to a polynucleotide comprising a sequence encoding a polypeptide that is capable of high level presentation of antigenic peptides on antigen-presenting cells, wherein the polypeptide comprises a 2-microglobulin molecule that is linked through its carboxyl terminal to a polypeptide stretch that allows the anchorage of the β2-microglobulin molecule to the cell membrane, and said polypeptide stretch consists of a bridge peptide that spans the whole distance to the cell membrane, said bridge peptide being linked to the full or partial transmembrane and/or cytoplasmic domains of a molecule selected from the group consisting of a toll-like receptor (TLR) polypeptide, a CD40 polypeptide, and a TLR polypeptide and a CD40 polypeptide fused in tandem. These constructs can be used for treatment of autoimmune diseases by administering to an individual suffering from an autoimmune disease autologous T cells that have been transfected with such a construct and incubated with one or more antigenic peptides related to said autoimmune disease. These constructs can also be used for prevention and/or treatment of GVHD by administering to an individual undergoing transplantation autologous T cells that have been transfected with such a construct. These constructs can further be used for prevention and/or treatment of host-versus-graft rejection by administering to an individual undergoing transplantation donor. T cells that have been transfected with such a construct.

In another aspect, the present invention relates to a vector comprising a DNA molecule of the invention.

In a further aspect, the present invention relates to antigen-presenting cells (APCs), which express a dcβ₂m encoded by the DNA molecule of the invention as defined above. Any suitable professional APC can be used according to the invention such as dendritic cells, macrophages and B cells. In a preferred embodiment, the APC is a dendritic cell. Transfection or transduction of the cells is carried out by standard methods of recombinant DNA technology as well known to a person skilled in the art.

In one preferred embodiment, the APCs are capable of expressing a dcβ₂m polypeptide comprising at least one TAA peptide such as to present the TAA peptide(s) at a sufficiently high density to allow potent activation of peptide-specific cytotoxic T lymphocytes (CTL) capable of recognizing and binding to harmful tumor cells and causing their elimination or inactivation.

The present invention further provides a cancer vaccine comprising an agent selected from: (i) a DNA molecule encoding a dcβ₂m as defined herein wherein the at least one epitope linked to the amino terminal of β₂m is derived from at least one TAA; (ii) an expression vector comprising such DNA molecule (i); (iii) antigen presenting cells expressing a dcβ₂m as defined herein wherein the at least one epitope linked to the amino terminal of β₂m is derived from at least one TAA; (iv) antigen presenting cells (APCs) expressing a single-chimeric β₂-microglobulin (scβ₂m) molecule as defined herein consisting of a β₂-microglobulin molecule linked through its carboxyl terminal to a polypeptide stretch that allows the anchorage of the β₂-microglobulin molecule to the cell membrane; and (v) APCs as defined in (iv) that have been pulsed with at least one TAA peptide.

The present invention still further provides pharmaceutical compositions for use in inducing a class I-restricted CTL response in a mammal comprising cells expressing a dcβ₂m of the invention.

The present invention further provides a method of immunizing a mammal against a tumor-associated antigen comprising the step of immunizing the mammal with a cellular vaccine, which comprises an antigen presenting cell transfected with a polynucleotide comprising a sequence encoding a scβm₂, wherein said cells have been pulsed with at least one antigenic peptide derived from at least one tumor-associated antigen.

### BRIEF DESCRIPTION OF THE FIGURES

FIGS. 1A-1B depict the construction and expression of dcβ₂m. FIG. 1A is a sketch of the dcβ₂m polypeptide associated on the cell surface with a MHC class I heavy (α) chain, while the antigenic peptide is in the binding groove. The transmembrane and cytoplasmic domains are derived from either the mouse CD3 ζ chain or the MHC class I heavy chain H-2K^{b}, and are covalently attached, via a short bridge, to the carboxyl terminus of human or mouse β₂m. The antigenic peptide is attached to the amino terminal of β₂m via a short linker with the sequence G₄S(G₃S)₂. FIG. 1B is a scheme of the genetic construct: pr, promoter; lead, leader peptide; p, antigenic peptide; li, linker peptide; br, bridge. Important restriction sites are indicated.

FIGS. 2A-2C show flow cytometry analysis of MD45 parental cells (FIG. 2A) and transfectants 427-44 (Ha) cells (expressing the Ha₂₅₅₋₂₆₂ dcβ₂m) (FIG. 2B) and 425-44 (NP) cells (expressing NP₅₀₋₅₇ dcβ2m) (FIG. 2C). Cells were analyzed with primary antibodies against H-2K^{k} (clone AF3-12.1), hβ₂m (clone BM-63) and K^{k}/Ha₂₅₅₋₂₆₂ complex (Fab13.4.1) and detected with secondary goat anti-mouse IgG (Fab-specific)-FITC conjugated polyclonal antibodies.

FIG. 3 shows stimulation of the MD45 transfectants 425-44, 427-24 and 892S-36 (see Table 1 hereinafter) by different MHC-I allele-specific antibodies. Indicated cells at 5×10⁵/ml in 100 μl were incubated in wells of a microtiter plate pre-coated with the different antibodies at 5 μg/ml and then subjected to an in-cell X-Gal staining. Anti-K^{k} is AF3-12.1 and anti-K^{d} is SF1-1.1. Anti-TCR is the hamster anti-mouse CD3ε mAb 2C11, which served as a positive control for activation.

FIGS. 4A-4C show FACS analysis of RMA (FIG. 4A), RMA-S (FIG. 4B) and transfectant Y317-2 (expressing OVA₂₅₇₋₂₆₄ linked to human membranal β₂m) cells (FIG. 4C). Antibodies were: anti-H-2 D^{b} (28-14-8); anti-H-2K^{b} (20-8-4); anti-hβ₂m (BM-63) and anti-K^{b}-OVA₂₅₇₋₂₆₄ (25-D1.16). Cells were grown for 24 hours in serum-free medium prior to staining at both 27° C. and 37° C.

FIGS. 5A-5G show that a K^{b}/OVA₂₅₇₋₂₆₄-specific T cell hybridoma is activated by cells expressing OVA₂₅₇₋₂₆₄ dcβ₂m. B3Z cells, an H-2K^{b}-restricted OVA₂₅₇₋₂₆₄-specific T cell hybridoma, were incubated with: FIG. 5A, no stimulation; FIG. 5B, Plastic-bound (5 μg/ml) anti-CD34 mAb (2C1); FIG. 5C, RMA cells; FIG. 5D, RMA cells loaded with synthetic OVA₂₅₇₋₂₆₄ at 2 μg/ml as a positive control; FIG. 5E, Y314-7 cells; 5F. Y317-2 cells; 5G. Y318-7 cells as a negative control. All cells were at 5×10⁵/ml. Cells were stained with X-Gal and visualized under a microscope.

FIGS. 6A-6B depict construction and expression of scβ₂m. FIG. 6A is a sketch of the scβ₂m polypeptide. The transmembrane and cytoplasmic domains are derived from either the mouse CD3 ζ chain or the MHC class I heavy chain H-2K^{b}, and are covalently attached, via a short bridge, to the carboxyl terminus of human or mouse β₂m. FIG. 6B is a scheme of the genetic construct: pr, promoter; lead, leader peptide; p, antigenic peptide; li, linker peptide; br, bridge. Important restriction sites are indicated.

FIG. 7 shows stabilization of MHC class I molecules by membranal β₂m. KD21-4 and KD21-6 RMA-S transfectants and parental RMA-S and RMA cells were grown in serum-free medium for 24 hours at 27° C. and 37° C. and then stained with anti-H-2 Db (28-14-8) and anti-hβ₂m (BM-63) mAbs. FACS analysis was performed with FACSCalibur (BD Biosciences).

FIG. 8 is a graph showing the ability of KD21-6 and D323-4 transfectants to bind exogenously added synthetic OVA₂₅₇₋₂₆₄ peptide through H-2K^{b}, in comparison with parental RMA-S cells. The cells were grown at 37° C. for 24 hours in serum-free medium and were then incubated for 42 hours with serial dilutions of synthetic OVA₂₅₇₋₂₆₄. Cells were stained with mAb 25.D1-16 and FACS analysis was performed with FACSCalibur. Mean fluorescence intensity was calculated using CellQuest software.

FIG. 9 is a graph showing generation of antigen specific CTLs following cell immunization. RMA-S and RMA-S/OVA (negative controls), RMA-S loaded with OVA₂₅₇₋₂₆₄ and RMA/OVA (positive controls), and transfectants Y317-2 and Y314-7 were injected i.p. twice at 10-day interval. Ten days after the second immunization, CTLs were prepared and the indicated cells (Y317-2, Y314-7 and RMA-S) were used as target cells in a cell cytotoxicity assay at effector/target ratio of 50:1. Histogram shows percent specific lysis.

FIG. 10 shows FACS analysis of RMA-S, KD21-6 and Y340-13 cells. Cells were analyzed with a primary antibody against hβ₂m (clone BM-63) and detected with secondary goat anti-mouse IgG (Fab-specific)-FITC conjugated polyclonal antibodies.

FIGS. 11A-11B show inhibition of tumor growth. MO5 tumor cells (1×10⁵/mouse) were injected s.c. to female B6 mice (8-12 week old). Eight days later, when tumor diameter reached 3-4 mm, mice were divided to groups of ten and were immunized i.p. 4 times at 7 day intervals (days 8, 15, 22, 29) with irradiated Y317-2(hOVA) or RMA-S cells loaded with 50 μg/ml OVA₂₅₇₋₂₆₄ or with PBS only (non-immunized). FIG. 11A. Tumor progression. Local tumor dimensions were measured with calipers. The average of tumor diameters (in millimeters) in the course of 50 days is presented. FIG. 11B. Survival of immunized mice. Mice from the same experiment were monitored daily and were sacrificed when moribund, which corresponded to tumor diameter of approximately 20 mm. Fraction of surviving mice in each group is presented. Data are representative of two independent experiments with similar results. The results are presented as mean+SEM. Both panels present p values calculated for the two groups of immunized mice.

FIG. 12 depicts the construction and expression of the dcβ₂m of FIG. 1A in which the transmembrane (tm) and cytoplasmic (cyt) domains are derived from either a TLR or CD40, and are covalently attached, via a short bridge, to the carboxyl terminus of human or mouse β₂m.

FIGS. 13A-13D depict flow cytometry analysis of stable transfectants of the mouse macrophage RAW264.7 cell line. FIGS. 13A-13B: GA467-8 and 11, respectively, with mTLR4; FIG. 13C: GA323 with the 323 construct; and FIG. 13D: GA518-18 with mTLR2. Staining was performed with a mouse anti-hβ₂m mAb and FITC-conjugated goat anti-mouse IgG Abs. Filled histograms, RAW264.7 background.

FIGS. 14A-14B depict a constitutively activated phenotype conferred on transfected APC (human monocytic THP-1) cell line, which express peptide-less β₂m harboring the TLR4 tm and cyt portion, as analyzed by semi-quantitative reverse-transcriptase-(RT)-PCR. FIG. 14A presents an analysis of human monocytic THP-1cells for expression of IL-1β, IL-6 and IL-12. RT-PCR was performed on mRNA prepared from non-treated positive (1499-3) and negative (1499-4) THP-1 transfectants and parental cells, and parental cells treated for 24 hours with LPS (0.5 μg/ml) as a positive control. PCR amplification was recorded after 20-35 cycles in 5 cycle intervals (shown are results obtained after 30 cycles). Expression of the indicated cytokine genes was normalized according to GAPDH expression, using PhosphorImager analysis. FIG. 14B shows analysis of mouse RAW264.7 macrophages for expression of IL-1β. A transfectant expressing hβ₂m-H-2K^{b} construct (left) served as a negative control. Shown is an ethidium-bromide stained agarose gel with RT-PCR samples generated from 1 and 0.1 μg RNA from negative control, LPS-treated (5 μg/ml, 2 h) parental RAW264.7 cells and two hβ₂m-TLR4-transfected clones.

FIGS. 15A-15B depict a constitutively activated phenotype conferred on transfected APC lines, which express dcβ₂m harboring the TLR4 tm and cyt portion and an antigenic peptide. FIG. 15A shows a flow cytometry analysis of the parental RAW264.7 cells (left panel) and the transfectants Ey568-39 (middle panel) and Ey569-31 (right panel) for expression of surface hβ₂m by stable transfectants. FIG. 15B presents an ethidium-bromide stained agarose gel with RT-PCR samples generated from 1 μg of RNA from negative controls (RAW264.7 and clone Ey568-39) and LPS-treated (5 μg/ml, 2 h) parental RAW264.7 cells and clone Ey569-31), using PCR primers specific for TNFα and GAPDH (for data normalization).

FIGS. 16A-16B depict the ability of an antigenic peptide linked to a TLR4- and TLR2-bearing dcβ₂m construct to stimulate the mouse T cell hybridoma CHIB2. Immature mouse DC XS52 cells (FIG. 16A) and RAW 264.7 macrophages (H-2^{d}) (FIG. 16B) were transfected with 5 μg in-vitro transcribed mRNA encoding the mouse insulin B-chain heteroclitic peptide G9V linked to hβ₂m-mTLR4, hβ₂m-mTLR2 or hβ₂m-H-2K^{b} or irrelevant RNA as control for transfection efficiency. After 48 hours, transfected cells were co-incubated with CHIB2 cells at 1:1 ratio for 24 hours and cells were then subjected to a LacZ enzymatic assay to assess T cell activation, using the colorimetric substrate chlorophenol red-β-D-galactopyranoside (CPRG). Results are shown as OD₅₇₀ with OD₆₃₀ as reference.

FIGS. 17A-17B depict a flow cytometry analysis for the influence of dcβ₂m expression on the maturation program of human DCs cultured ex-vivo. Immature human DCs were transfected with 5 μg of in-vitro transcribed RNA encoding either gp100₂₀₉₋₂₁₇-hβ₂m-TLR4 (designated ‘501’) or gp100₂₀₉₋₂₁₇-hβ₂m-A2 (‘541’). Alternatively, these cells were treated with 5 μg/ml LPS, or allowed to mature in the presence of the maturation cytokine cocktail. Twenty four hours post-transfection cells were subjected to flow cytometry analysis for expression of CD86 (B7.2) as a surface marker indicative of DC maturation. Mean fluorescence intensity (MFI) values, as calculated by the CellQuest software, are shown for each treatment. MDC, mature DCs; IMDC, immature DCs.

FIGS. 18A-18B depict a flow cytometry analysis for expression of hβ₂m-CD40 in A20 (a mouse B cell lymphoma expressing CD40) transfectants RB340-1-21 and RB340-2-3, respectively. Staining was performed with a mouse anti-hβ₂m mAb and FITC-conjugated goat anti-mouse IgG antibodies. N.C and P.C. denote negative (2^{nd} Ab only) and hβ₂m positive control, respectively.

FIGS. 19A-19B show function of the CD40 monomeric activation domain in the A20 transfectant RB340-1-10. Cells were incubated for 1 hour with the indicated antibodies (Abs): hamster anti-mouse CD40, mouse anti-hβ₂m, and mouse anti-H-2K^{d}, and then harvested. A calibrated amount of detergent lysates were subjected to PAGE and subsequent immunoblot analysis, first with an anti-Iκ3αmAb and then, following stringent stripping of bound Abs, with an anti-mouse tubulin mAb. 19A. Protein gel. 19B. Relative level of IκBα. Signal intensity was determined and normalized using the TINA software. The amount of IκBα with no stimulating Ab was considered 100% and all other values were calculated and plotted accordingly.

### DETAILED DESCRIPTION OF THE INVENTION

Duration of the functional MHC classI/peptide complex on the cell surface is governed by the affinity of the peptide for the MHC molecule. Dissociation of the peptide from its binding groove in the α heavy chain, results in practically irreversible disruption of the ternary complex formed between the α chain, β₂m and peptide. Both latter components are not anchored to the cell membrane and immediately detach from the cell, while the α chain is later internalized. Stabilization of a particular class I/peptide complex by enabling fast re-association is therefore likely to result in high level of presentation of the antigenic peptide.

In one aspect, the concept underlying the present invention is that connecting at least one epitope to one end (the amino terminal) of β₂m and anchoring this polypeptide to the cell membrane through its other end (the carboxyl terminal), will provide an exceedingly high level of the antigenic peptide directly to the ER in a TAP- and proteasome-independent manner and substantially increase complex stability, and consequently, the level of presentation of this peptide.

WO 01/91698 of the same applicants, hereby incorporated by reference in its entirety as if fully disclosed herein, discloses the development by genetic engineering of a novel MHC class I configuration, in which the β₂m light chain is anchored to the cell membrane, while harboring an antigenic peptide related to an autoimmune disease fused to its amino terminal. Expression of this construct results in an exceptionally high level of the MHC-peptide complex on the surface of transfected cells, despite competition from normally presented peptides. Thus, an influenza virus hemagglutinin-derived peptide (Ha₂₅₅₋₂₆₂), restricted by the mouse class I allele K, was linked to the amino terminal of β₂m by genetic engineering, while the carboxyl terminal was anchored to the membrane of transfected, K^{k}-expressing cells. Analyses performed with an anti-K^{k} mAb and another mAb, which shows exquisite specificity to the K^{k}/Ha₂₅₅₋₂₆₂ complex, revealed high levels of the complex on the surface of transfected cells. It should be emphasized that efficient pairing of Ha₂₅₅₋₂₆₂ with K^{k} through this double-chimeric β₂m (dcβ₂m) was achieved in-spite of strong competition from cytosolic-derived K^{k}-restricted peptides. Although data cannot be directly compared, it is to be noted that high level of surface class I-bound peptide by expression of non-membrane-attached β₂m/peptide alone, could not be directly demonstrated in previous studies (Uger and Barber, 1998; Tafuro et al., 2001). Membranal anchorage of dcβ₂m is therefore likely to result in substantial augmentation in the overall density of desired class I antigenic peptides on the cell surface, thus offering a novel and unique tool for CTL induction.

The main objectives of the present invention are to develop both a cell based-vaccine and a DNA vaccine, based on membranal β₂m carrying at least one antigenic peptide covalently bound to its amino terminal. In one embodiment, the antigenic peptide is not a peptide related to an autoimmune disease. In another embodiment, the antigenic peptide is a peptide related to an autoimmune disease.

As used herein, the terms “antigenic peptide” or “peptide or epitope derived from an antigen” mean both a peptide having a sequence comprised within the sequence of said antigen or an altered sequence, in which one or more amino acid residues have been replaced by different amino acid residues, which may bear higher affinity for the MHC class I molecule.

Thus, in one aspect, the present invention provides a polynucleotide comprising a sequence encoding a polypeptide that is capable of high level presentation of antigenic peptides on antigen-presenting cells, wherein the polypeptide comprises a β₂-microglobulin molecule that is linked through its carboxyl terminal to a polypeptide stretch that allows the anchorage of the β′₂-microglobulin molecule to the cell membrane, and through its amino terminal to at least one antigenic peptide comprising a MHC class I epitope, wherein said antigenic peptide is not related to an autoimmune disease.

In one embodiment, the polypeptide stretch at the β₂-microglobulin carboxyl terminal consists of a bridge peptide, which spans the whole distance to the cell membrane, said bridge peptide being linked to a sequence which can exert the required anchoring function. The bridge peptide has preferably about 10-15 amino acid residues, and more preferably, has a sequence comprised within the membrane-proximal sequence of a class I heavy chain HLA molecule. In a most preferred embodiment, this bridge peptide has 13 amino acid residues comprised within the extracellular membrane-proximal sequence of the class I heavy chain HLA-A2 molecule, and is the peptide of SEQ ID NO: 1, of the sequence LRWEPSSQPTIPI.

In one embodiment, the anchoring sequence to which the bridge peptide is linked is the full or partial transmembrane and/or cytoplasmic domain of a molecule selected from the group consisting of: (i) a human MHC class I molecule consisting of an HLA-A, HLA-B or HLA-C molecule; (ii) a costimulatory B7.1, B7.2 or CD40 molecule; and (iii) a signal transduction element capable of activating T cells or antigen-presenting cells.

In one embodiment, the anchoring residue (i) above is a sequence consisting of the transmembrane and cytoplasmic domains from the MHC class I heavy chain HLA-A2 molecule, of the SEQ ID NO: 2, of the sequence:

VGIIAGLVLFGAVITGAVVAAVMWRRKSSDRKGGSYSQAASSDSAQ
GSDVSLTACKV

In another embodiment, the anchoring residue (iii) above is the intracellular region of a suitable signal transduction element capable of activating T cells such as, but not being limited to, a component of T-cell receptor CD3 such as the zeta (ζ) or eta (η) polypeptide, a B cell receptor polypeptide or an Fc receptor polypeptide, or it is a suitable signal transduction element capable of activating antigen-presenting cells, for example, but not being limited to, a toll-like receptor (TLR) polypeptide. The cytoplasmic regions of the CD3 chains contain a motif designated the immunoreceptor tyrosine-based activation motif (ITAM), which has been shown to associate with cytoplasmic tyrosine kinases and to participate in signal transduction following TCR-mediated triggering. This motif is found in a number of other receptors including the Ig-α/Ig-β heterodimer of the B-cell receptor complex and Fc receptors for IgE and IgG, and three copies of it are found in the long cytoplasmic domains of the ζ and η chains.

In a preferred embodiment, the anchoring residue of the chimeric molecule comprises the transmembranal and cytoplasmic regions of the human T-cell receptor CD3 ζ polypeptide, a signal transduction element capable of activating T cells.

In another embodiment, the signal transduction element capable of activating T cells comprises the transmembranal and cytoplasmic regions of a B-cell receptor polypeptide such as the Ig-α or Ig-β chain, the cytoplasmic tails in both being long enough to interact with intracellular signaling molecules. In a further embodiment, the signal transduction element comprises the transmembranal and cytoplasmic regions of Fc receptor polypeptides such as FcεRI, FcγRI or FcγRIII chains. FcεRI, a high-affinity receptor expressed on the surface of mast cells and basophils, contains four polypeptide chains: an α and a β chain and two identical disulfide-linked γ chains that extend a considerable distance into the cytoplasm and each has an ITAM motif. FcγRI, or CD64, is the high affinity receptor for IgG, expressed mainly on macrophages, neutrophils, eosinophils and dendritic cells. It comprises an α chain and two disulfide-linked γ chains. This structure is also typical to FcγRIII, or CD16, which is the low affinity receptor for IgG, found on NK cells, eosinophils, macrophages, neutrophils and mast cells. CD3 ζ chain is found instead of the γ chain in a fraction of FcγRIII.

In still a further embodiment, the anchoring residue to which the bridge peptide is linked through its carboxyl terminal is a glycosylphosphatidylinositol (GPI)-anchor sequence, preferably the GPI-anchor peptide of SEQ ID NO:3, of the sequence FTLTGLLGTLVTMGLLT (from the protein DAF-complement decay-accelerating factor precursor or CD55 antigen; SWISSProt ID P08174, positions 365-381).

In one embodiment, the polynucleotide of the invention comprises a sequence encoding a polypeptide as defined in which the at least one non-autoimmune disease related antigenic peptide comprising a MHC class I epitope is linked to the β₂-microglobulin amino terminal directly. In another embodiment, the at least one antigenic peptide is linked to the β₂-microglobulin amino terminal through a peptide linker.

In one embodiment, the at least one antigenic peptide is at least one antigenic determinant of one sole antigen.

In another embodiment, the at least one antigenic peptide is at least one antigenic determinant of each one of at least two different antigens.

In one preferred embodiment of the invention, the at least one non-autoimmune disease related antigenic peptide comprising a MHC class I epitope linked to the β₂-microglobulin amino terminal is derived from a tumor-associated antigen (TAA) such as, but not limited to, alpha-fetoprotein, BA-46/lactadherin, BAGE (B antigen), BCR-ABL fusion protein, beta-catenin, CASP-8 (caspase-8), CDK4 (cyclin-dependent kinase 4), CEA (carcinoembryonic antigen), CRIPTO-1 (teratocarcinoma-derived growth factor), elongation factor 2, ETV6-AML1 fusion protein, G250/MN/CAIX, GAGE, gp100 gp100 (glycoprotein 100)/Pmel17, HER-2/neu (human epidermal receptor-2/neurological), intestinal carboxyl esterase, KIAA0205, MAGE (melanoma antigen), MART-1/Melan-A (melanoma antigen recognized by T cells/melanoma antigen A), MUC-1 (mucin 1), N-ras, p53, PAP (prostate acid phosphatase), PSA (prostate specific antigen), PSMA (prostate specific membrane antigen), telomerase, TRP-1/gp75 (tyrosinase related protein 1, or gp75), TRP-2, tyrosinase, and uroplakin Ia, Ib, II and III.

Examples of TAA peptides include, without being limited to, the following antigenic peptides:
(i) the HLA-A2 restricted human alpha-fetoprotein peptide GVALQTMKQ (SEQ ID NO:4) associated with liver tumors;
(ii) the HLA-Cw16 restricted human BAGE-1 peptide AARAVFLAL (SEQ ID NO:5);
(iii) the HLA-A2 restricted human BCR-ABL fusion protein (b3a2) peptide SSKALQRPV (SEQ ID NO:6) associated with chronic myeloid leukemia;
(iv) the HLA-A24 restricted human beta-catenin peptide SYLDSGIHF (SEQ ID NO:7) associated with melanoma;
(v) the HLA-A2 restricted human CDK4 peptide ACDPHSGHFV (SEQ ID NO:8) associated with melanoma;
(vi) the HLA-A2 restricted human CEA peptide YLSGANLNL (SEQ ID NO: 9) associated with gut carcinoma;
(vii) the HLA-A68 restricted human elongation factor 2 peptide ETVSEQSNV (SEQ ID NO:10) associated with lung squamous cell carcinoma;
(viii) the HLA-A2 restricted human ETV6-AML1 fusion protein peptide RIAECILGM (SEQ ID NO:11) associated with acute lymphoblastic leukemia;
(ix) the HLA-A2 restricted human G250 peptide HLSTAFARV (SEQ ID NO: 12) associated with stomach, liver and pancreas tumors;
(x) the HLA-Cw6 restricted human GAGE-1,2,8 peptide YRPRPRRY (SEQ ID NO:13);
(xi) the gp100 human peptides associated with melanoma HLA-A2 restricted KTWGQYWQV (SEQ ID NO:14), (A)MLGTHTMEV (SEQ ID NO:15), ITDQVPFSV (SEQ ID NO: 16), YLEPGPVTA (SEQ ID NO: 17), LLDGTATLRL (SEQ ID NO: 18), VLYRYGSFSV (SEQ ID NO: 19), SLADTNSLAV (SEQ ID NO:20), RLMKQDFSV (SEQ ID NO:21), RLPRIFCSC (SEQ ID NO:22), and the HLA-A3 restricted LIYRRRLMK (SEQ ID NO:23), ALLAVGATK (SEQ ID NO:24), IALNFPGSQK (SEQ ID NO:25) and ALNFPGSQK (SEQ ID NO:26);
(xii) the HLA-A2 restricted human HER-2/neu ubiquitous peptide KIFGSLAFL (SEQ ID NO: 27);
(xiii) the HLA-B7 restricted human intestinal carboxyl esterase peptide SPRWWPTCL (SEQ ID NO:28) associated with liver, intestine and kidney tumors;
(xiv) the HLA-B44 restricted human KIAA0205 peptide AEPINIQTW (SEQ ID NO:29) associated with bladder tumor;
(xv) the MAGE-1 peptides HLA-A1 restricted human EADPTGHSY (SEQ ID NO:30) and HLA-A3 restricted human SLFRAVITK (SEQ ID NO:31);
(xvi) the MAGE-3 peptides HLA-A1 restricted human EVDPIGHLY (SEQ ID NO:32) and HLA-A2 restricted human FLWGPRALV (SEQ ID NO:33);
(xvii) the HLA-A2 restricted human MART-1/Melan-A peptide (E)AAGIGILTV (SEQ ID NO:34) associated with melanoma;
(xviii) the HLA-A2 restricted human MUC-1 peptide STAPPVHNV (SEQ ID NO:35) associated with glandular epithelia carcinoma;
(xix) the HLA-A1 restricted human N-ras peptide ILDTAGREEY (SEQ ID NO:36) associated with melanoma;
(xx) the HLA-A2 restricted human p53 ubiquitous peptide LLGRNSFEV (SEQ ID NO:37);
(xxi) the HLA-A2 restricted human PSA peptides FLTPKKLQCV (SEQ ID NO:38) and VISNDVCAQV (SEQ ID NO:39) associated with prostate carcinoma;
(xxii) the HLA-A2 restricted human telomerase peptide ILAKFLHWL (SEQ ID NO: 40) associated with testis, thymus, bone marrow, and lymph nodes carcinomas;
(xxiii) the HLA-A31 restricted human TRP-1 peptide MSLQRQFLR (SEQ ID NO:41) associated with melanoma;
(xxiv) the HLA-A2 restricted human TRP-2 peptides LLGPGRPYR (SEQ ID NO:42), SVYDFFVWL (SEQ ID NO:43), and TLDSQVMSL (SEQ ID NO:44) associated with melanoma;
(xxv) the HLA-A68 restricted human TRP2-INT2 peptide EVISCKLIKR (SEQ ID NO:45); and
(xxvi) the HLA-A1 restricted human tyrosinase peptide KCDICTDEY (SEQ ID NO:46) associated with melanoma.

This list is presented only as examples of TAA peptides that can be used according to the invention. However, it is intended to encompass within the scope any TAA peptide known or to be discovered in the future as periodically published in Cancer Immunity, a Journal of the Academy of Cancer Immunology, at the website http://www.cancerimmunity.org/peptidedatabase/Tcellepitopes.htm.

In one embodiment of the invention, the polynucleotide encodes a polypeptide comprising at least one antigenic determinant of one sole TAA. In another preferred embodiment, the polynucleotide encodes a polypeptide comprising at least one antigenic determinant of each one of at least two different TAAs.

Thus, in some applications according to the invention, it may be desired to link more than one epitope to the amino terminal of the anchored β₂m. In this way, the product of a single DNA molecule can mediate the induction of CTL clones directed at different epitopes from the same TAA, or from two or more different TAAs, restricted by one or more HLA class I allelic products.

In one embodiment, the two or more epitopes may be derived from the same antigen. For example, at least 9 different HLA-A2 binding peptides and 4 different HLA-A3 binding peptides derived from the melanoma-associated antigen gp100 have been identified. A melanoma patient, who carries both HLA-A2 and HLA-A3, can, in principle, mount CTL responses to these 13 different gp100-derived peptides.

Thus, in one preferred embodiment, the at least one antigenic peptide is at least one HLA-A2 binding peptide and at least one HLA-A3 binding peptide derived from the melanoma-associated antigen gp100, more preferably at least one gp100 HLA-A2 binding peptide selected from the group consisting of SEQ ID NO: 14, 15, 16, 17, 18, 19, 20, 21 and 22, and at least one gp100 HLA-A3 binding peptide selected from the group consisting of SEQ ID NO: 23, 24, 25 and 26.

In another embodiment, this strategy can be employed to elicit a CTL response to more than one antigenic molecule by using a single gene encoding epitopes of two different TAAs. For example, the same sequence can harbor peptides from gp100 and Melan-A/MART-1, both associated with melanoma, and harbor several HLA-A2-binding peptides, preferably at least one gp100 HLA-A2 binding peptide selected from the group consisting of SEQ ID NO: 14, 15, 16, 17, 18, 19, 20, 21 and 22, and at least one Melan-A/MART-1 HLA-A2 binding peptide selected from the group consisting of SEQ ID NO: 34. Similarly, peptides from different antigens, which bind different class I alleles can be incorporated on the same construct, e.g., HLA-A3-restricted gp100 and HLA-A2-restricted Melan-A/MART-1 peptide(s). Similarly, other combinations of different TAAs related to melanoma can be formed using one or more of the melanoma-associated TAAs described above, e.g. peptides derived from beta-catenin, CDK4, gp100, Melan-A/MART-1, N-ras, TRP-1, TRP-2, and tyrosinase.

In another preferred embodiment of the invention, the at least one non-autoimmune disease related antigenic peptide comprising a MHC class I epitope linked to the β₂-microglobulin amino terminal is derived from an antigen from a pathogen selected from the group consisting of a bacterial, a viral, a fungal and a parasite antigen.

Examples of antigens derived from pathogenic, e.g. infectious, agents are, without being limited to, antigens derived from an organism selected from the group comprising: human immunodeficiency virus HIV (Takahashi et al., 1993), varicella zoster virus, herpes simplex virus type 1 (HSV-1), herpes simplex virus type 2 (HSV-2), human cytomegalovirus (CMV), dengue virus, hepatitis A, B, C or E, respiratory syncytial virus, human papilloma virus, influenza virus, Hib, meningitis virus, Salmonella, Neisseria, Borrelia, Chlamydia, Bordetella, Streptococcus, Mycoplasma, Mycobacteria, Haemophilus, Plasmodium or Toxoplasma, stanworth decapeptide; and TCR idiotypic peptides shared by autoreactive T cells (Cohen and Weiner, 1998; Offner et al., 1999; Kumar and Sercarz, 2001).

In one preferred embodiment, the pathogen antigen is a viral antigen such as, but not limited to, hepatitis virus, cytomegalovirus, or HIV viral antigen consisting of an HIV protein selected from the group consisting of the HIV-1 regulatory proteins Tat and Rev and the HIV envelope protein, in which case the antigenic peptide derived therefrom has the sequence RGPGRAFVTI (SEQ ID NO: 47).

In one embodiment of the invention, the polynucleotide encodes a polypeptide comprising at least one antigenic determinant of one sole pathogen antigen. In another preferred embodiment, the polynucleotide encodes a polypeptide comprising at least one antigenic determinant of each one of at least two different pathogen antigens. In this way, the product of a single DNA molecule can mediate the induction of CTL clones directed at different epitopes from the same viral antigen or from two or more different viral antigens, restricted by one or more HLA class I allelic products. For example, against AIDS, a combination of epitopes derived from each of the Tat, Rev and the HIV envelope proteins, may be used.

In yet a further embodiment of the invention, the at least one non-autoimmune disease related antigenic peptide comprising a MHC class I epitope linked to the β₂-microglobulin amino terminal is at least one idiotypic peptide expressed by autoreactive T lymphocytes. The idiotypic peptide is preferably derived from a CDR (complementarity-determining region), more preferably CDR3, of an immunoglobulin or of a TCR chain, and it may also contain CDR flanking segments.

This embodiment is suitable for some applications according to the invention that may require the covalent linking of longer polypeptide stretches, which may contain one or more epitopes of unknown class I binding properties. For example, idiotypic peptides derived from CDRs (especially CDR3) of immunoglobulin or TCR polypeptide chains can be employed for the induction of CTL response against lymphomas and leukemias of both B cell and T cell origin (Wen and Lim, 1993; Berger et al., 1998) or against autoreactive T cell clones (Kumar et al., 1995). However, many of these sequences are clonotypic in nature and there are no preliminary data concerning class I binding capacity of peptides they comprise. In such cases, longer DNA inserts, encoding, for example, not only the relevant CDR3 sequence, but also parts of its flanking FR3 and FR4 segments can be cloned directly from tumor cells or autoreactive T cell clones associated with an autoimmune disease. If the encoded stretch contains one or more peptides which can bind one or more of the patient's HLA class I products, the obtained dcβ₂m will induce CTLs of the corresponding specificities.

This task can be accomplished by the genetic insertion of the fragment encoding the longer peptide into the expression vector between the sequence encoding the leader peptide (the leader peptide or signal peptide is the peptide stretch at the amino terminal of any newly synthesized polypeptide chain, which is to be translocated to the ER) and the sequence coding for the linker peptide. The fragment encoding the longer peptide can be prepared with the use of synthetic oligonucleotides or as a PCR product (as for the CDR3 idiotypic peptides, using sets of FR3- and FR4-specific primers), or by any other procedure commonly used for molecular cloning. This design is based on the observations that MHC class I molecules can accommodate longer peptides than the canonical size of 8-10 amino acids. This most likely occurs by protrusion rather than by bulging (Stryhn et al., 2000) and shows preference to carboxyl terminal rather than to amino terminal extensions (Horig et al., 1999). It is predicted that in each assembly event in the ER of a relevant MHC class I molecule, a different peptide from the same dcβ₂m gene product can associate with the nascent MHC class I heavy chain. Following this association, the amino terminal protrusion can be trimmed by an ER aminopeptidase, operative in the early secretory pathway, as suggested by Snyder et al., 1994, and recently identified as the ER aminopeptidase ERAAP (Serwold et al., 2002) or ERAP1 (York et al., 2002; Saric et al., 2002), which trims precursors to MHC class I-presented peptides. The mature class I molecule will then be ready for transportation to the cell membrane. The rest of the long peptide may still link through its carboxyl terminal to the membranal β2m. Hence, enhanced complex stability and, concomitantly, high level of presentation are expected. In this manner, a panel of ligands can be formed in the APCs for induction of CTLs with different specificities, as the result of delivery of a single gene. This prediction also pertains to idiotypic peptides: an epitope can be embedded anywhere along the cloned sequence, and, similarly, the amino terminal protrusion will be cleaved. It is highly likely that there will be a functional limitation to the size of the linked stretch, and that secondary structures formed within this stretch will interfere with the ability of at least some of the embedded epitopes to be properly presented.

In a more preferred embodiment of the invention, the polynucleotide of the invention as described hereinbefore is an expression vector and comprises a vector and regulatory sequences along with the polynucleotide sequence.

In another aspect, the present invention provides an expression vector comprising a polynucleotide of the invention as described hereinbefore.

Any suitable mammalian expression vector can be used such as, but not limited to, the pCI mammalian expression vectors (Promega, Madison, Wis., USA), pCDNA3 expression vectors (Invitrogen, San Diego, Calif.) and pBJ1-Neo. The expression vector may also be a plasmid DNA in which the polynucleotide sequence is controlled by a virus, e.g. cytomegalovirus, promoter, or, most preferably, the expression vector is a recombinant viral vector such as, but not limited to, pox virus or adenovirus or adeno-associated viral vector.

In a further aspect, the present invention provides an antigen-presenting cell (APC) transfected with a polynucleotide comprising a sequence encoding a dcβ₂m of the invention, i.e. a polypeptide comprising a β₂-microglobulin molecule that is linked through its carboxyl terminal to a polypeptide stretch that allows the anchorage of the β₂-microglobulin molecule to the cell membrane, and through its amino terminal to at least one antigenic peptide comprising a MHC class I epitope.

The APC may be a macrophage, a B cell, a fibroblast and, more preferably, a dendritic cell. In a preferred embodiment, the antigenic peptide for use in the embodiments described above is a peptide not related to an autoimmune disease.

In one embodiment, the at least one antigenic peptide in the antigen-presenting cell is at least one peptide derived from at least one TAA. Said cell is capable of presenting the at least one TAA peptide at a sufficiently high density to allow potent activation of peptide-specific cytotoxic T lymphocytes (CTL) capable of recognizing and binding to harmful tumor cells and causing their elimination or inactivation.

In another embodiment, the at least one antigenic peptide in the antigen-presenting cell is at least one peptide derived from an antigen from a pathogen selected from the group consisting of a bacterial, a viral, a fungal and a parasite antigen.

In another embodiment, the at least one antigenic peptide in the antigen-presenting cell is at least one idiotypic peptide expressed by autoreactive T lymphocytes, preferably at least one idiotypic peptide derived from a CDR, more preferably CDR3, of an immunoglobulin or of a TCR chain, that may also contain CDR flanking segments.

Any of the techniques which are available in the art may be used to introduce the recombinant nucleic acid encoding the polypeptide into the antigen presenting cell. These techniques are collectively referred to as transfection herein and include, but are not limited to, transfection with naked or encapsulated nucleic acids, cellular fusion, protoplast fusion, viral infection, cellular endocytosis of calcium-nucleic acid microprecipitates, fusion with liposomes containing nucleic acids, and electroporation. Choice of suitable vectors for expression is well within the skill of the art. Antigen expression may be determined by any of a variety of methods known in the art, such as immunocytochemistry, ELISA, Western blotting, radioimmunoassay, or protein fingerprinting.

In an additional aspect of the present invention, a DNA vaccine is provided comprising a polynucleotide of the invention or an expression vector of the invention, both as described hereinabove.

In one embodiment, there is provided a DNA vaccine for prevention or treatment of cancer comprising a polynucleotide that encodes a polypeptide comprising at least one antigenic determinant of at least one TAA.

In another embodiment, there is provided a DNA vaccine for prevention or treatment of a disease caused by a pathogenic organism comprising a polynucleotide that encodes a polypeptide comprising at least one antigenic determinant of at least one pathogenic antigen.

The DNA vaccines may be constructed according to methods known in the art. Genes in plasmid expression vectors are expressed in vivo after intramuscular (i.m.) or subcutaneous (s.c.) injection and this expression stimulates an immune response against the plasmid-encoded proteins. The same or better effect is obtained replacing the plasmid by a viral vector.

In one embodiment, the DNA vaccine is a naked DNA vaccine. It may contain a plasmid DNA that contains the polynucleotide of the invention controlled by a cytomegalovirus (CMV) promoter. When the plasmid is introduced into mammalian cells, cell machinery transcribes and translates the gene. The expressed protein (immunogen) is then presented to the immune system where it can elicit an immune response. One method of introducing DNA into cells is by using a gene gun. This method of vaccination involves using pressurized helium gas to accelerate DNA-coated gold beads into the skin of the vaccinee.

DNA vaccines are capable of eliciting both strong humoral and cell-mediated immunity. Therefore DNA immunization represents a new approach for prevention (vaccination) and treatment (immune-based therapy) of infectious and neoplastic diseases.

In yet a further aspect of the invention, there is provided a cellular vaccine which comprises an antigen presenting cell of the invention as described hereinbefore. The antigen presenting cell is preferably a dendritic cell, but may also be a macrophage, a B cell and a fibroblast. The cells in the cellular vaccine may be autologous, allogeneic or xenogeneic cells.

The present invention provides cellular vaccines which comprise an antigen presenting cell that is capable of presenting at least one antigenic peptide comprising an epitope of at least one antigen and has the ability to induce potent CTL responses against the desired antigen(s). Vaccination, as used herein, refers to the step of administering the cellular vaccine to a mammal to induce such an immune response, for example, to prevent or treat a tumor or a disease caused by an infectious agent in a mammal.

The presentation of the at least one antigenic peptide by the APCs in the cellular vaccine can be achieved by transfecting the APCs with the polynucleotide of the invention, or by transducing the APCs with a virus encoding the polynucleotide of the invention or by incubating said antigen presenting cells with a polynucleotide encoding said at least one antigenic peptide.

In one embodiment, the invention provides a cellular vaccine for prevention or treatment of cancer wherein the antigen-presenting cell presents at least one peptide derived from at least one tumor-associated antigen.

In an additional aspect, the present invention provides a cellular vaccine for the prevention and/or treatment of a cancer comprising antigen-presenting cells which express a scβ₂m, i.e. a β₂-microglobulin linked through its carboxyl terminal to a polypeptide stretch that allows the anchorage of the β₂-microglobulin molecule to a cell membrane, wherein said polypeptide stretch consists of a bridge peptide which spans the whole distance to the cell membrane, said bridge peptide being linked to a sequence which can exert the required anchoring function, and wherein said cells have been pulsed with at least one antigenic peptide derived from at least one tumor associated antigen.

In still a further aspect, for the treatment of cancer it is envisaged by the present invention to encompass tumor cells transfected with a polynucleotide comprising a sequence encoding a scβ₂M, i.e. a polypeptide comprising a 2-microglobulin molecule that is linked through its carboxyl terminal to a polypeptide stretch that allows the anchorage of the β₂-microglobulin molecule to the cell membrane. The scβ₂m will enhance expression of the MHC class 1 molecules on the cell surface of the tumor cells.

In this aspect, it is known that tumor cells, which manifest impaired expression of MHC class I MHC molecules and are thus poorly immunogenic, can induce antitumor CTL activity upon transfection of MHC class I genes (Feldman and Eisenbach, 1991). The level of MHC class I expressed on the surface of tumor cells is a key factor, which governs immunogenicity of the tumor, and is amenable to genetic modification. It is evident from Table 2 hereinafter that the mere expression of scβ2m results in 3-4-fold enhancement in the level of H-2K^{k}. This effect can be harnessed to augment MHC class I expression by tumor cells. For example, tumor cells can be derived from the patient, transduced ex-vivo with a recombinant virus encoding membranal hβ₂m and expanded. Following their mitotic inactivation, transduced cells will be introduced back to the patient to serve as immunogens capable of eliciting a tumor-specific CTL response. This response may then target also unmodified tumor cells, provided they still express MHC class I molecules at a level sufficient for recognition by the armed effector CTLs.

In another embodiment, the invention provides a cellular vaccine for prevention or treatment of a disease caused by a pathogenic organism wherein the antigen-presenting cell presents at least one peptide derived from a pathogenic antigen.

In a further additional aspect, the present invention provides a cellular vaccine for the prevention and/or treatment of a disease caused by a pathogen comprising antigen-presenting cells which express a scβ₂m, i.e. a β₂-microglobulin linked through its carboxyl terminal to a polypeptide stretch that allows the anchorage of the β₂-microglobulin molecule to a cell membrane, wherein said polypeptide stretch consists of a bridge peptide which spans the whole distance to the cell membrane, said bridge peptide being linked to a sequence which can exert the required anchoring function, and wherein said cells have been pulsed with at least one antigenic peptide derived from at least one antigen of said pathogen.

The cellular vaccine may be administered subcutaneously, intradermally, intratracheally, intranasally, or intravenously. The cells may be suspended in any pharmaceutically acceptable carrier, such as saline or phosphate-buffered saline.

In still another aspect, the present invention provides a method of immunizing a mammal against a tumor-associated antigen comprising the step of: immunizing the mammal with an antigen-presenting cell which has been transfected with, or transduced with, or loaded with, a recombinant nucleic acid molecule comprising a sequence encoding a polypeptide comprising a β₂-microglobulin molecule that is linked through its carboxyl terminal to a polypeptide stretch that allows the anchorage of the β₂-microglobulin molecule to the cell membrane, and through its amino terminal to at least one antigenic peptide comprising a MHC class I epitope of at least one tumor-associated antigen, or with a cellular vaccine comprising said antigen presenting cell, wherein the mammal mounts a cytotoxic immune response against the at least one tumor-associated antigen, and wherein the antigen-presenting cell presents said at least one antigenic peptide.

In yet another aspect, the present invention provides a method of immunizing a mammal against a disease caused by a pathogenic organism comprising the step of: immunizing the mammal with an antigen-presenting cell which has been transfected with, or loaded with, a recombinant nucleic acid molecule comprising a sequence encoding a polypeptide comprising a β₂-microglobulin molecule that is linked through its carboxyl terminal to a polypeptide stretch that allows the anchorage of the β₂-microglobulin molecule to the cell membrane, and through its amino terminal to at least one antigenic peptide comprising a MHC class I epitope of a pathogenic antigen, or with a cellular vaccine comprising said antigen presenting cell, wherein the mammal mounts a cytotoxic immune response against the pathogenic antigen, and wherein the antigen-presenting cell presents said at least one antigenic peptide.

In still a further aspect, the present invention provides a method for the prevention and/or treatment of a cancer or of a disease caused by a pathogen which comprises administering to a patient in need thereof antigen-presenting cells which express a chimeric polypeptide comprising β₂-microglobulin linked through its carboxyl terminal to a polypeptide stretch that allows the anchorage of the β₂-microglobulin molecule to a cell membrane, wherein said polypeptide stretch consists of a bridge peptide which spans the whole distance to the cell membrane, said bridge peptide being linked to a sequence which can exert the required anchoring function, and wherein at least one antigenic peptide derived from at least one tumor associated antigen or from an antigen of said pathogen is exogenously loaded on said antigen-presenting cells, preferably in the grooves of the MHC complex formed by the association of the chimeric polypeptide with the endogenous MHC molecule component.

In yet still a further aspect, the present invention provides pharmaceutical compositions. In one embodiment, the composition comprises as an active ingredient at least one polynucleotide or an expression vector of the invention, and a pharmaceutically acceptable carrier. The polynucleotide may comprise a sequence encoding a polypeptide comprising at least one antigenic peptide derived from at least one tumor associated antigen, or at least one antigenic peptide derived from a pathogenic antigen. In another embodiment, the pharmaceutical composition comprises as an active ingredient at least one antigen-presenting cell of the invention, and a pharmaceutically acceptable carrier.

Good cancer vaccines should induce a protective CTL response directed at MHC class I peptides derived from TAAs. The pivotal APC in CTL priming is the dendritic cell (DC), which has indeed been widely utilized in the design of cancer vaccines. In particular, DCs are attributed a critical role in DNA immunization, and direct presentation of peptides derived from expression of genetic material internalized by DCs is considered a major route for CTL induction. While magnitude of a CTL response correlates with density of specific MHC-peptide complexes on the APC surface, many TAA peptides have low affinity for the class I molecule and are presented at sub-optimal densities. Combined with the limiting expression level normally achieved following administration of non-replicating DNA, DNA immunization against TAAs usually falls short from achieving the anticipated effect.

The double-chimeric β₂m (dcβ₂m) polypeptide design of the present invention creates an entirely novel MHC class I entity, which offers a great advantage over current strategies as a means to augment CTL induction. The membrane anchorage of the β₂m molecule can be achieved by covalently linking to its carboxyl terminal a peptide bridge, which spans the whole distance to the cell membrane, and is supplemented by an anchoring sequence such as the transmembrane and cytoplasmic domains derived from another cell surface protein. Following dissociation of β₂m-linked peptide from the α chain, this design is expected to prevent detachment of the β₂ m/peptide from the cell membrane. It has been found in accordance with the present invention that membrane anchorage immensely increases the local concentration of dcβ₂m in the cell membrane, and allow rapid re-formation or de-novo formation of the specific MHC class I complex upon peptide dissociation. This will significantly prolong the actual half-life of the complex and increase its membranal level.

Chimeric β₂m polypeptides having a sole antigenic peptide linked to their amino terminal, which are provided exogenously, have been shown to associate with α chains on the cell surface and to form full MHC class I complexes (Uger and Barber, 1998′ Tafuro et al., 2001; Uger et al., 1999; White et al., 1999). According to the present invention, it is also assumed that re-association will take place on the cell membrane but obeying kinetics of lateral diffusion. Furthermore, but not less important, the high local peptide concentration, the membranal form of β₂m and the anticipated proteasome- and TAP-independence according to the invention, are all expected to render initial assembly of the specific, intact MHC class I complex in the ER highly favorable, compared with assembly involving processing and transportation of conventional, cytosolic peptides.

As used herein, the term “double-chimeric β₂-microglobulin” (dcβ₂m) refers to a molecule of β₂m having at least one epitope/antigenic peptide bound to the amino terminal and an anchor domain bound to the carboxyl terminal, wherein said anchor domain is composed of a polypeptide stretch consisting of a bridge peptide, which spans the whole distance to the cell membrane, and a peptide sequence that allows the anchorage of the β₂-microglobulin molecule to the cell membrane. The term “single-chimeric β₂-microglobulin” (scβ₂m), when used herein, refers to a molecule of β₂m having only the anchor domain, as defined above, but no antigenic peptide at the amino terminal.

The realization that vaccination with naked DNA results in long-lasting protein expression and stimulation of specific humoral and cellular immune responses, has made a large impact in the field of vaccine (see Gurunathan et al., 2000 for review). Numerous studies, which have shown that DNA vaccines induce potent MHC class I-restricted CTL responses against TAAs, have suggested that this modality may be particularly useful for the treatment of cancer, and have prompted the development of a variety of DNA vaccine strategies (see review by Benton and Kennedy, 1998). First human trials of cancer DNA vaccines have been initiated, but it is too early to evaluate their efficacy. There is compelling evidence that a CTL response following DNA administration can be induced by directly transfected DCs (Porgador et al., 1998), although other mechanisms, such as direct transfection of somatic cells or cross presentation by DCs, are also considered.

According to the present invention, direct delivery of the dcβ₂m polypeptide produced by DCs, which express the introduced gene, to surface MHC class I molecules for peptide presentation is expected to results in considerable enhancement in peptide level, and hence, in vaccine efficacy, compared with that achieved by conventional antigen processing and presentation.

The present invention thus provides a novel and broadly-applicable strategy for efficient induction of antigen-specific CTLs, which is based on the ability of dcβ₂m to markedly enhance presentation of antigenic peptides. The CTL response may be optimized by a regimen of two or more booster administrations. Cocktails of two or more CTL inducing peptides are employed to optimize epitope and/or MHC class I restricted coverage.

For the purposes of the present invention, the biochemical and immunological properties associated with this mode of presentation are first explored in vitro in transfected cell lines, and its in vivo function is then assessed in a mouse melanoma tumor model, applying transfected APC cell lines, naked DNA immunization and adoptive transfer of syngeneic APCs from transgenic mice.

Defining various parameters, which govern expression of dcβ₂m, and establishing its actual potential as a tumor vaccine in a mouse model are expected to pave the way for the design of a novel modality of human cancer vaccines. The most suitable effector cells for this purpose are autologous DCs, which can be relatively easily transduced to express foreign genes (Hadzantonis and O'Neill, 1999; Bubenik, 2001).

The inability to present low affinity peptides at densities required for potent activation of the entire repertoire of peptide-specific CTL clones is considered a major obstacle in many of the current protocols, which aim at producing DC-based cancer vaccines. According to the present invention, it has been shown that the dcβ₂m-based constructs increases the apparent affinity of the peptide to the MHC molecule and, thus, the dcβ₂m-mediated presentation on DCs should allow TAA-derived peptides with limiting affinity for the restricting MHC class I product to be presented by the DCs at sufficiently high density. This is one of the expected advantages of the present invention in comparison to previously proposed approaches for the development of cancer vaccines based on dendritic cells.

Some TAAs are expected to play an active part in the induction of central tolerance in the thymus, thus allowing only CTLs of low avidity to mature (Gilboa, 1999). These may include TAAs which are classified as differentiation antigens (for example MART-1/Melan A, gp100 and tyrosinase), and, probably to a lesser extent, normal gene products with highly restricted tissue distribution (such as MAGE, BAGE and GAGE). The strategy of the present invention can be efficient in activating such low avidity CTLs.

Tumors often evade the immune system by reduction in MHC class I peptide presentation to CTLs by downregulation of either components of the proteasome complex or TAP (for review see Benton and Kennedy, 1998). Enhancement of TAA peptide presentation by such tumors following gene delivery activates CTLs, which can respond also to non-modified tumor cells (Sherritt et al., 2001), provided the density of class I tumor-associated epitopes exceeds a functional threshold of these CTLs. Hence, dcβ₂m or scβ₂m are expected to induce CTLs not only in professional APCs as dendritic cells but, in certain cases, also when expressed in tumor cells.

In the present invention, we have converted β₂m to a membranal protein as a novel backbone for potentiating maximal MHC-I presentation of genetically linked peptides, and demonstrated its in-vivo efficacy as the core component of cancer vaccines. We have harnessed the chimeric receptor approach for the generation of a novel class of genetic cancer vaccines, which incorporate the remarkable presentation capacity conferred by membranal β₂m with the potential ability to induce full DC maturation and reverse, or induce, CTL tolerance through selected TLR signaling elements. The dcβ₂m prompts exceptionally efficient peptide presentation on MHC-I molecules, by: directly targeting β₂ m/peptide to the ER through the leader peptide, uncoupling presentation from proteasomal degradation and TAP-mediated translocation; avoiding the need for N-terminal peptide trimming at the ER; facilitating full MHC-I complex assembly; yielding abundance of peptide available for de-novo complex formation at the cell membrane (Margalit et al., 2003).

Stimulation of different TLRs on APCs can skew the ensuing response towards activation of the Th1 arm, the Th2 and humoral arm or towards immunosuppression, mainly via Treg induction. For example, whereas engagement of TLR4 on human DCs promotes the production of Th1-inducing cytokines, stimulation of TLR2 on the same cells produces conditions that antagonize Th1 cells and favor a Th2 response (Re and Strominger, 2001). In addition, it was found that TLR2 induces Tregs (Liu et al., 2006). Therefore, an TLR2-β₂m construct according to the invention can potentially suppress the immune response against selected targets. These discrete and antagonistic pathways can be harnessed by appropriate adjuvants to modulate the response to vaccines (Pulendran, 2004). The engraftment of a particular TLR activation domain onto β₂m can achieve the same effect. For example, while TLR4 can be incorporated in vaccines against cancer or infectious agents, TLR2-β₂m in conjunction with a relevant self-peptide can be used to shift a pathogenic Th1 response into an antagonizing, and therefore beneficial, Th2 response. This design is applicable in multiple sclerosis (MS), insulin-dependent diabetes mellitus (IDDM) and other autoimmune diseases, which are largely dominated by Th1 activity. Alternatively, introduction of such vaccines to immature rather than to mature DCs. can exert an antigen-specific tolerizing effect (Mahnke et al., 2003).

Thus, APC activation domains linked to β₂m, with or without CTL epitopes, can be used in diverse contexts according to the invention.

In one embodiment, the invention relates to the induction of CTLs against cancer and infectious agents, as described above.

In another embodiment, the invention relates to the induction of CTLs against other cellular targets including benign cells, which inflict damage as a result of dysregulated activity or the production of harmful substances. For example, IgE-producing B cells play a key role in the initiation and maintenance of allergic diseases and asthma. MHC-I-binding peptides derived from either the heavy or the light chain of IgE and, particularly, from the constant region of IgE (Fcε), can be used to target CTLs specifically to this B cell subset (Chen et al., 2005). Analyzing-human Fcε with two prediction algorithms for MHC binding (Rammensee et al., 1999; Parker et al., 1994), we identified several HLA-A2 binding peptide candidates. Among these are: SEQ ID NO: 65, SLNGTTMTL (46); SEQ ID NO: 66, TLPATTLTL (53); SEQ ID NO: 67, DLAPSKGTV (240); SEQ ID NO: 68, TLSGHYATI (60); SEQ ID NO: 69, TITCLVVDL (233); SEQ ID NO: 70, YATISLLTV (65); SEQ ID NO: 71, ELASTQSEL (168); SEQ ID NO: 72, GTLTVTSTL (273); SEQ ID NO: 73, ALMRSTTKT (306); SEQ ID NO: 74, NIPSNATSV (16); SEQ ID NO: 75, ATSVTLGCL (21); SEQ ID NO: 76, TMTLPATTL (51); SEQ ID NO: 77, FTPPTVKIL (107); SEQ ID NO: 78, SVQWLHNEV (352); SEQ ID NO: 79, FICRAVHEA (399). The numbers within brackets denote the starting position of the peptide at the IgE constant region sequence (Fcε). Such peptides can be linked according to the invention, for example, to TLR4-β₂m. The resulting vaccines are expected to induce CTLs against IgE-expressing B cells in an immunostimulatory environment, geared to break potential peripheral tolerance to IgE.

In a further embodiment, the invention relates to autoreactive T cells that constitute another potential target for such vaccines. The TCR variable (V) regions of encephalitogenic T cells in MS and animal models of this disease often utilize conserved Vα and Vβ gene segments, which can be used as potential targets for vaccines against this disease (Howell et al., 1989). Shared MHC-I-binding idiotypic peptides from TCRs of auto-aggressive T cells can be appended to the N-terminus of β2m and exploited as a means to eliminate or suppress these cells. This use of idiotypic peptides is in essence similar to their use in β2m-based vaccines against lymphoid malignancies, which express antigen receptor genes.

In another embodiment, the invention relates to autoreactive CD8 CTLs, which are involved in the pathogenesis of autoimmune diseases such as MS and IDDM (Liblau et al., 2002; Steinman, 2001). Peptides from the insulin B chain (Wong et al., 1999) and GAD65 (Quinn et al., 2001) are implicated in the initiation of IDDM in NOD mice while CD8 T cell clones specific to peptides from myelin basic protein (MBP) (Huseby et al., 2001) and myelin oligodendrocyte glycoprotein (MOG) (Sun et al., 2001) are encephalitogenic in mouse models for MS. A self MHC-I binding peptide associated with an autoimmune disease can be linked to TLR2-β₂m. When expressed by mature DCs, such constructs can potentially suppress the auto-reactive CD8 T cell clones via the production of Th2 cytokines. Alternatively, expressing these constructs by immature DCs can induce antigen-specific suppressor CD8 T cells (Cortesini et al., 2001).

In another further embodiment, the invention relates to the suppression of allo-reactive CD8 T cells, which is a primary goal in the treatment of transplant rejection and graft-versus-host disease (GVHD). CD8 T cell reactivity in these two conditions is in large directed against the foreign HLA-I alleles in a peptide-nonspecific manner. Thus, peptide-less β₂m can be provided with the activation domain of TLR members, such as TLR2, which promote a Th2, rather than a Th1 response. Expressing the resulting construct in donor APCs (in the case of transplant rejection) or in APCs of the recipient (in GVHD) can diminish pathogenesis mediated by the alloreactive CD8 T cells. Alternatively, introducing TLR-β₂m to immature DCs can result in a tolerogenic effect, suppressing the same pathogenic CD8 T cell clones.

In addition to the above mentioned aspects, the present invention provides a polynucleotide comprising a sequence encoding a polypeptide that is capable of high level presentation of antigenic peptides on antigen-presenting cells, wherein the polypeptide comprises a β2-microglobulin molecule that is linked through its carboxyl terminal to a polypeptide stretch that allows the anchorage of the β2-microglobulin molecule to the cell membrane, and through its amino terminal to at least one antigenic peptide comprising an MHC class I epitope, and wherein said polypeptide stretch consists of a bridge peptide that spans the whole distance to the cell membrane, said bridge peptide is linked to the full or partial transmembrane and/or cytoplasmic domains of a molecule selected from the group consisting of a toll-like receptor (TLR) polypeptide, a CD40 polypeptide, and a TLR polypeptide and a CD40 polypeptide fused in tandem so that their biological functions are preserved.

In one preferred embodiment, the bridge peptide is linked to the full or partial transmembrane and/or cytoplasmic domains of a toll-like receptor (TLR) polypeptide such as a TLR 3, 4, 5, 7, 8 or 9 polypeptide, preferably TLR4, in which case a Th1 type of response and subsequent CTL induction is mediated by engagement of said TLR members, which trigger the production of IL-12 (p70) and interferon α, in addition to other cytokines. In another embodiment the signal transduction element is a TLR polypeptide such as TLR2, TLR1 or TLR6, capable of mediating a Th2 response, and in a preferred embodiment it is TLR2.

In another preferred embodiment, the bridge peptide is linked to the full or partial transmembrane and/or cytoplasmic domains of a CD40 polypeptide.

In another embodiment, the bridge peptide is linked to the full or partial transmembrane and/or cytoplasmic domains of a chimera formed by TLR and CD40 polypeptides fused in tandem so that their biological functions are preserved.

All the embodiments described hereinabove related to vectors, cells, DNA vaccines, cellular vaccines, methods of immunization, and antigenic peptides derived from tumor-associated antigens, or from a pathogen selected from the group consisting of a bacterial, viral, fungal and parasite antigen, or from a membrane bound IgE molecule antigen, are suitable also for the particular embodiments wherein the bridge peptide is linked to the full or partial transmembrane and/or cytoplasmic domains of a molecule selected from the group consisting of a toll-like receptor (TLR) polypeptide, a CD40 polypeptide, and a TLR polypeptide and a CD40 polypeptide fused in tandem, and the antigenic peptide is not a peptide related to an autoimmune disease.

In another embodiment, the present invention provides a polynucleotide comprising a sequence encoding a polypeptide that is capable of high level presentation of antigenic peptides on antigen-presenting cells, wherein the polypeptide comprises a β2-microglobulin molecule that is linked through its carboxyl terminal to a polypeptide stretch that allows the anchorage of the β2-microglobulin molecule to the cell membrane, and through its amino terminal to at least one antigenic peptide comprising an MHC class I epitope associated with an autoimmune disease, and wherein said polypeptide stretch consists of a bridge peptide that spans the whole distance to the cell membrane, said bridge peptide is linked to the full or partial transmembrane and/or cytoplasmic domains of a molecule selected from the group consisting of a toll-like receptor (TLR) polypeptide, a CD40 polypeptide, and a TLR polypeptide and a CD40 polypeptide fused in tandem so that their biological functions are preserved.

According to this embodiment, the antigenic peptide may be associated with an autoimmune disease such as, but not being limited to, insulin-dependent diabetes mellitus (IDDM), multiple sclerosis (MS), systemic lupus erythematosus (SLE), rheumatoid arthritis, several forms of anemia (pernicious, aplastic, hemolytic), thyroiditis, and uveitis.

In one embodiment, the polynucleotide of the invention comprises a sequence encoding a polypeptide comprising a TLR domain or TLR and CD40 polypeptides fused in tandem so that their biological functions are preserved in which the at least one autoimmune disease associated antigenic peptide comprising a MHC class I epitope is linked to the β₂-microglobulin amino terminal directly. In another embodiment, the at least one autoimmune related antigenic peptide is linked to the β₂-microglobulin amino terminal through a peptide linker. The bridge peptide is preferably a peptide of SEQ ID NO: 1. The invention further comprises a vector comprising said polynucleotide and cells which contain said vector and express said polypeptide. The cells are preferably immune cells selected from T helper cells (CD4⁺), cytotoxic T lymphocytes (CD8⁺) and natural killer (NK) cells, which are capable of recognizing and binding to harmful autoreactive T cells causing the autoimmune disease with which the antigenic peptide is associated, and causing their elimination or inactivation. Reference is made to copending U.S. application Ser. No. 10/297,060, herewith incorporated by reference in its entirety as if fully disclosed herein.

The present invention further relates to a method for the prevention and/or treatment of an autoimmune disease which comprises administering to a patient in need thereof autologous T cells which express a chimeric polypeptide comprising a β2-microglobulin molecule that is linked through its carboxyl terminal to a polypeptide stretch that allows the anchorage of the β2-microglobulin molecule to the cell membrane, and through its amino terminal to at least one antigenic peptide comprising an MHC class I epitope associated with said autoimmune disease, and wherein said polypeptide stretch consists of a bridge peptide that spans the whole distance to the cell membrane, and said bridge peptide is linked to the full or partial transmembrane and/or cytoplasmic domains of a molecule selected from the group consisting of a toll-like receptor (TLR) polypeptide, a CD40 polypeptide, and a TLR polypeptide and a CD40 polypeptide fused in tandem so that their biological functions are preserved.

The invention further relates to a method for the prevention and/or treatment of an autoimmune disease which comprises administering to a patient in need thereof autologous T cells which express a chimeric polypeptide comprising (a) a β2-microglobulin molecule that is linked through its carboxyl terminal to a polypeptide stretch that allows the anchorage of the β2-microglobulin molecule to the cell membrane, wherein said polypeptide stretch consists of a bridge peptide that spans the whole distance to the cell membrane, and said bridge peptide is linked to (b) the full or partial transmembrane and/or cytoplasmic domains of a signal transduction element capable of activating T cells selected from the group consisting of a toll-like receptor (TLR) polypeptide, a CD40 polypeptide, and a TLR polypeptide and a CD40 polypeptide fused in tandem so that their biological functions are preserved, and wherein one or more antigenic peptides related to said autoimmune disease are exogenously loaded in the grooves of the MHC complex formed by the association of component (a) with the endogenous MHC molecule component. The antigenic peptides may be exogenously supplied by incubation of the cells with one or more peptides or proteins associated with said autoimmune disease.

In another aspect, the present invention provides a cell which expresses a β2-microglobulin molecule that is linked through its carboxyl terminal to a polypeptide stretch that allows the anchorage of the β2-microglobulin molecule to the cell membrane, wherein said polypeptide stretch consists of a bridge peptide that spans the whole distance to the cell membrane, and said bridge peptide is linked to the full or partial transmembrane and/or cytoplasmic domains of a signal transduction element capable of activating T cells selected from the group consisting of a toll-like receptor (TLR) polypeptide, a CD40 polypeptide, and a TLR polypeptide and a CD40 polypeptide fused in tandem so that their biological functions are preserved. The cell is preferably an immune cell selected from T helper cells (CD4⁺), cytotoxic T lymphocytes (CD8⁺) and natural killer (NK) cells, capable of recognizing and binding to harmful T cells and causing their elimination or inactivation. The invention further provides an immune cell which expresses said polypeptide and binds to, and eliminates, alloreactive cells causing transplant rejection. The immune cell is preferably a cytotoxic T lymphocyte (CTL).

In another aspect, a method is provided for the prevention and/or treatment of graft-versus-host disease (GVHD) which comprises administering to a patient in need thereof at suitable times autologous T cells which express a β2-microglobulin molecule that is linked through its carboxyl terminal to a polypeptide stretch that allows the anchorage of the β2-microglobulin molecule to the cell membrane, wherein said polypeptide stretch consists of a bridge peptide that spans the whole distance to the cell membrane, and said bridge peptide is linked to the full or partial transmembrane and/or cytoplasmic domains of a signal transduction element capable of activating T cells selected from the group consisting of a toll-like receptor (TLR) polypeptide, a CD40 polypeptide, and a TLR polypeptide and a CD40 polypeptide fused in tandem so that their biological functions are preserved.

In another embodiment, a method is provided for the prevention and/or treatment of host-versus-graft reaction which comprises administering to a patient in need thereof at suitable times donor T cells which express a β2-microglobulin molecule that is linked through its carboxyl terminal to a polypeptide stretch that allows the anchorage of the β2-microglobulin molecule to the cell membrane, wherein said polypeptide stretch consists of a bridge peptide that spans the whole distance to the cell membrane, and said bridge peptide is linked to the full or partial transmembrane and/or cytoplasmic domains of a signal transduction element capable of activating T cells selected from the group consisting of a toll-like receptor (TLR) polypeptide, a CD40 polypeptide, and a TLR polypeptide and a CD40 polypeptide fused in tandem so that their biological functions are preserved.

The cell for use in the present invention may be any type of antigen-presenting cell or any type of immune cell (a cell involved in the immune system) or a precursor thereof, such as, but not limited to, lymphocytes including B cells, T cells, natural killer T (NKT) cells, plasma cells, granulocytes, neutrophils, platelets, mast cells, macrophages and dendritic cells. The T cells are preferably T helper cells (CD4⁺), cytotoxic T lymphocytes (CD8⁺), natural killer (NK) cells, and T regulatory cells (Treg; CD4⁺CD25⁺). Some of these cells are professional antigen-presenting cells such as dendritic cells, macrophages and B cells. The invention encompasses such cell types (e.g., dendritic cells, macrophages etc) both in the mature and immature or precursor stages.

In the present invention we harness the chimeric receptor approach for the generation of a novel class of genetic cancer vaccines, which incorporate the remarkable presentation capacity conferred by membranal β₂m with the potential ability to induce full DC maturation and reverse CTL tolerance through selected TLR signaling elements Peptide-based immunogens have short half-lives in vivo, and presentation of peptides pre-loaded onto DCs terminates once they dissociate from the MHC-I heterodimer. In contrast, genetic vaccines drive long-term expression of the immunogen. This point is of crucial importance in vivo, since the time elapsing between binding of synthetic peptides and engagement with CTL precursor in the lymph node may well exceed the peptide life span at the MHC-I binding groove, especially for low-to-medium affinity peptides (Wang and Wang, 2002). The high density of peptide, which can be achieved following peptide pulsing of cells, can be paralleled, and even surpassed, by our genetic design, even when we use a non-replicating vector (Margalit et al., 2003). Furthermore, the monomorphic nature of β₂m renders it a truly universal platform for expressing peptides restricted by all HLA-I alleles or, when expressed without linked antigenic peptide, for loading synthetic peptides (Berko et al., 2005). APCs expressing TAA peptides linked to membranal β₂m induce superior anti-tumor immunity than peptide-saturated APCs in a mouse melanoma model (Margalit et al., 2006).

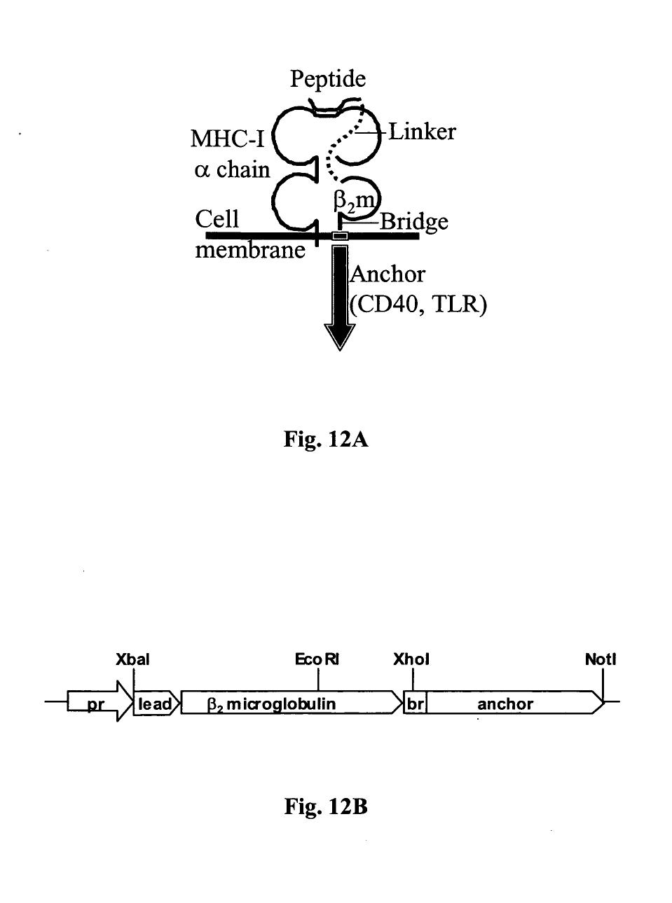

The present invention focuses on the development of a novel class of genetic vaccines, based on this membranal β₂m platform, which exploit the chimeric receptor strategy. These vaccines combine optimal presentation of the genetically linked peptide with the ability to stimulate DC maturation and Treg suppression mediated by TLR signaling domains, incorporated as the β₂m anchor portion (see FIG. 12).

Cumulative data from clinical studies in patients with solid tumors reveal very low rate of clinical response and underscore the need for new approaches in the design of cancer vaccines (Rosenberg et al., 2004). The coupling, in a single gene product, of a potent and sustained antigenic stimulus with signals required for curtailing Treg activity and for DC activation and maturation, is unprecedented, and opens a fundamentally new avenue in vaccine design. This approach precludes the use of potentially toxic adjuvants, as proper triggering is expected to be confined to DCs expressing the gene and occur in the absence of microbial-derived stimuli. Such an achievement not only allows the effective and safe tackling of immunological obstacles imposed by many cancers, but also greatly simplifies vaccine production and delivery.

Recent findings ascribe a critical role for Tregs in suppressing tumor-specific T cells. Treg depletion or the overruling of their regulatory effects by counter-acting adjuvants, have consequently become major challenges in the development of protocols for cancer immunotherapy. While lymphocyte depletion has a positive effect, it reduces the number of tumor-specific T cell precursors available for induction, so that more selective methods are required. TLR agonists employed as adjuvants strongly promote DC maturation. However, besides associated side-effects, it is the persistence of their induced signaling, which is necessary for breaking Treg-mediated CTL tolerance (Yang et al., 2004). Such persistent signaling is hard to produce in regular immunization regimens. The integration, through a single polypeptide product, of the constitutively active phenotype induced by the intracellular domain of TLR4 (Medzhitov et al., 1997, Cisco, et al., 2004) with MHC-I stabilization and excellent peptide presentation conferred by membrane-anchored β₂m, represents a novel multifunctional vaccine modality and is one of the main objectives of this invention.

Sensitization of tissue DCs by pathogen-derived signals triggers their migration to the draining lymph node. In order to complete their differentiation program and gain CTL priming capacity, these DCs require ‘licensing’ by antigen specific CD4⁺Th1 cells. This depends on the engagement of CD40 ligand (CD40L) on the Th1 cell with the CD40 receptor on the DC. Th1 dependence can be overcome through CD40 ligation by agonistic anti-CD40 antibodies or soluble CD40L. CD40 cross-linking has been shown to bypass Th1 help, suppress tolerance and enhance CTL-mediated anti-tumor immunity in several model systems. Pharmacological cross-linking of a genetically engineered CD40 activation domain was recently reported to improve the immunostimulatory properties of DCs and augment anti-tumor CTL immunity. Many peptide-based vaccines assessed in animal models and in clinical studies do not provide CD4 T cell epitopes and fail to recruit adequate Th1 help for CTL activation. In our preliminary studies in mouse APCs, stable expression of CD40 activation domain fused with β₂m resulted in a new phenotype (data not shown).

Coupling the antigenic moiety to CD40 signaling pathway offers a new means for the elicitation and amplification of Th1-mediated signaling by cancer vaccines. The ability to combine functions conferred by both TLR and CD40 domains, along with superb TAA peptide presentation, is expected to maximize the ability of the resulting vaccine to drive DC differentiation along the desired pathway. This can be achieved according to the invention through the assembly of a single tripartite gene, in which the TLR and the CD40 portion are fused in tandem to peptide-bearing β₂m, so that the biological function of both is preserved and all the immunological effects are imparted in parallel. These vaccines will be compatible with all clinical in-vivo and ex-vivo protocols for gene delivery into human DCs. The use of a single gene should render all genetic manipulations required for vaccine construction considerably simpler, and ensure the delivery of all components to the same DCs.

We have constructed in the present invention a membrane-anchored derivative of β₂ microglobulin (β₂m), the monomorphic MHC-I light chain, as a novel platform for genetic cancer vaccines. When expressed by antigen-presenting cells (APCs), this design stabilizes MHC-I, potentiates remarkable MHC-I presentation of N-terminally linked peptides and confers effective anti-tumor immunity in a mouse melanoma model. Membrane-anchored β₂m carrying TAA-derived peptides is an ideal backbone for engrafting intracellular signaling domains from DC activation receptors, thus providing CTL epitopes with a built-in adjuvant component. This design should enhance the potency of CTL priming and, consequently, the overall immunogenicity and clinical efficacy of such cancer vaccines.

In a particular embodiment, we have chosen to incorporate, as the adjuvant moiety, the transmembrane and cytosolic (tm+cyt) portion of TLR4 for two reasons: first, expression of truncated TLR4 devoid of its ectodomain results in constitutive activation of APCs; second, persistent TLR4 signaling has been implied in the reversal of CTL tolerance sustained by tumor-specific Tregs. In a series of preliminary in-vitro experiments, we monitored function of the tm+cyt portion of either mouse or human TLR4 when fused to the C-terminus of β₂m. Using semi-quantitative RT-PCR analysis for cytokine production, we showed in the examples below that a peptide-less configuration confers a constitutively activated phenotype on transfected human THP-1 monocytes and mouse RAW264.7 macrophages. We similarly demonstrated that RAW264.7 cells transfected with tripartite peptide-β₂m-TLR4 constructs are constitutively activated and at the same time stimulate a mouse T cell hybridoma in a peptide-specific manner. Finally, ex-vivo propagated, immature human DCs exhibit elevated expression of co-stimulatory molecules following transfection with in-vitro transcribed mRNA encoding peptide-β₂m-TLR4.

Our findings confirm that the integrated TLR4 signaling domain is functional, while the ability of the extracellular peptide-β₂m segment to pair with MHC-I heavy chain is preserved. Comparative studies evaluating anti-tumor efficacy of these vaccines in different in-vivo and ex-vivo settings are underway and will show the advantages of the invention.

The invention will now be illustrated by the following non-limiting examples.

### EXAMPLES

### Materials and Methods for Examples 1-5

(i) Cells. MD45 is an H-2D^{b}-allospecific mouse H-2^{k/d} CTL hybridoma of BALB/c origin (Faufmann et al., 1981). RMA-S is a mutant cell line derived from the C57BL/6 lymphoma RMA (H-2 b), which has defects in peptide presentation by class I MHC molecules due to loss of functional expression of the TAP component TAP-2. These cells can be loaded exogenously with high levels of MHC class I compatible peptides. RMA/OVA and RMA-S/OVA are clones of these two cells transfected with the full-length chicken ovalbumin gene. B3Z is an H-2K^{b}-restricted, OVA₂₅₇₋₂₆₄-specific CTL hybridoma, harboring the NFAT-LacZ reporter gene (Sanderson and Shastri, 1994), and is a gift from Dr. N. Shastri, University of California, Berkeley. Three clones of the mouse melanoma B16, a spontaneously-arising melanoma of C57BL/6 origin are used: F10.9 is a spontaneously metastasizing clone of the B16-F10 line, KI is an H-2K^{b} transfectant of F10.9 (Porgador et al., 1989) and MO5 is a chicken ovalbumin-transfected variant of the B16 melanoma. C57BL/6-derived T cell Line A, reactive with TRP-2 peptide 181-188 (TRP-2₁₈₁₋₁₈₈) (Bloom et al., 1997), is available from Dr. J. Yang, NCI, NIH, USA.

(ii) Antibodies. Fab13.4.1 is a Fab fragment specific to Ha₂₅₅₋₂₆₂ in the context of K^{k}, and was a gift from Dr. J. Engberg, University of Copenhagen (Andersen et al., 1996). AF3-12.1 is an anti-K^{k} mAb (Pharmingen). BM-63 is an anti-human β₂m mAb (Sigma). 20.8.4 is an anti-H-2K^{b} mAb. 28-14-8 is an anti-H-2 Db mAb. 25-D1.16 is specific to the complex H-2K^{b}/OVA₂₅₇₋₂₆₄ (Porgador et al., 1997). These latter antibodies are available from Dr. L. Eisenbach, Weizmann Institute of Science, Rehovot, Israel.

(iii) DNA transfection. 5-10×10⁶ RMA-S cells in 0.8 ml were mixed in 4 mm sterile electroporation cuvette (ECU-104, EquiBio, Ashford, UK) with 10-20 μg DNA of the constructed plasmid and placed on ice. Transfection was performed by electroporation using Easyject Plus electroporation unit (EquiBio, Ashford, UK) at 350V, 750 μF. Cells were resuspended in fresh medium and cultured for 24-48 hours in 96-well plates prior to addition of the selecting drug (1 mg/ml G418). Resistant clones were first expanded in 24-well plates and analyzed for expression of the introduced gene by FACS.

(iv) FACS analysis. Cells were stained with indicated antibodies according to standard procedures and were subjected to flow cytometry analysis. 10⁶ cells were washed with phosphate-buffered saline (PBS) containing 0.02% sodium azide and incubated for 30 minutes on ice with 100 μl of the anti-human β₂m mAb (Sigma) at 10 μg/ml or the same concentration of a control antibody (or no antibody). Cells were then washed and incubated on ice with 100 μl of 1:100 dilution of goat anti-mouse IgG (FAB specific)-FITC conjugated polyclonal antibody (Sigma) for 30 minutes. Cells were washed and resuspended in PBS and analyzed by a FACSCalibur (BD Biosciences, Mountain View, Calif.). Statistical analysis was performed with the FACSCalibur CellQuest software. Quantitative analysis of cell surface antigens was performed with QIFIKIT (DAKO, Carpinteria, Calif.) according to the manufacturer's instructions.

(v) Cell stimulation assay. Cells at 5×10⁵ cells/ml were incubated overnight in 96-well plates in the presence of 5 μg/ml antibody (immobilized overnight and washed 3 times in PBS) or with target cells at 5×10⁵ cells/ml. Total volume: 0.1 ml.

(vi) In-cell X-Gal staining. Cells in 96-well plates were washed twice with PBS and fixed with 0.25% glutaraldehyde for 15 min, washed 3 times in PBS, incubated for 4 hours with 100 μl of X-Gal solution {0.2% X-Gal, 2 mM MgCl₂, 5 mM K₄Fe(CN)₆.3H2O, 5 mM K₃Fe(CN)₆ in PBS} and scored under the microscope for blue staining.

(vii) Immunization of mice. Immunization was carried out with peptide loaded RMA-S cells, RMA-S, RMA-S/OVA and RMA/OVA and OVA₂₅₇₋₂₆₄-dcβ₂m-expressing RMA-S transfectants (Y314-7 and Y317-2): RMA-S cells were incubated at 2×10⁶ cells/ml for 2 hours with 200 μg/ml of OVA₂₅₇₋₂₆₄. Mice were immunized twice i.p. with 2×10⁶ irradiated (50 Gy) cells, at 10 day intervals.

(viii) Cytotoxicity assay. Ten days after last immunization spleens were removed and single cell suspension were prepared. Splenocytes were restimulated with irradiated, mitomycin-C-treated tumor cells or target cells. Restimulated lymphocytes were maintained for another 4 days. Viable lymphocytes were separated on Lympholyte-M gradient (Cendarlane, Ontario, Canada) and resuspended at 5×10⁶/ml with lymphocyte medium. Lymphocytes were mixed at different ratios (1:100, 1:50, 1:25 and 1:12.5 target to effector) with ³⁵S-methionine-labeled target cells (tumor cells or peptide-presenting cells). CTL assays were carried out following standard procedures.

### Example 1

### Expression of dcβ₂m Designed for T Cell Re-Programming

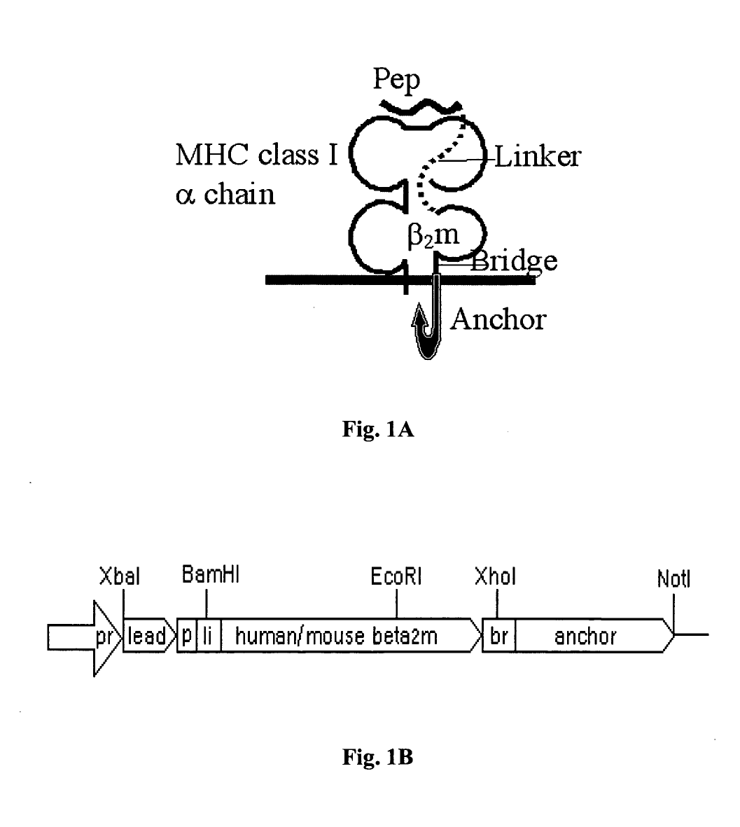

The general schemes of genetic constructs encoding dcβ₂m and of the polypeptide product associated with an MHC class I heavy chain are illustrated in FIG. 1. Table 1 summarizes all different single and double chimeric β₂m expression plasmids generated in this system as well in the tumor experimental system, which will be described below.

TABLE 1
Double and single chimeric β₂m constructs
and transfected clones expressing them

| | | | | Cell | |
|---|---|---|---|---|---|
| Peptide | Allele | β₂m | Anchor | (H-2) | Clone |
| Ha₂₅₅₋₂₆₂ | H-2K^{k} | human | none | MD45(k/d) | 840-7 |
| Ha₂₅₅₋₂₆₂ | H-2K^{k} | human | CD3 ζ | MD45 | 427-24 |
| NP₅₀₋₅₇ | H-2K^{k} | human | CD3 ζ | MD45 | 425-44 |
| Insulin | H-2K^{d} | mouse | CD3 ζ | MD45 | 829S-36 |
| B15-23 | | | | | |
| OVA₂₅₇₋₂₆₄ | H-2K^{b} | mouse | H-2K^{b} | RMA-S(b) | Y314-7 |
| OVA₂₅₇₋₂₆₄ | H-2K^{b} | human | H-2K^{b} | RMA-S | Y317-2 |
| TRP-2₁₈₁₋₁₈₈ | H-2K^{b} | mouse | H-2K^{b} | RMA-S | Y313-10 |
| TRP-2₁₈₁₋₁₈₈ | H-2K^{b} | human | H-2K^{b} | RMA-S | Y318-7 |
| none | — | human | CD3 ζ | RMA-S | KD21-4, 6 |
| none | — | human | H-2K^{b} | RMA-S | D323-4 |
| none | — | human | mCD40 | A20 | RB340 |
| none | — | human | mTLR4 | RAW264.7 | GA467 |
| none | — | human | hTLR4 | THP-1 | 1499 |
| none | — | human | mTLR2 | RAW264.7 | GA518 |
| gp100₂₀₉₋₂₁₇ | HLA-A2 | human | HLA-A2 | T2, hDCs | AV533 |
| gp100₂₀₉₋₂₁₇ | HLA-A2 | human | hTLR4 | hDCs | AV541 |
| idio-peptide | H-2K^{d} | human | H-2K^{b} | RAW264.7 | EY568-39 |
| idio-peptide | H-2K^{d} | human | mTLR4 | RAW264.7 | EY569-31 |

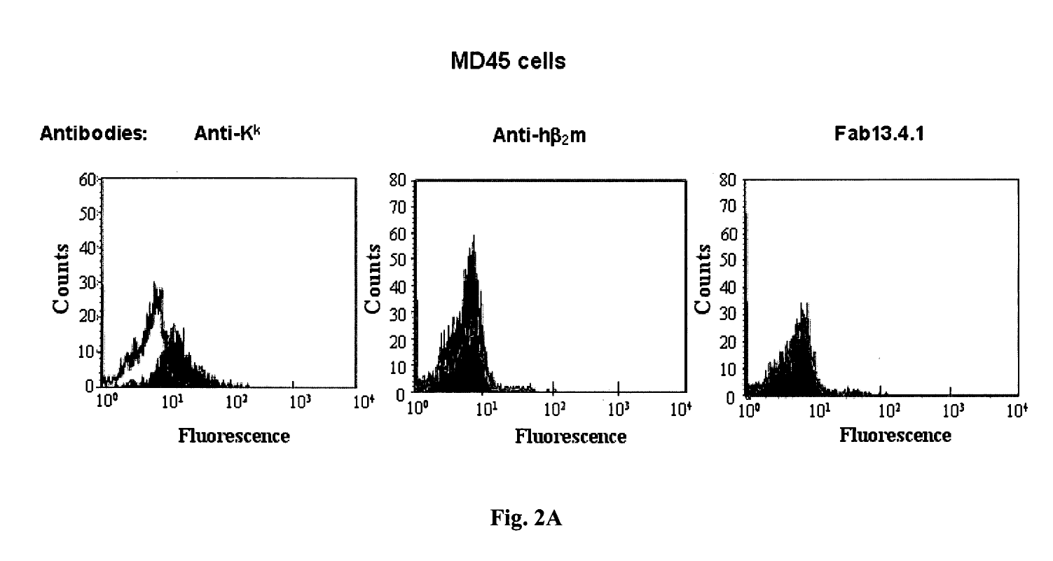

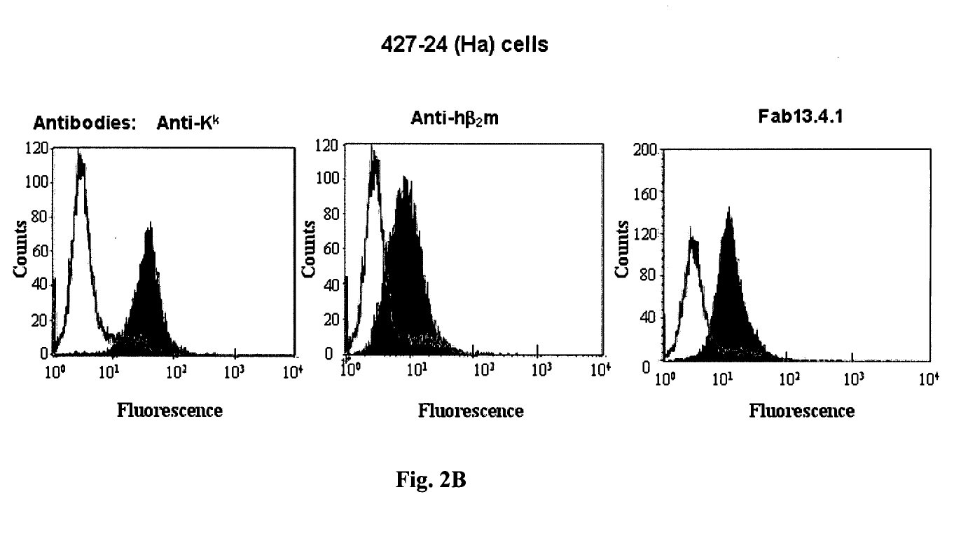

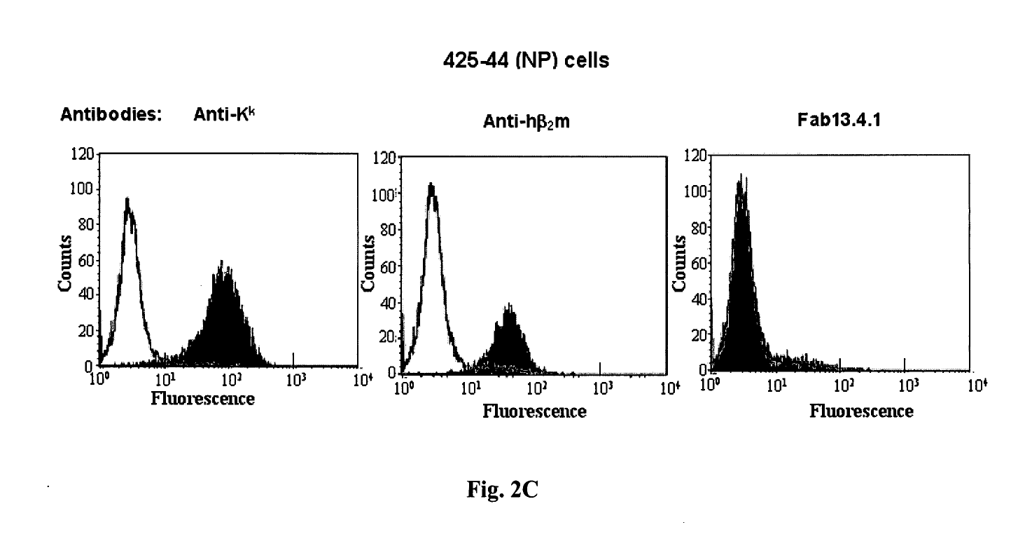

In our previous patent application, WO 01/91698, herein incorporated by reference as if fully disclosed herein, it was aimed to redirect effector T cells against other, harmful T cells, through the CD3 ζ chain portion. In the experimental system described in WO 01/91698, two special mammalian expression cassettes were constructed, which allow the single-step insertion of a stretch coding for an antigenic peptide, so as to create dcβ₂m of either human or mouse origin. The bridging peptide, derived from the human MHC class I molecule HLA-A2, was the extracellular 13-amino acid stretch of SEQ ID NO: 1, which is most proximal to the cell membrane, and the transmembrane and cytoplasmic domains were those of the mouse CD3 ζ chain. The sequence encoding the K^{k}-restricted influenza virus hemagglutinin peptide Ha₂₅₅₋₂₆₂ was cloned into the unique cloning sites in the human β₂m cassette. Plasmid DNA was transfected into the MD45 hybridoma, and one stable transfectant, designated 427-24 (Ha), was further analyzed. Another MD45 transfectant, designated 425-44 (NP), was generated, which similarly expresses the K^{k}-restricted influenza virus nucleoprotein peptide NP₅₀₋₅₇. FACS analysis was performed with the anti-hβ₂m and anti-H-2K^{k} antibodies and with the K^{k}/Ha₂₅₅₋₂₆₂ complex-specific Fab13.4.1. FIG. 2 shows intensive staining of 427-24, but no detectable staining of the control cell 425-44 or of the parental MD45. Quantitative analysis of antigen level on the surface of both transfectants and parental MD45 cells is shown in Table 2 and reveals occupation of 20% of surface H-K^{k} molecules of 427-24 cells by the Ha₂₅₅₋₂₆₂ peptide.

TABLE 2
Quantitative analysis of surface antigens of transfectants
425-44 and 427-24 and parental MD45 cells*

| | Antibody | | |
|---|---|---|---|
| Cell | Anti-H-2K^{k} | Anti-hβ2m | Fab13.4.1 |
| MD45 | 10,909 | 0 | 0 |
| 425-44 | 37,604 | 466,704 | 0 |
| 427-24 | 28,637 | 173,143 | 5,715 |

*Cells were stained with the anti-H-2K^{k} mAb AF3-12.1, the anti-hβ₂m mAb BM-63 and Fab13.4.1, specific to the K^{k}/Ha₂₅₅₋₂₆₂ complex, and analyzed with QIFIKIT (DAKO), using goat anti-mouse IgG (Fab-specific)-FITC conjugated polyclonal antibodies. Mean fluorescence intensities were derived with FACSCalibur software and standard curve was generated from the linear regression of five points at 3,600, 16,000, 53,000, 218,000 and 620,000 mouse IgG molecules per bead, using Excel.

It should be noted that the complex-specific antibody (Fab13.4.1) is a Fab, whereas the anti-H-2K^{k} is an intact IgG. Therefore, the actual occupation of H-2K^{k} molecules on the surface of 427-24 may in fact be higher. Also noteworthy is the 3-fold increase in the total amount of H-2K^{k} in both transfectants 425-44 and 427-24, compared with the parental MD45 cells.

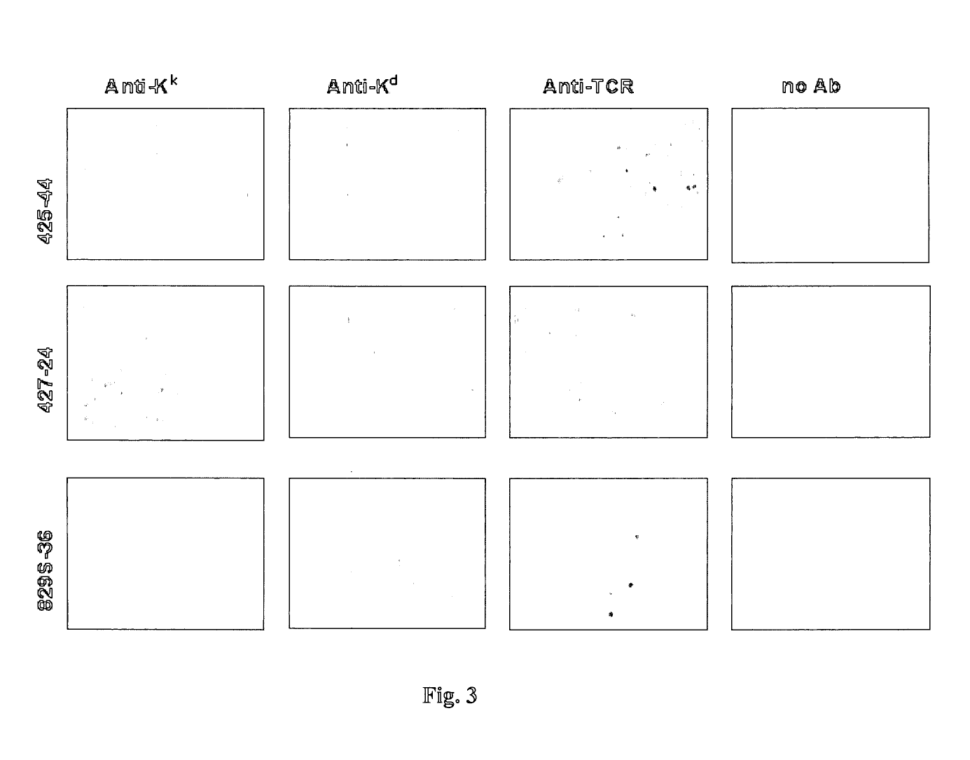

It is conceivable that, on the cell surface, dcβ₂m polypeptides can associate with MHC class I allelic products other than the restricting one. In this scenario, the flexible peptide linker allows the covalently linked antigenic peptide to be situated away from the MHC binding groove, which is occupied by a conventional peptide. In order to test this structural prediction we designed a functional assay, based on the ability of our transfectants to respond to stimulation by Lac-Z production. If this indeed occurs, cells expressing an H-2K^{k} binding peptide will also be activated by an anti-H-2K^{d} mAb, and vice-versa. As shown in FIG. 3, this is really the case. This finding implies to an elevated pool of membranal β₂m, which can become available by lateral diffusion for binding to their cognate MHC class I alleles following dissociation of their original peptide.

### Example 2

### Construction and Expression of dcβ₂m Molecules Harboring Antigenic Peptides of the B16 Mouse Melanoma Model

The APCs for the animal studies are based on the commonly used RMA and RMA-S H-2^{b} cell lines. In the animal experiments, focus is on a mouse melanoma expressing a natural K^{b}-restricted, TAA-derived peptide, and, as a control for peptide specificity, a derivative of the same mouse melanoma is employed presenting another, highly immunogenic K^{b}-restricted peptide, following DNA transfection.

B16 is a spontaneous murine (m) melanoma originating in C57BL/6 mice. B16-F10.9 is a high metastatic line of B16, which shows a low cell surface expression of H-2K^{b}, and K1 is a low metastatic B16 variant, expressing high level of H-2K^{b} following DNA transfection (Porgador et al., 1989). TRP-2 was recently identified as a tumor rejection antigen for the B16 melanoma (Bloom et al., 1997). TRP-2₁₈₁₋₁₈₈, (VYDFFVWL—the peptide of SEQ ID NO: 43, in which the residue S at the amino terminal is absent) is a K^{b}-restricted peptide from TRP-2, and is a major peptide epitope in the induction of tumor-reactive CTLs, which mediate tumor rejection. MO5 is a chicken ovalbumin-transfected variant of the B16 melanoma. It presents the peptide OVA₂₅₇₋₂₆₄ (SIINFEKL—SEQ ID NO: 48), possessing H-2K^{b} anchor residues F at position 5 and L at position 8) in the context of K^{b}.

For further studies, including the B16 model, we replaced both the peptide bridge and the transmembrane and cytoplasmic domains of membranal β₂m with those of the H-2K^{b} molecule. A new XhoI/NotI fragment (see FIG. 1), encoding this polypeptide stretch, was produced, bearing the DNA sequence of

SEQ ID NO: 49:
gag ccc tcg agc tcc act gtc tcc aac atg
gcg acc gtt gct gtt ctg gtt gtc ctt gga gct gca
ata gtc act gga gct gtg gtg gct ttt gtg atg aag
atg aga agg aga aac aca ggt gga aaa gga ggg gac
tat gct ctg gct cca ggc tcc cag acc tct gat ctg
tct ctc cca gat tgt aaa gtg atg gtt cat gac cct
cat tct cta gcg tga.

From the 11^{th} codon (gcg) till the end this sequence encompasses the intact transmembrane and cytoplasmic portion of H-2 K^{b} (positions 658-852 in GenBank accession J00400). The bridge is LRWEPSSSTVSNM (SEQ ID NO: 50), a fusion between the connecting peptide of HLA-A2 (at the carboxyl terminal) and H2-Kb. It is encoded by the sequence ctg aga tgg gag ccC TCG AGc tcc act gtc tcc aac atg, (SEQ ID NO: 51) with an XhoI site incorporated into the sequence.

The sequence of the sense primer comprises 2b protection and an XhoI site followed by the 3′ part of the H-2K^{b} connecting peptide”

(SEQ ID NO: 52)

5′ CCC TCG AGC TCC ACT GTC TCC AAC ATG GCG 3′

The sequence of the reverse primer comprises 3b protection, a NotI site and it corresponds to GenBank accession J00400 positions 858-875:

(SEQ ID NO: 53)

5′ CGC GCGG CCGC AAG TCC ACT CCA GGC AGC 3′

The fragment was produced by RT-PCR performed on mRNA prepared from RMA (H-2^{b}) cells.

The sequences encoding both Trp-2₁₈₁₋₁₈₈ and OVA₂₅₇₋₂₆₄ were cloned as XbaI/BamHI fragments (see FIG. 1) with synthetic oligonucleotides, which were used for PCR amplification of the gene segments encoding mβ₂m leader peptide.

The sequence of the sense primer is:

(SEQ ID NO: 54)

5′ GCG TCT AGA GCT TCA GTC GTC AGC ATG GCT CGC 3′

It comprises 3b protection, an XbaI site and positions 38-61 in GenBank accession X01838, composed of 15 b 5′ non-translated region of mβ₂m leader and the first 3 leader codons, including the ATG.

The sense sequence of the reverse primer for TRP-2₁₈₁₋₁₈₈ is:

(SEQ ID NO: 55)

5′ CTG ACC GGC TTG TAT GCT GTG TAT GAC TTT TTT GTG
TGG CTC GGA GGT GGC GGA TCC GCG 3′

It corresponds to the last 6 codons of the mβ₂m leader, the 8 codons for TRP-218188 (GBA X66349 945-968), the first 5 codons of the linker peptide and 3b protection.

The final (reverse complementary sequence) is:

(SEQ ID NO: 56)

5′ CGC GGA TCC GCC ACC TCC GAG CCA CAC AAA AAA GTC
ATA CAC AGC ATA CAA GCC GGT CAG 3′

The sense sequence of the reverse primer for OVA₂₅₇₋₂₆₄ is:

(SEQ ID NO: 57)

5′ CTG ACC GGC TTG TAT GCT AGT ATA ATC AAC TTT GAA
AAA CTG GGA GGT GGC GGA TCC GCG 3′

It corresponds to the last 6 codons of the mβ₂m leader, the 8 codons for the OVA₂₅₇₋₂₆₄ (GenBank accession J00895, positions 7870-7893), the first 5 codons of the linker peptide and 3b protection.

The final (reverse complementary) sequence is:

(SEQ ID NO: 58)

5′ CGC GGA TCC GCC ACC TCC CAG TTT TTC AAA GTT GAT
TAT ACT AGC ATA CAA GCC GGT CAG 3′

As a BamHI/XhoI fragment encoding the carboxyl terminal of the linker peptide, the full mature hβ₂m and the amino terminal of the bridge we used the same fragment described in WO 01/91698. We created a similar fragment encoding mβ₂m, with the sequence of SEQ ID NO: 59:

gga tcc gga ggt ggt tct ggt gga ggt tcg atc
cag aaa acc cct caa att caa gta tac tca cgc cac
cca ccg gag aat ggg aag ccg aac ata ctg aac tgc
tac gta aca cag ttc cac ccg cct cac att gaa atc
caa atg ctg aag aac ggg aaa aaa att cct aaa gta
gag atg tca gat atg tcc ttc agc aag gac tgg tct
ttc tat atc ctg gct cac act gaa ttc acc ccc act
gag act gat aca tac gcc tgc aga gtt aag cat gac
agt atg gcc gag ccc aag acc gtc tac tgg gat cga
gac atg ctg aga tgg gag ccc tcg agc

From the 11^{th} codon (atc) till the 8^{th} codon before the end (atg) it encompasses positions 113-409 in GenBank accession X01838.

The sequence of the sense primer is:

(SEQ ID NO: 60)

5′ GCG GGA TCC GGA GGT GGT TCT GGT GGA GGT TCG ATC
CAG AAA ACC CCT CAA ATT C 3′

It comprises 3b protection, a BamHI site, a segment encoding the carboxyl terminal of the linker peptide and the first 7 codons of the mature mβ₂m.

The sequence of the reverse primer is:

(SEQ ID NO: 61)

5′ GCG GCT CGA GGG CTC CCA TCT CAG CAT GTC TCG ATC
CCA GTA GAC 3′

It comprises 4b protection, an XhoI site and it corresponds the last 7 codons of mβ₂m and to the amino terminal part of the bridge.

RT-PCR for amplification of mβ₂m sequences was performed on mRNA prepared from MD45 cells. Following verification of DNA sequences, each of the two XbaI/BamHI fragments was cloned into either pCI-Neo or pBJ1-Neo expression vectors, together with the BamHI/Xhol and the XhoI/NotI fragments described herein.

Stable transfectants with the resulting plasmids were generated and are listed in Table 1.

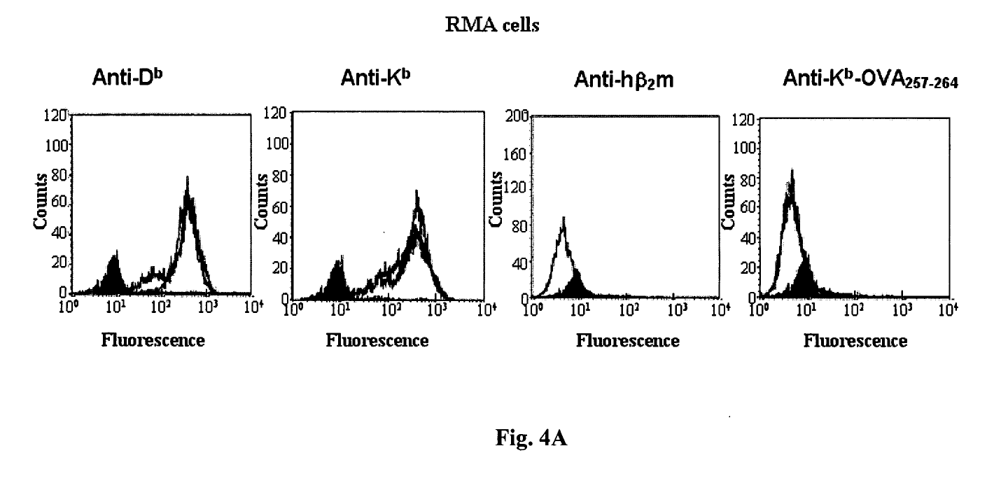

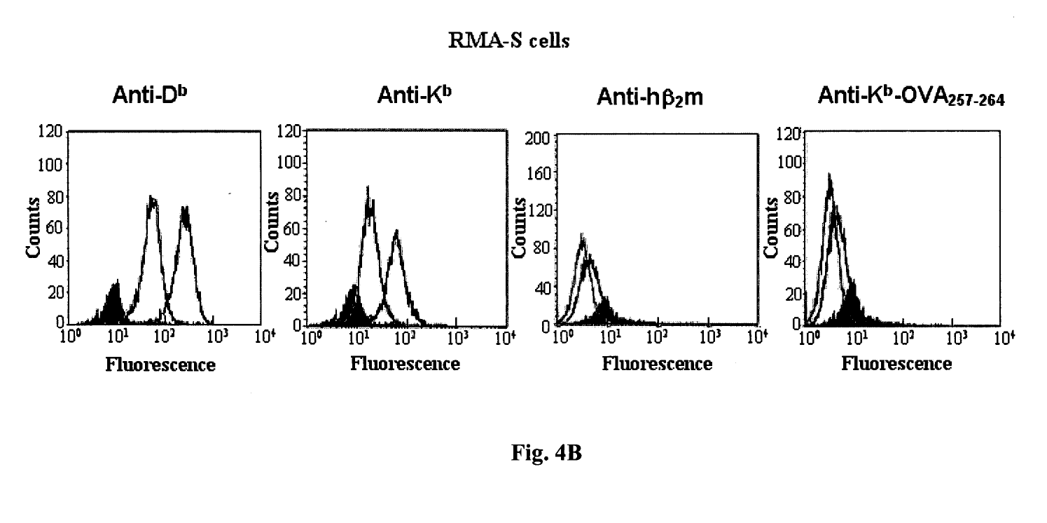

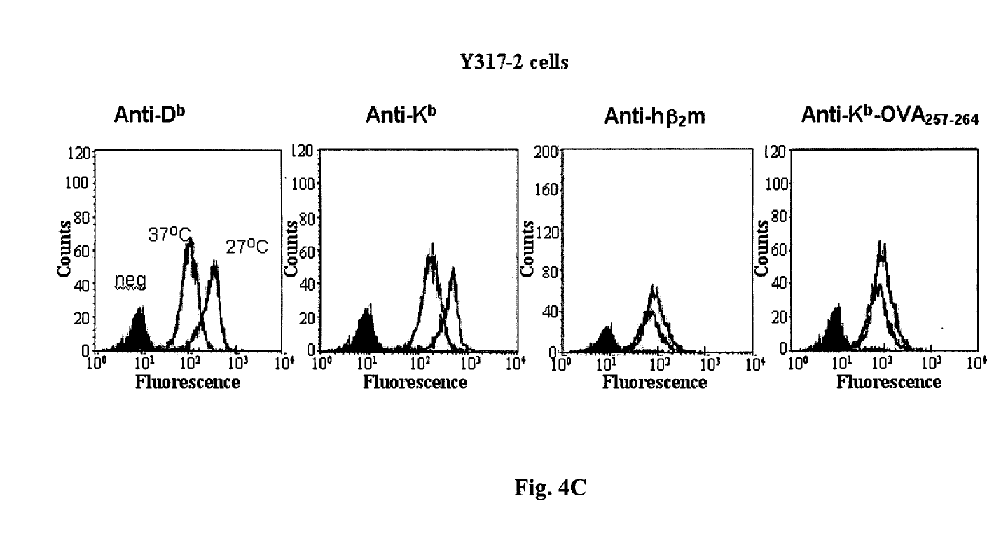

In order to evaluate expression of the new dcβ₂m constructs, we performed the FACS analysis shown in FIG. 4. In this experiment, we compared expression of different MHC class I components on RMA, RMA-S and Y317-2 cells (transfected with OVA₂₅₇₋₂₆₄ fused to membranal hβ₂m), both at 37° C. and 27° C. At this lower temperature MHC class I molecules on the TAP-deficient RMA-S cells are stabilized and their cell surface level increases. It is evident from this analysis that level of both H-2K^{b} and H-2 D^{b} is considerably higher in Y317-2 cells than in their parental RMA-S cells. These results support our previous ones (FIG. 3) in showing that the chimeric polypeptide can associate on the cell surface with allelic products (in this case H-2 Db) other than the one binding the encoded antigenic peptide (Kb). Surface expression of the antigenic K^{b}/OVA₂₅₇₋₂₆₄ complex (as judged by staining with the 25D-1.16 mAb) conclusively indicates that presentation is TAP-independent. Comparison of mean fluorescence intensity (MFI) of expression at 37° C. is presented in Table 3 and reveals 57% H-2K^{b} occupancy in the transfectant.

TABLE 3
Mean fluorescence intensities of clone Y317-2 stained
with an allele-specific and complex-specific mAbs.

Antibody

| Cell | Anti-H-2K^{b} | 25D-1.16 |
|---|---|---|
| Y317-2 | 121.7 | 69.7 |

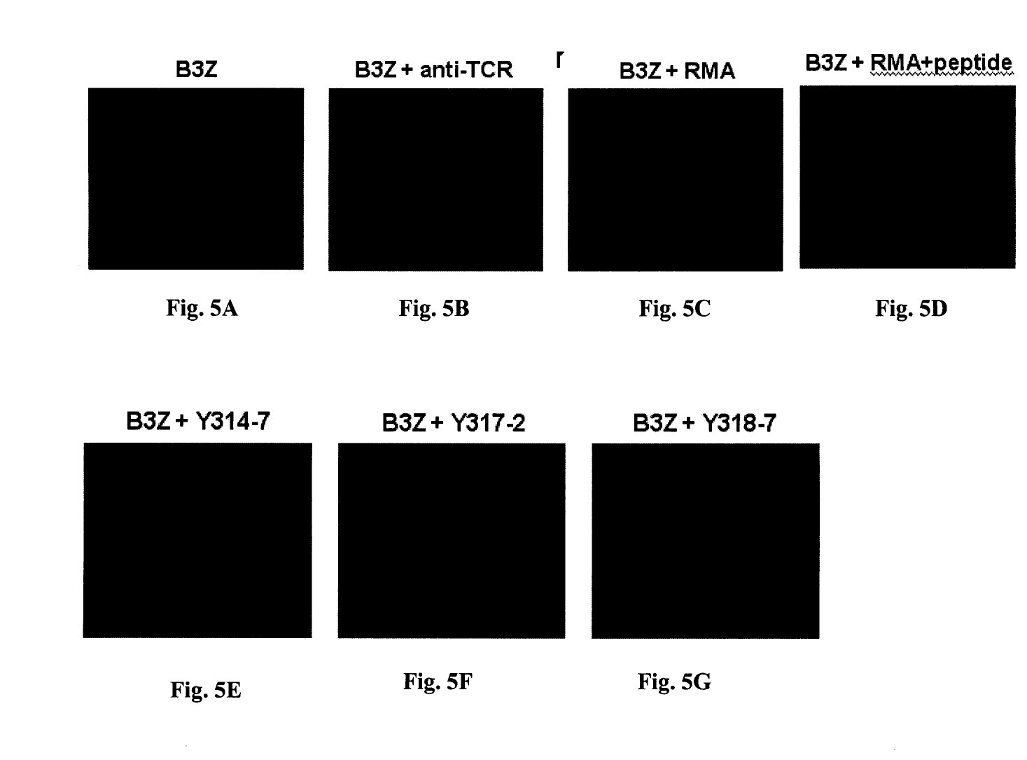

We then went on to confirm that the linker peptide, which joins the carboxyl terminal of the antigenic peptide to the amino terminal of β₂m, does not interfere with T cell recognition. To this end we examined specific activation of B3Z, an H-2K^{b}-restricted, OVA₂₅₇₋₂₆₄-specific CTL hybridoma, by RMA-S clones expressing dcβ₂m with OVA₂₅₇₂₆₄. The results, presented in FIG. 5, show that the level of activation is indistinguishable from that achieved following incubation of parental RMA-S cells with synthetic OVA₂₅₇₋₂₆₄ and rule out major disruption of TCR-ligand interaction in this case.

### Example 3

### Evaluating Contribution of Membrane Anchorage of β₂m to MHC Class I Stability

In the experimental system described in WO 01/91698, a plasmid was assembled, designated 21-2, which encodes a membranal hβ₂m, linked to the transmembrane and cytoplasmic region of mouse CD3 ζ chain. Another plasmid, 323-3 was assembled, in which the CD3 ζ portion was replaced with those of H-2Kb. This was done as follows:

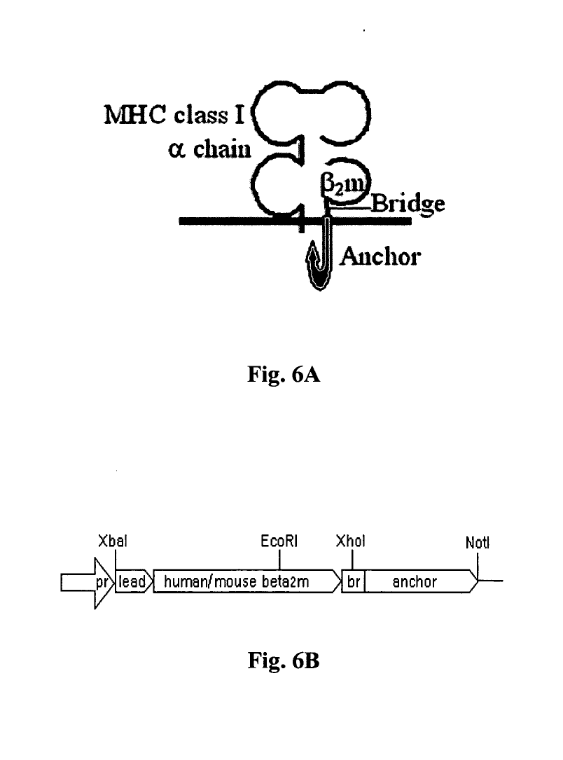

Scheme of genetic constructs encoding these single chimeric β₂m (scβ₂m) derivatives and of their expected polypeptide products associated with an MHC class I heavy chain are illustrated in FIG. 6.

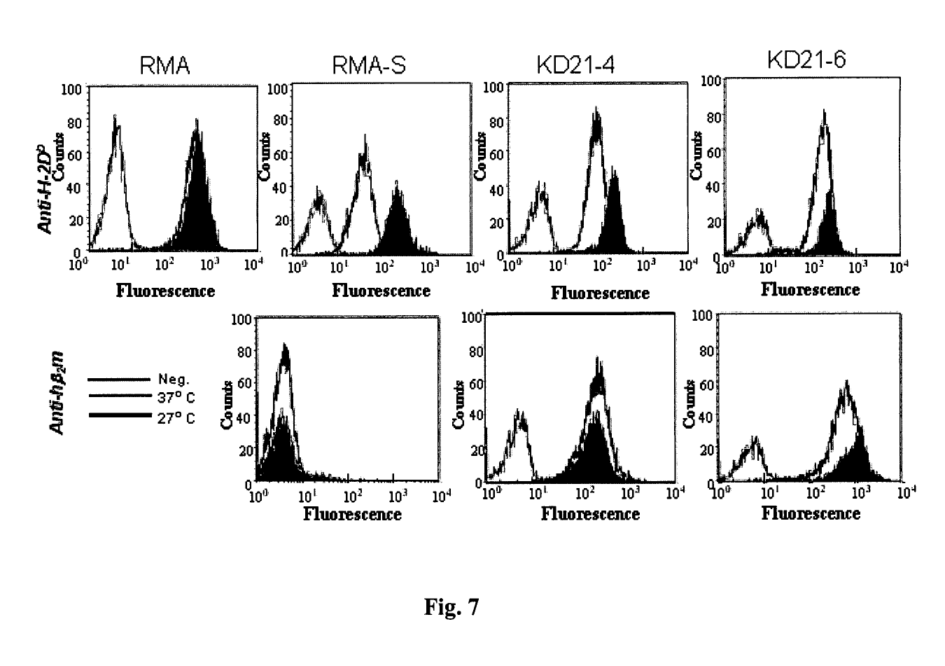

Plasmid 21-2 was introduced into RMA-S cells. Following FACS analysis of G418-resistant transfectants with the anti-hβ₂m antibody, two clones, designated KD21-4 and KD21-6, were chosen, the latter expressing higher level of membranal β₂m. These two clones were analyzed for the ability of the scβ₂m product to stabilize the MHC class I molecule H-2D at 37° C. Results of a typical experiment are presented in FIG. 7. It is clear from these results that H-2D^{b} level is elevated at 37° C. compared with the parental RMA-S cells, and that this elevation correlates with expression level of hβ₂m. In fact, for KD21-6, the level of surface H-2 Db is comparable to that of the wild-type RMA cells.

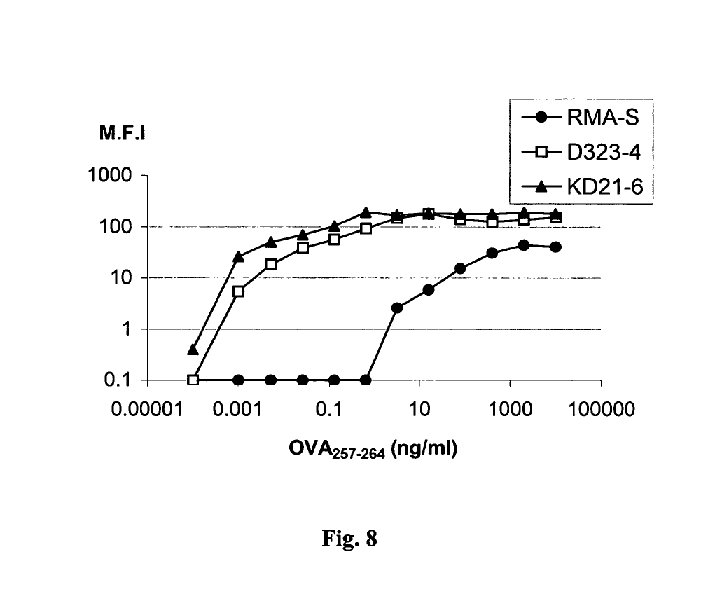

Plasmid 323-3 was similarly introduced to RMA-S cells and a stable transfectant, designated D323-4, which expresses high level of hβ₂m, was selected. In the next experiment we evaluated the ability of both KD21-6 and D323-4 transfectants to bind exogenously added synthetic OVA₂₅₇₋₂₆₄ peptide through H-2K^{b}, in comparison with parental RMA-S cells, exploiting the complex-specific 25D-1.16 mAb. This experiment was repeated 6 times, producing essentially identical results. Results of one of these experiments are shown in FIG. 8. They demonstrate approximately 3 logs enhancement of the ability to bind exogenous peptide, while maximal level of binding increases only 3-4-fold compared with RMA-S cells. These findings imply that expression of the scβ₂m products results in a vast enhancement in the functional affinity of the antigenic peptide to the MHC class I molecule. It should be noted that the nature of the β₂m anchor (CD3 ζ in KD21-6 or H-2K^{b} in D323-4) has little influence on the magnitude of this striking phenomenon.

### Example 4

### In-Vivo Assessment of dcβ₂m-based APCs

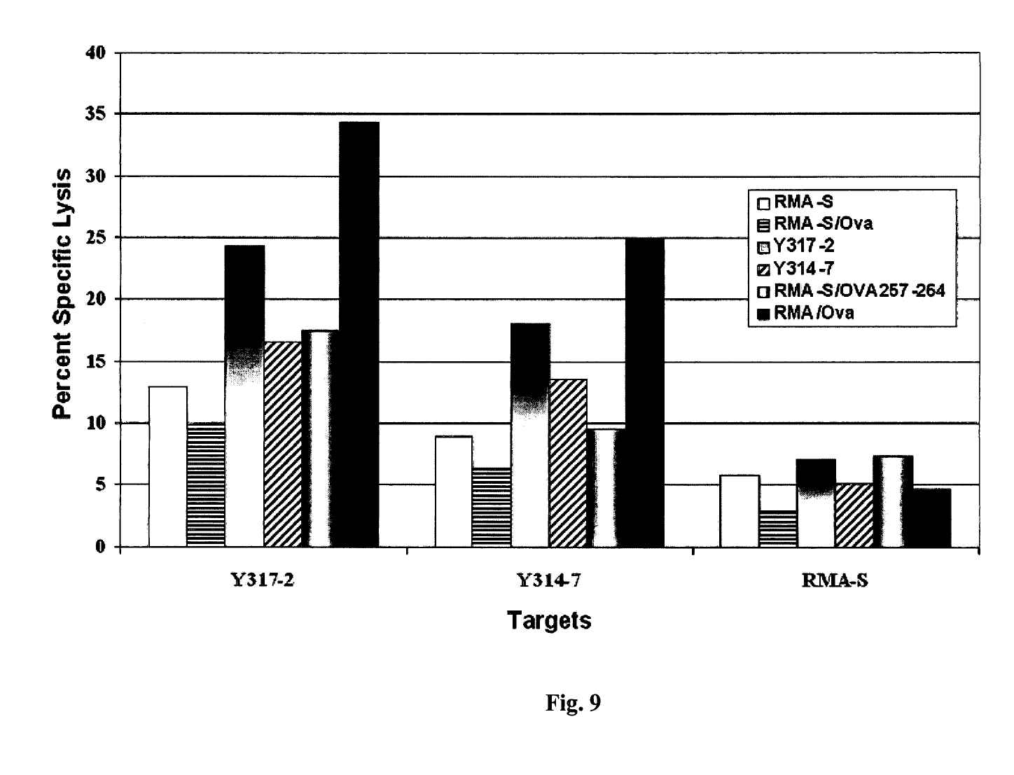

For in vivo evaluation of the capacity of dcβ₂m-based APCs to induce a specific CTL response, the RMA-S transfectants Y317-2 and Y314-7, expressing OVA₂₅₇₋₂₆₄ linked to hβ₂m or mβ₂m, respectively, were compared with cells exogenously loaded by peptides. In a preliminary experiment, C57BL/6 (B6) mice were immunized with the indicated cells. CTLs prepared from immunized mice were used in a cell cytotoxicity assay, in which transfectants were evaluated as target cells at various effector/target ratios. Results are depicted in FIG. 9 and indicate that both Y317-2 and Y314-7 cells can serve as immunogens and as target cells for CTLs. These finding reinforce our previous conclusion that dcβ₂m is an efficient vehicle for presentation of pre-selected antigenic peptides and that the linker peptide does not interfere with T cell recognition.

### Example 5

### Assembly and Preliminary Evaluation of β₂m Fused to CD40 Trans-Membrane and Cytoplasmic Region

DC licensing requires engagement of the CD40L on the CD4 T cell with CD40 on the DC and is a mandatory step in the elicitation of many CTL responses. We reasoned that supplementing β₂m with the intracellular portion of CD40 might trigger CD40 signaling upon encounter of DCs expressing these new dcβ₂m constructs with specific CTLs, circumventing CD4 T cell help. In other words, the CD40 signaling moiety can serve as an adjuvant in membranal β₂m-based vaccines. To test this idea we assembled a new scβ₂m expression plasmid (encoding hβ₂m, according to the general scheme illustrated in FIG. 6), in which the encoded anchor comprises CD40 transmembrane and cytoplasmic portion. This was done as follows:

The bridge is LRWEPSSSTVSNM (SEQ ID NO:50), a fusion between the connecting peptide of H-Kb with that of HLA-A2, as in Example 2. The gene segment encoding mouse CD40 transmembrane and cytoplasmic region encompasses positions 588-878 in GenBank accession M83312 and its DNA sequence (SEQ ID NO: 62) is:

gcc ctg ctg gtc att cct gtc gtg atg ggc atc
ctc atc acc att ttc ggg gtg ttt ctc tat atc aaa
aag gtg gtc aag aaa cca aag gat aat gag atg tta
ccc cct gcg gct cga cgg caa gat ccc cag gag atg
gaa gat tat ccc ggt cat aac acc gct gct cca gtg
cag gag aca ctg cac ggg tgt cag cct gtc aca cag
gag gat ggt aaa gag agt cgc atc tca gtg cag gag
cgg cag gtg aca gac agc ata gcc ttg agg ccc ctg
gtc tga.

The sequence of the sense primer (SEQ ID NO: 63) is:

5′ CCC TCG AGC TCC ACT GTC TCC AAC ATG GCC CTG
CTG GTC ATT CCT G 3′.

It comprises 2b protection, an XhoI site followed by the 3′ part of the segment encoding the bridge and the first 19b encoding the CD40 portion.

The sequence of the reverse primer (SEQ ID NO: 64) is:

5′ CGC GCG GCC GCG GTC AGC AAG CAG CCA TC 3′

It corresponds to a stretch downstream the CD40 stop codon (positions 901-918 in GenBank Accession M83312) and contains NotI and 3b protection.

Messenger RNA was prepared from the murine B cell lymphoma A20, known to express CD40, and RT-PCR was performed with the two primers. The 369 bp product was cloned into pGEMT and DNA sequence was confirmed. The XhoI-NotI fragment was excised and inserted into the expression vector pBJ1-Neo cut with XbaI and NotI, together with the XbaI-XhoI fragment from plasmid 21-2, encoding hβ₂m with its leader peptide and the amino terminal of the bridge.

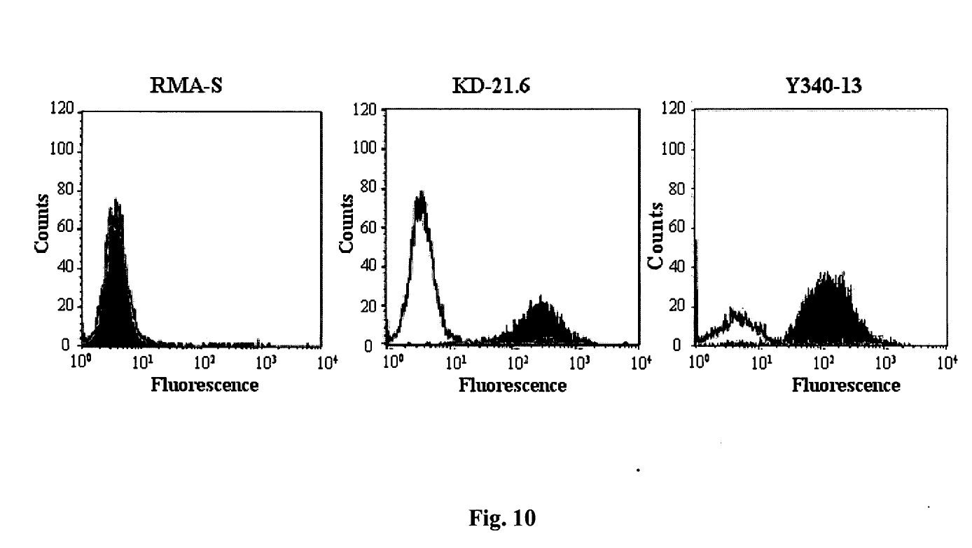

In order to assess function of the CD40 domains, we took advantage of the finding that CD40 can activate the nuclear factor of activated T cells (NFAT) (Choi et al., 1994). Plasmid DNA was introduced into B3Z cells (capable of high LacZ expression following stimulation through the NFAT-LacZ reporter gene) and resulting clones were screened for hβ₂m expression. FACS analysis, shown in FIG. 10, reveals high expression of hβ₂m in one of the transfectants (Y340-13) but none in the parental B3Z cells.

### Materials and Methods for Example 6

(i) Vectors and expression plasmids. Chimeric β₂m genes were cloned into the mammalian expression vectors pBJ1-Neo or pCI-Neo (Promega, Madison, Wis.). An XbaI/BamHI stretch coding for mouse β₂m (mβ₂m) leader peptide, the H-2K^{b}-binding antigenic peptide and the N-terminal part of the linker peptide was constructed with the forward primer 5′GCG TCT AGA GCT TCA GTC GTC AGC ATG GCT CGC 3′ (SEQ ID NO: 54) and the reverse primer 5′CGC GGA TCC GCC ACC TCC CAG TTT TTC AAA GTT GAT TAT ACT AGC ATA CAA GCC GGT CAG 3′ (SEQ ID NO: 58) for OVA₂₅₇₋₂₆₄ (SIINFEKL, SEQ ID NO: 48), 5′CGC GGA TCC GCC ACC TCC GAG CCA CAC AAA AAA GTC ATA CAC AGC ATA CAA GCC GGT CAG 3′ (SEQ ID NO: 56) for TRP-2₁₈₁₋₁₈₈ (VYDFFVWL, SEQ ID NO: 43), or 5′CGC GGA TCC GCC ACC TCC CGG CTG GGC TGT GTT ACA CTC AAA AGC ATA CAA GCC GGT CAG 3′ (SEQ ID NO: 80) for MUTI (FEQNTAQP, SEQ ID NO: 81). Cloning of a BamHI/XhoI fragment encoding mature human β₂m (hβ₂m) with the C-terminal part of the linker peptide and the N-terminal part of the bridge was described. An analogous stretch containing the mature mβ₂m was cloned by RT-PCR using the forward primer 5′ GGC GGA TCC GGA GGT GGT TCT GGT GGA GGT TCG ATC CAG AAA ACC CCT CAA 3′ (SEQ ID NO: 82) and the reverse primer 5′ AAG ACC GTC TAC TGG GAT CGA GAC ATG CTG AGA TGG GAG CCC 3′ (SEQ ID NO: 83). The template for mβ₂m gene segments was mRNA from the MD45 T cell hybridoma (H-2^{k/d}) and the gene product encodes Asp at the polymorphic position 85. The production of an XhoI/NotI fragment encoding the peptide bridge and the transmembrane and cytoplasmic portion of H-2K^{b} was described elsewhere. All PCR products were subcloned and their DNA sequence verified. The complete genes were assembled via a single step insertion of the three corresponding fragments into the multiple cloning site of either vector.

(ii) Mice and cell lines. Eight-12-week old C57BL/6 (B6) mice were purchased from Jackson Laboratory (Bar Harbor, Me.) and bred at the Weizmann Institute of Science (WIS, Rehovot, Israel) facilities. Animals were maintained and treated according to the WIS animal facility and National Institutes of Health (NIH) guidelines.

RMA, RMA, RMA-S:OVA, MO5 cells—see “Materials and Methods for Examples 1-5” herein (i, Cells). MO5 cells were maintained in DMEM supplemented with 10% heat-inactivated FCS, 2 mM L-glutamine, 1% sodium pyruvate, 1% non-essential amino acids, combined antibiotics and 500 μg/ml G-418 (Life Technologies, Gaithersburg, Md.).

(iii) Peptides. OVA₂₅₇₋₂₆₄ and TRP-2₁₈₁₋₁₈₈ were synthesized by Dr. M. Fridkin, W.I.S.

(iv) DNA transfection. RMA-S (0.8 ml) at 4×10⁶ cells/ml were mixed in 4 mm sterile electroporation cuvette (ECU-104, EquiBio, Ashford, UK) with 20 μg linearized plasmid DNA. Transfection was performed with an Easyject Plus electroporation unit (EquiBio) at 250V, 750 μF. Cells were resuspended in fresh medium and cultured for 24-48 hours in 96-well plates prior to addition of G418 to a final concentration of 1 mg/ml. Resistant clones were expanded in 24-well plates and screened by flow cytometry for expression of hβ₂m or increase in expression of surface H-2K^{b}.

(v) Tumor immunotherapy. Ten mice in each experimental group were inoculated s.c. in the upper back with 1×10⁵ MO5 cells/mouse. Local tumor diameter was measured with calipers. Starting 8 days later, when the tumor reached 3-4 mm in diameter, mice were immunized i.p. four times at 7-day intervals with 2×10⁶ irradiated transfectants or control cells pre-loaded with peptide at 50 μg/ml. Tumor diameter and survival were recorded.

(vi) Statistical analysis. Statistical differences in tumor sizes between groups of mice was determined by one-way ANOVA. Significance of survival plots was done with Kaplan-Meier survival platform. For both analyses we used the JMP statistics software (SAS Institute, Cary, N.C.).

### Results of Example 6. Immunotherapy of Tumors.

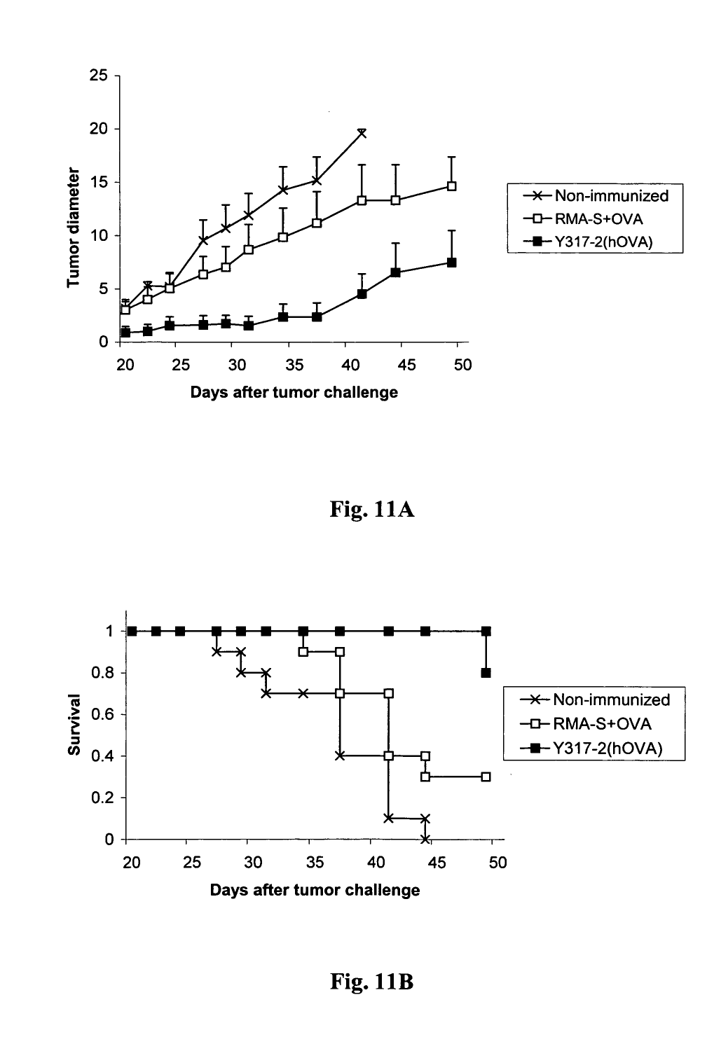

To evaluate immunotherapy of melanoma, we performed an experiment of tumor growth inhibition. B6 mice were challenged with 1×10⁵ MO5 cells each. Starting eight days later, mice were subjected to an immunization regimen with either irradiated Y317-2(hOVA), parental RMA-S cells pulsed with OVA₂₅₇₋₂₆₄, or with PBS only as control. As evident from FIG. 11A, tumor growth was significantly delayed in mice vaccinated with Y317-2(hOVA) compared to the peptide-loaded cells (p<0.0001). This therapeutic effect was also evident from the survival graph (FIG. 11B). Of 10 mice vaccinated with Y317-2(hOVA), 8 were still alive 7 weeks after tumor challenge, as opposed to only 3/10 of mice vaccinated with RMA-S cells loaded with the peptide (p<0.0001) and 0/10 of non-immunized mice. In contrast, immunization with Y318-10(hTRP) and TRP-2₁₈₁₋₁₈₈-loaded RMA-S cells under the same experimental conditions failed to yield any significant MO5 suppression effect (data not shown).

### Materials and Methods for Examples 7-11

(i) Cells. RAW264.7 is a mouse macrophage cell line. THP-1 is a human monocytic cell line. XS52 is a mouse DC line established from the epidermis of newborn BALB/c (H-2 d) mice and is a gift from Dr. A. Takashima (University of Texas). CHIB2 is a hybrid of the H-2 K^{d}-restricted, insulin B chain peptide-(B15-23)-specific G9C8 T cell clone with the BW5147 thymoma expressing CD8 and the NFAT-lacZ reporter gene {BWZ.36 CD8α}. In our study we incorporated a higher affinity derivative of B15-23 {Val instead of Gly at position 9, or G9V}. Human DCs were derived and propagated ex-vivo as follows: Peripheral blood mononuclear cells (PBMCS) were isolated from a healthy donor by a ficoll gradient following cytopheresis. To obtain immature DCs, adherent cells were cultured for 6 days in complete RPMI medium with 10% AB serum, supplemented with 1000 u/ml GM-CSF and 750 U/ml IL-4. To generate mature DCs, day 5 cells were grown for additional 24 h in the presence of a maturation cocktail containing TNFα (1000 U/ml), IL-6 (10 ng/ml), IL1-β (5 ng/ml) and PGE2 (0.35 μg/ml).

(ii) Antibodies. BM-63 is an anti-human β₂m mAb (Sigma). FITC-conjugated anti CD86 is from DAKO.

(iii) DNA transfection. See “Materials and Methods for Examples 1-5” herein (iii, DNA transfection). In this study a different electroporation device (Bio-Rad Gene Pulser II Xcell System) was used.

(iv) RNA preparation and transfection of DCs and APC lones. Genes of interest were cloned into the multiple cloning site of a special vector designed for in-vitro production of mRNA {pEGM4Z/GFP/A64}, following removal of the GFP gene. Messenger RNA was prepared from cloned templates using the T7 mMessage mMachine Kit (Ambion) following plasmid linearization with SpeI. Immature and mature DCs were harvested and washed two times in cold PBS, then resuspended in cold Opti-MEM at 2-3×10⁶ cells/100 μl and were mixed in 2 mm gap cuvette along with 5-10 μg of in-vitro transcribed mRNA. The mixture was then subjected to 300 V for 5001s in a square wave electroporator (BTX-ECM 830, San Diego, Calif.). APC lines (RAW and XS52) were transfected with the same amount of in-vitro transcribed RNA, using 2 mm cuvettes and Bio-Rad Gene Pulser II Xcell System.

(v) FACS analysis. See “Materials and Methods for Examples 1-5” herein (iv, FACS analysis).

(vi) Cell stimulation assay. Stimulator cells and responders at 5×10⁵ cells/ml each were incubated overnight at 37° C. in 96-well plates in triplicates, in a total volume of 100 μl. Medium was removed and cells were resuspended in 100 μl of lysis buffer {9 mM MgCl₂, 0.125% NP40, 0.3 mM chlorophenol red β-D galactopyranoside (CPRG) in PBS}. Following 1-12 hours incubation in dark, post-lysis optical density (O.D.) was monitored with an ELISA reader at 570 nm with 630 nm as reference.

(vii) Semi-quantitative RT-PCR analysis of gene expression. Total RNA from 5×10⁶ cells was purified using the TRI reagent (Sigma). 1 ug of total RNA was used for cDNA preparation using AMV RT enzyme (Promega) and an oligo-dT primer. PCR was performed using specific primers for the analyzed gene.

### Example 7

### Incorporating Mouse and Human TLR4 and Mouse TLR2 Portions into dcβ₂m

The transmembrane (tm) and cytoplasmic (cyt) portion of both mouse and human TLR4 were engrafted as the ‘anchor’ segment in the dcβ₂m modality, depicted in FIG. 12 at the gene level as the XhoI-NotI fragment. The respective DNA stretches were amplified by RT-PCR performed on RNA from RAW264.7 cells using specific oligonucleotide primers, and were provided with suitable restriction sites for cloning. In all experiments described in this chapter we used human β₂m (hβ₂m).

(i) Mouse TLR4. For cloning mouse TLR4 (mTLR4), we based our primer design on data available in Genbank accession (GBA) AF110133. The forward primer spans positions 1891-1914 in the sequence. and is preceded by a SalI restriction site (underlined, rather than XhoI, which occurs within the cloned stretch):

(SEQ ID NO: 84)

5mTLR4:
5′ CCG TCG ACC ACC TGT TAT ATG TAC AAG ACA ATC 3′

(All primers start with a 2-3 C/G nucleotide stretch for protecting the ends of the resulting PCR products.) The reverse primer spans positions 2543-2560, which are located downstream to the mTLR4 stop codon (2527-2529) and harbors a NotI site:

(SEQ ID NO: 85)

3mTLR4:
5′ CGC GCG GCC GCA CTG GGT TTA GGC CCC AG 3′

These primers were used to amplify a gene fragment encoding mTLR4 tm+cyt portion, following by subcloning and DNA sequence verification. It was then cloned into a mammalian expression vector {pBJ1-Neo or pCI-Neo (Promega)} as a SalI-NotI piece in the context of either peptide-less membranal hP₂m or in conjunction with the same platform encoding different antigenic peptides (FIG. 12).

(ii) Human TLR4. RT-PCR amplification of the corresponding domain from human TLR4 (hTLR4) from RNA of the human monocytic THP-1 cell line and subsequent cloning were carried out similarly to the procedure described for mTLR4. Primer design was based on GBA NM138554. The forward primer spans positions 2043-2063 in the sequence and is preceded by a XhoI restriction site:

(SEQ ID NO: 86)

5hTLR4;
5′ CCC TCG AGC ACC TGT CAG ATG AAT AAG ACC 3′

The reverse primer spans positions 2704-2726, which are located downstream to the mTLR4 stop codon (2685-2687) and harbors a NotI site:

(SEQ ID NO: 87)

3hTLR4:
5′ CGC GCG GCC GCT GGG CAA GAA ATG CCT CAG GAG GT
3′

(iii) Mouse TLR2. RT-PCR amplification of the corresponding domain from mouse TLR2 (mTLR2) from RNA of the RAW264.7 cell line and subsequent cloning were carried out similarly to the procedure described for mTLR4. Primer design was based on GBA NM011905. The forward primer spans positions 2276-2293 in the sequence and is preceded by a XhoI restriction site:

(SEQ ID NO: 88)

| | |
|---|---|
| 5mTLR2; | 5′ CCC TCG AGC GCA CTG GTG TCT GGA GTC 3′ |

The reverse primer spans positions 2875-2892, which are located downstream to the mTLR2 stop codon (2864-2866) and harbors a NotI site:

(SEQ ID NO: 89)

3mTLR2:
5′ CGC GCG GCC GCA GGA AGT CAG GAA CTG GG 3′

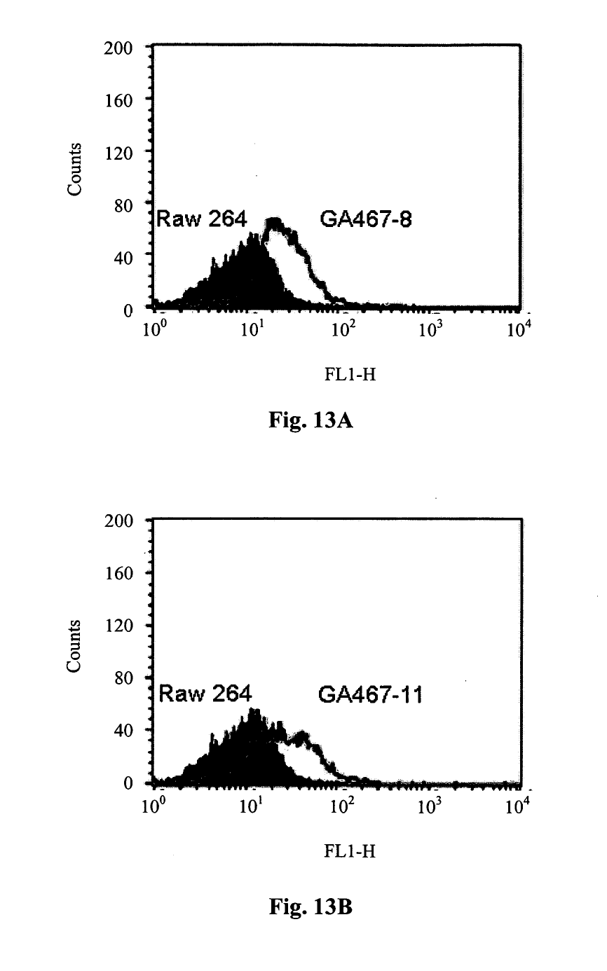

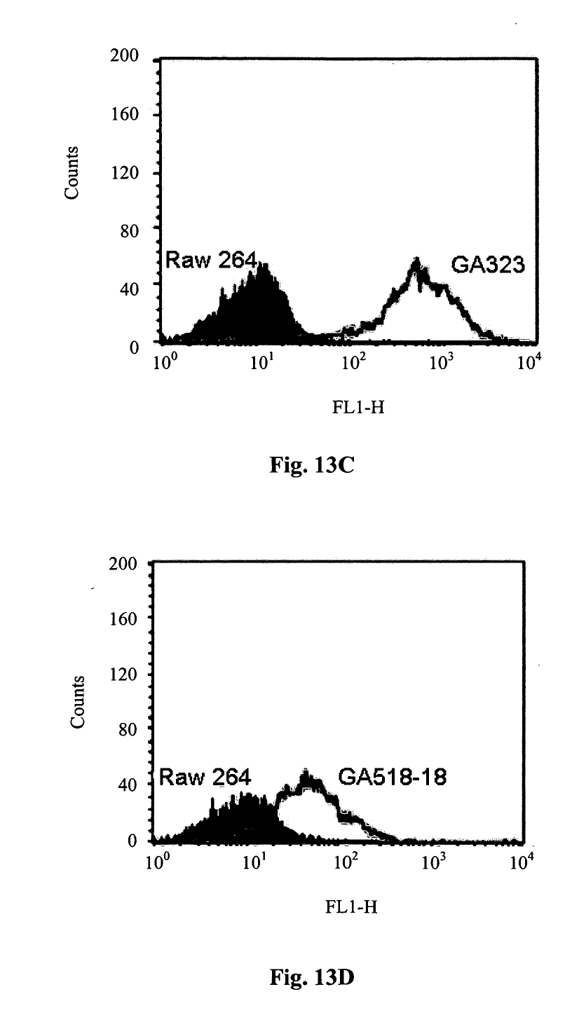

As a control for some experiments we used plasmid 323, which codes for hβ₂m fused with an H-2K^{b}-derived anchor (Berko et al., 2005). The final expression plasmids were used to generate stable transfectants of THP-1 (hTLR4, clones 1499-3 and 4) and RAW264.7 (mTLR4: GA467-8 and 11, mTLR2: GA518-18 and 323: GA323) cells (FIG. 13).

### Example 8

### Peptide-Less β₂m-TLR4 Fusion Constructs Confer a Constitutively Activated Phenotype on Transfected Human and Mouse Monocytic Cell Lines

We first evaluated the ability of the membrane-anchored hβ₂m construct fused with the activation domain of TLR4 to induce constitutive production of Th1-polarizing pro-inflammatory cytokines in transfected monocytic cell lines, as was originally shown by Medzhitov et al. (1997).

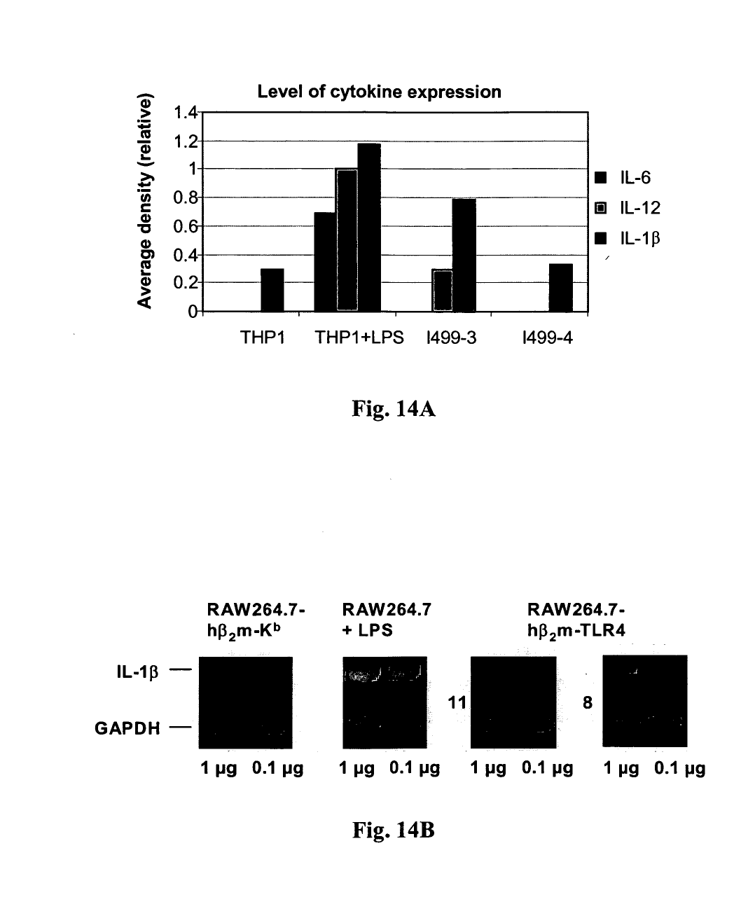

Preliminary analysis of cytokine expression was performed by semi-quantitative RT-PCR using specific pairs of oligonucleotide primers, while expression of GAPDH served as reference. FIG. 14A shows results obtained with a positive (1499-3) and a negative (1499-4) THP-1 transfectant identified among the few clones analyzed so far and of parental THP-1 cells. As a positive control we stimulated THP-1 cells with the TLR4 ligand LPS. The expression pattern indeed reveals substantial elevation in the basal level of IL-12 and IL-1β and indicates that the gene product confers a constitutively activated state. Similar results were obtained with RAW264.7 cells (FIG. 14B). In this setting we used as a negative control RAW264.7 cells stably expressing the hβ₂m-H-2K^{b} construct and LPS-stimulated cells as the positive control. Elevation in the basal level of IL-11 is evident in the two clones shown (No. 11 and 8).

### Example 9

### Full dcβ₂m-TLR4 Fusion Constructs are Constitutively Functional in Transfected RAW264.7 Cells

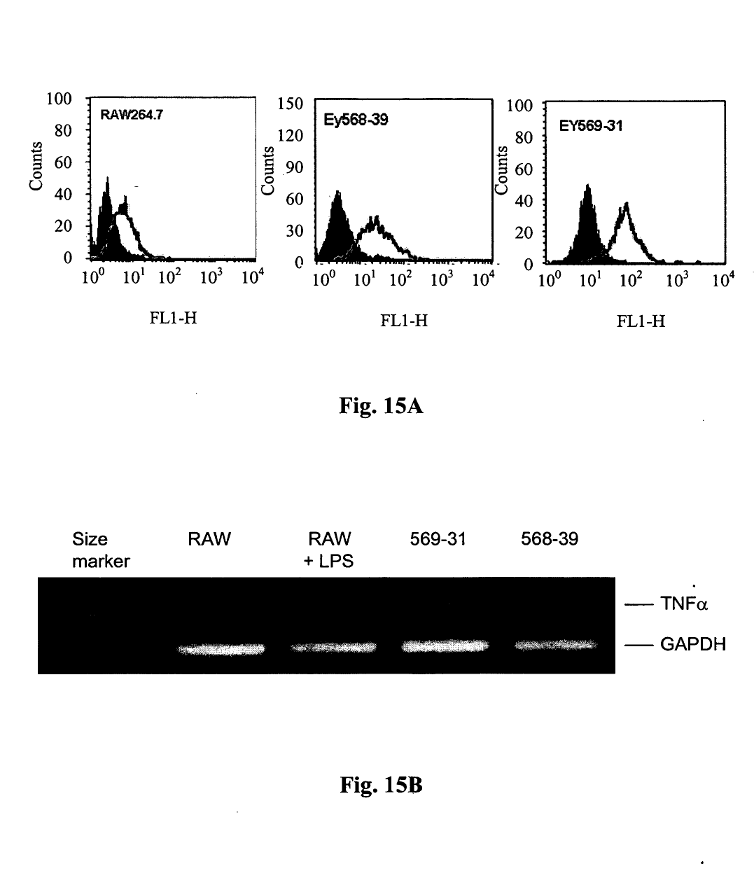

In this experimental system we have genetically engrafted an H-2 K^{d}-binding idiotypic peptide expressed by a mouse myeloma onto either hp₂m-mTLR4 or hβ₂m-H-2K^{b} (an ‘inert’ anchor as a control) and generated stable transfectants of the RAW264.7 cell line (H-2^{d}). These were first screened by flow cytometry for surface expression of hβ₂m against parental RAW264.7 cells. Two clones were selected for further analysis (see FIG. 15A): Ey569-31 (with the TLR4 anchor) and Ey568-39 (with the H-2K^{b} anchor). The weak staining of parental cells is attributable to the expression of FcγR. We then performed a preliminary RT-PCR analysis for the constitutive expression of TNFα by Ey569-31. As a positive control we stimulated parental RAW264.7 cells with LPS. Indeed, clone Ey569-31 showed a clear band of the expected size, whereas nonstimulated RAW264.7 cells and clone Ey568-39 showed only a very faint band as expected, representing basal expression of TNFα (FIG. 15B).

### Example 10

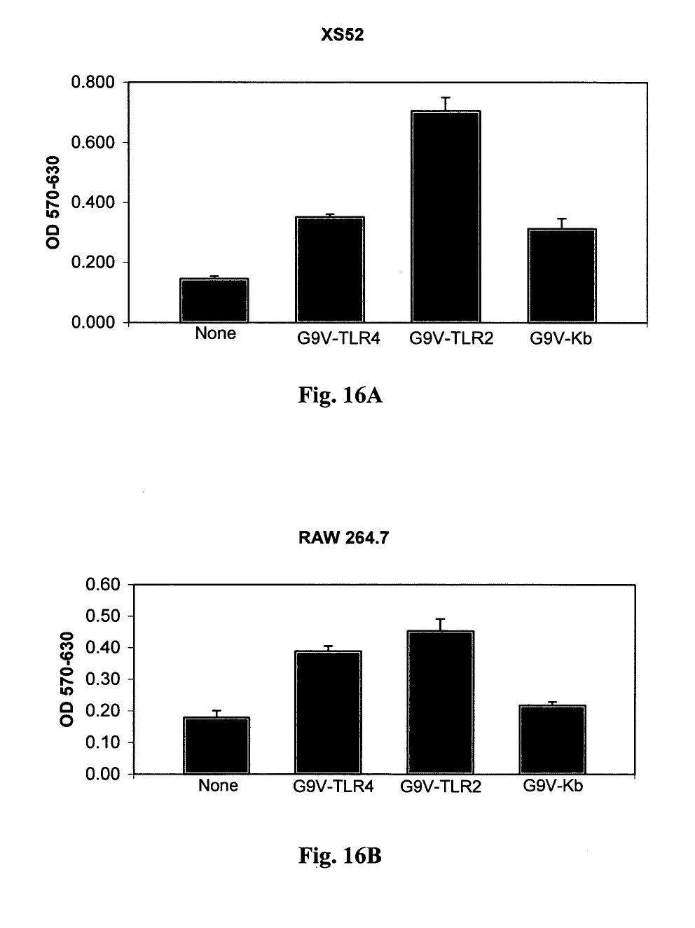

An antigenic peptide linked to a mTLR4- and mTLR2-bearing dcβ₂m construct stimulates a mouse T cell hybridoma. To demonstrate functional pairing of a TLR4-based construct with MHC-I heavy chain at the cell surface, we tested whether an antigenic peptide genetically linked to hβ₂m-mTLR4 or hP₂m-mTLR2 can stimulate a peptide-specific, MHC-I restricted T cell hybridoma. To this end we linked the mouse insulin B chain heteroclitic peptide G9V as a model H-2 K^{d}-binding peptide to the N-terminus of hβ₂m-mTLR4 and hβ₂m-mTLR2 scaffolds and used CHIB2 as the responder T cell. We included the same peptide fused with hβ₂m-H-2 K^{b} as a positive control. The three genetic constructs were used to as templates for in-vitro transcription and the resulting RNA was used to for transient transfection of the immature mouse DC line XS52 (H-2^{d}). FIG. 16 shows stimulation of the CHIB2 hybridoma by XS52 or RAW264.7 cells transfected with the three dcβ₂m-encoding mRNA, but not by parental XS52 or RAW264.7 cells treated similarly.

### Example 11

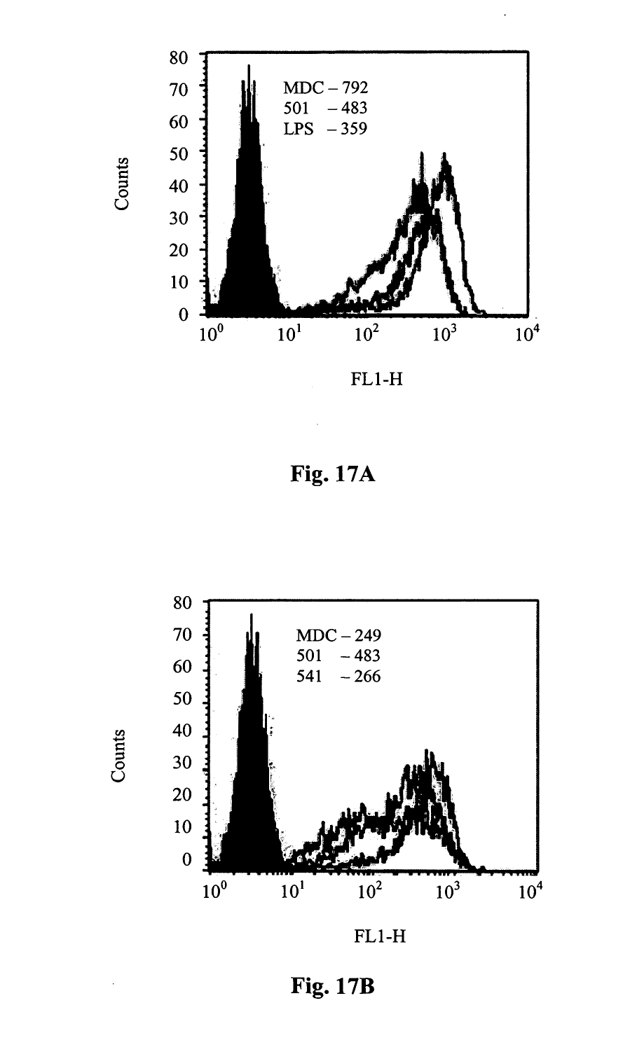

Ex-vivo propagated human DCs are activated following transfection with RNA encoding full dcβ₂m-TLR4 fusion constructs. The experimental setting we describe in this example is pertinent to ex-vivo immunization protocols we are developing for the immunotherapy of human melanoma. We have genetically engrafted an HLA-A2 binding peptide from the human melanocyte differentiation antigen gp100 {gp100₂₀₉₋₂₁₇, ITDQVPFSV (Kawakami et al., 1995) onto hβ₂m, fused either with hTLR4 or an HLA-A2-derived inert anchor (see FIG. 12). Our data from preliminary experiments (not shown) reveal that the gp100₂₀₉₋₂₁₇-hβ₂m-A2 dcβ₂M polypeptide functionally pairs with HLA-A2 at the surface of transfected human APCs. We then went on to compare the ability of the gp100₂₀₉₋₂₁₇-hβ₂m-TLR4 construct to that of the gp100₂₀₉₋₂₁₇-hβ₂m-A2 one to drive human DC maturation cultured ex-vivo in the absence of the maturation cocktail. To this end we transfected immature human DCs with both constructs, or treated the same cells with LPS (as a negative control) and subjected these cells, along with immature and mature DCs to flow cytometry analysis for expression of surface DC86 as a representative marker for maturation. FIG. 17 indeed reveals an elevated CD86 expression in cells transfected with gp100₂₀₉₋₂₁₇-hβ₂m-TLR4 compared with cells expressing gp100₂₀₉₋₂₁₇-hβ₂m-A2 or LPS-treated cells, which is indicative of the anticipated adjuvant function of the TLR4 moiety in this construct.

### Example 12

### Expression of hβ₂m-CD40 in APCs

We evaluated the adjuvant capacity of the CD40 activation domain a peptide-less β₂m context. The tm+cyt portion of mouse CD40 was cloned by RT-PCR from mRNA of A20, a mouse B cell lymphoma expressing CD40. The forward primer was: 5′CCC TCG AGC TCC ACT GTC TCC AAC ATG GCC CTG CTG GTC ATT CCT G 3′ (SEQ ID NO: 63) (with an XhoI restriction site) and the reverse primer was 5′CGC GCG GCC GCG GTC AGC AAG CAG CCA TC 3′ (SEQ ID NO: 64) (with a NotI site). The DNA product was cloned in the context of membranal human β₂m.

### Example 13

### The CD40 Activation Domain is Functional in A20 Transfectants

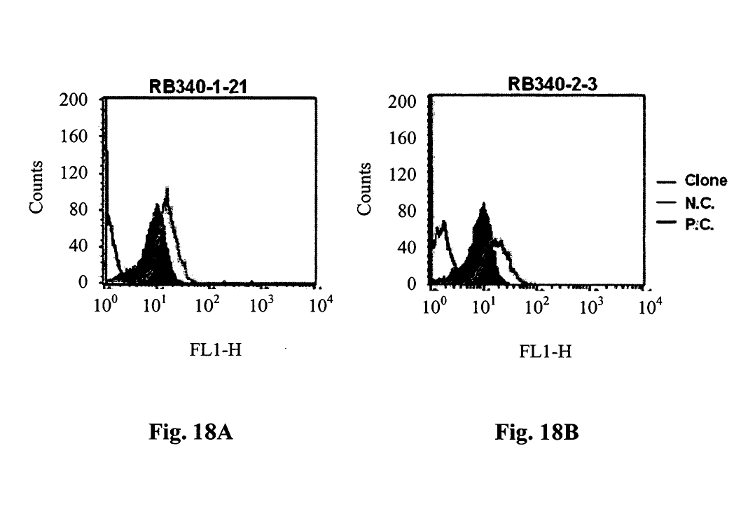

The A20 B cell lymphoma constitutively expresses CD40 and is readily activated by agonistic anti-CD40 antibodies (or a soluble form of the CD40 ligand). To evaluate the function of the appended CD40 moiety we performed a preliminary functional analysis in the A20 transfectants RB340-1-21 and RB340-2-3, expressing the monomeric hβ₂m-CD40 construct (FIGS. 18A-B show surface hP₂m expression by these two clones). Our assay was based on the natural pathway of CD40-mediated signaling in APCs. One of the downstream events in this pathway is the phosphorylation of IκB, the inhibitor of the NF-κB, which triggers its ubiquitination and subsequent proteasomal degradation. As a result of this process, the total level of IκB is substantially reduced shortly after activation and is only restored several hours later.

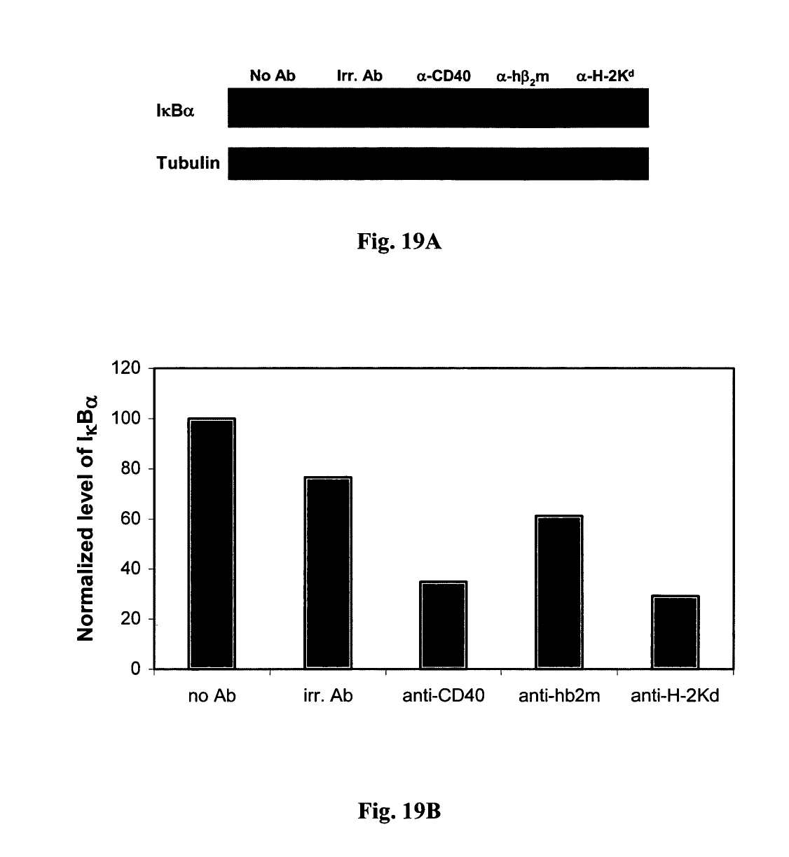

Antibodies against the α chain of IκB (IκBα) therefore serve as a useful analytical tool for CD40-mediated signaling. We have established an immunoblot-based assay, in which we evaluate total cellular IκBα level following activation, by normalizing it against the level of tubulin (or actin) as non-responsive housekeeping gene products. Cells were incubated for 1 hour with the indicated antibodies (Abs): hamster anti-mouse CD40 (Biolegend), mouse anti-hβ₂m (Sigma), mouse anti-H-2 K^{d} (InVitrogen) and then harvested. A calibrated amount of detergent lysates were subjected to PAGE and subsequent immunoblot analysis, first with an anti-IκBα mAb (Santa Cruz) and then, following stringent stripping of bound Abs, with an anti-mouse tubulin mAb (Santa Cruz). The results of a typical experiment are shown in FIG. 19. Total level of IκBα under non-stimulatory conditions is considered 100%. Two hours incubation with an irrelevant Ab results in a small (yet unexpected) decrease of 23%. However, a marked 71% reduction is observed following incubation with an anti-H-2 K^{d} mAb, which is comparable to the expected 65% reduction mediated by the agonistic anti-CD40 mAb. Importantly, this finding is also indicative of functional association between the hβ₂m-CD40 polypeptide products with MHC-I heavy chains at the cell surface. Interestingly, a mAb against hβ₂m led to a moderate 39% decrease in the total level of cellular IκBα, suggesting that under the same experimental conditions this mAb is only partially agonistic. Parental A20 cells responded similarly to the anti-CD40 mAb, but not to the other two (data not shown).

### Example 14

### Production of Peptide-β₂m-TLR-CD40 Constructs

All gene elements encoding the different components of this construct are assembled so as to preserve an open reading frame. To this end, we synthesize two new oligonucleotide primers to serve for overlapping PCR cloning of the TLR4-CD40 stretch, in which the last codon of TLR4 is followed by the codon of the first intracellular residue of CD40. The same forward primer we used for TLR4 (with an XhoI or SalI site) and the reverse CD40 we used for CD40 (with a NotI site) serve us for PCR amplification of the entire segment following the overlapping reactions.

### Example 15

### Functional Evaluation of the Genetic Adjuvants

Function of the incorporated genetic adjuvants in the context of vaccines are evaluated in ex-vivo immunization experiments using human samples, as described in part in Example 11 above. In these experiments, blood samples from healthy HLA-A2⁺ donors and melanoma biopsies and blood samples obtained from HLA-A2⁺ patients are used. Two HLA-A2 binding peptides derived from well-characterized melanoma-associated antigens are investigated: gp100₂₀₉₋₂₁₇ and MART1₂₇₋₃₅. A third HLA-A2-binding peptide, derived from the breast-tumor associated MUC1 protein (MUC1/D6), serve as a negative control. All these are cloned in the context of β₂m-TLR, β₂m-CD40, β₂m-TLR-CD40 or β₂m-A2 (the latter serving as a reference inert anchor) in the pEGM4Z/A64 vector. Endotoxin-free DNA templates are prepared and mRNA is synthesized in-vitro. Blood samples are thawed, DCs are propagated and RNA is transfected essentially as described above in ‘Materials and Methods for Examples 7-11’. In the following experiments, we shall use immature DCs to examine the function of the TLR component, as the DC maturation cocktail drives DC differentiation and activation in much the same route. To monitor the function of CD40, we transfect both immature and mature DCs. In all these experiments, the corresponding RNA harboring the same antigenic peptide but the HLA-A2-derived anchor is used as a reference for the function of the TLR and CD40 activation domains. To evaluate the dual function of the β₂m-TLR-CD40 construct, we compared it to the corresponding constructs harboring either the TLR or the CD40 portion separately.

To detect peptide presentation on transfected hDCs, gp100₂₀₉₋₂₁₇ or MART-1₂₇₋₃₅ HLA-A2⁺ Ag-specific T cell clones are co-incubated with modified HLA-A2+ DC. 0.5-1×10⁵ cells/well of both stimulators and responders are co-cultured for 24 hours in a 96-well plate, at a ratio of 1:1. Supernatants are collected after co-incubation for determination of human IFN-γ secretion by the T cells clones using a commercial ELISA kit. Alternatively, monoclonal phage antibodies specific for the same complexes (a kind gift from Dr. Y. Reiter, Technion, Haifa, Israel) are used in flow cytometry analysis of the transfected DCs.

To analyze the maturation profile of the transfected DCs we use RT-PCR and flow cytometry protocols, which are similar to those described in Examples 14, 15 and 17 above.

For ex-vivo priming of donor's or patients' CTLs, non-adherent cells from blood samples are enriched for CD8 T cells by magnetic bead depletion using a panel of mAbs. At day 0, purified CD8 T cells are co-incubated with irradiated RNA-transfected DCs at a ratio of 5:1 responders to stimulators. Stimulators also include peptide-pulsed DCs (at 50 μg/ml) as controls. Medium is replaced and IL-2 at 30 IU/ml is added to culture at 2-day intervals till day 12. TAA-mediated cytokine release by stimulated CD8⁺ T cells is assayed by IFN-γ ELISA following co-cultures of day 14 CD8⁺ T cells with the following target cells: Autologous transfected or peptide-loaded DCs, HLA-A2⁺/gp100⁺ or MART-1⁺ melanoma cells and peptide-loaded T2 cells. Non-modified autologous DCs, HLA-A2⁻ melanoma lines and an irrelevant HLA-A2-restricted peptide loaded onto T2 cells are used as controls. For these assays, 5×10⁵ responder cells and 5×10⁴ stimulator cells are incubated overnight in a 0.2 ml complete medium in individual wells of 96-well plates. Secretion of additional cytokines (IL-2, IL-4, IL-10, GM-CSF), to define Th1 or Th2 type immune responses, are monitored by ELISA.

Peptide-specific cytolysis of target cells is then be determined. At day 14, re-stimulated CD8 T cells are harvested and assayed in a standard ⁵¹Cr release assay at a series of effector:target ratios against the following target cells: Autologous peptide-loaded DCs, autologous melanoma cells (grown from biopsies) and peptide loaded T2 cells.77

### REFERENCES

Andersen, P. S., Stryhn, A., Hansen, B. E., Fugger, L., Engberg, J., and Buus, S. 1996. A recombinant antibody with the antigen-specific, major histocompatibility complex-restricted specificity of T cells. Proc Natl Acad Sci U S A 93:1820-4.
Armstrong, T. D., Pulaski, B. A., and Ostrand_Rosenberg, S. 1998. Tumor antigen presentation: changing the rules. Cancer Immunol. Immunother. 46:70-4.
Benton, P. A. and Kennedy, R. C. 1998. DNA vaccine strategies for the treatment of cancer. Curr Top Microbiol Immunol 226:1-20.
Berger, C. L., Longley, B. J., Imaeda, S., Christensen, I., Heald, P., and Edelson, R. L. 1998. Tumor-specific peptides in cutaneous T-cell lymphoma: association with class I major histocompatibility complex and possible derivation from the clonotypic T-cell receptor. Int J Cancer 76:304-11.
Berko, D., Y. Carmi, G. Cafri, S. Ben-Zaken, H. M. Sheikhet, E. Tzehoval, L. Eisenbach, A. Margalit, and G. Gross. 2005. Membrane-Anchored {beta}2-Microglobulin Stabilizes a Highly Receptive State of MHC Class I Molecules. J Immunol 174:2116-2123.
Bloom, M. B., Perry-Lalley, D., Robbins, P. F., Li, Y., el-Gamil, M., Rosenberg, S. A., and Yang, J. C. 1997. Identification of tyrosinase-related protein 2 as a tumor rejection antigen for the B16 melanoma. J Exp Med 185:453-9.
Bubenik, J. 2001. Genetically engineered dendritic cell-based cancer vaccines (review). Int J. Oncol. 18:475-8.
Cisco, R. M., Z. Abdel-Wahab, J. Dannull, S. Nair, D. S. Tyler, E. Gilboa, J. Vieweg, Y. Daaka, and S. K. Pruitt. 2004. Induction of human dendritic cell maturation using transfection with RNA encoding a dominant positive toll-like receptor 4. J Immunol 172:7162-7168.
Chen, W., Rains, N., Young, D., and Stubbs, R. S. 2000. Dendritic cell-based cancer immunotherapy: potential for treatment of colorectal cancer? J Gastroenterol Hepatol 15:698-705.
Chen, S-S., Gong, J., Yang, Y-M., Oettge, H. and Zanetti, M., 2005. Cytotoxic T-cells specific for natural IgE peptides downregulate IgE production. Cell. Immunol. 233: 11-22.
Choi, M. S., Brines, R. D., Holman, M. J., and Klaus, G. G. 1994. Induction of NFAT in normal B lymphocytes by anti-immunoglobulin or CD40 ligand in conjunction with IL-4. Immunity 1: 179-87.
Cohen, I. R. and Weiner, H. L. 1988. T-cell vaccination. Immunol Today 9:332-5.
Cortesini, R., LeMaoult, J., Ciubotariu, R. and Cortesini, N. S., 2001. CD8+CD28− T suppressor cells and the induction of antigen-specific, antigen-presenting cell-mediated suppression of Th reactivity. Immunol. Rev. 182: 201-206.
Dalgleish, A. G. 2001. Current problems in the development of specific immunotherapeutic approaches to cancer. J. Clin. Pathol. 54:675-6.
Feldman M. and Eisenbach L. 1991. MHC class I genes controlling the metastatic phenotype of tumor cells. Semin Cancer Biol. Oct;2(5):337-46:
Eshhar, Z., T. Waks, A. Bendavid, and D. G. Schindler. 2001. Functional expression of chimeric receptor genes in human T cells. J Immunol Methods 248:67-76.
Gervois, N., Guilloux, Y., Diez, E., and Jotereau, F. 1996. Suboptimal activation of melanoma infiltrating lymphocytes (TIL) due to low avidity of TCR/MHC-tumor peptide interactions. J. Exp. Med. 183:2403-7.
Gilboa, E., Nair, S. K., and Lyerly, H. K. 1998. Immunotherapy of cancer with dendritic-cell-based vaccines. Cancer Immunol, Immunother 46:82-7.
Gilboa, E. 1999. The makings of a tumor rejection antigen. Immunity 11:263-70.
Gong, J., Chen, D., Kashiwaba, M., and Kufe, D. 1997. Induction of antitumor activity by immunization with fusions of dendritic and carcinoma cells. Nature Medicine 3:558-61.
Gong, J., Nikrui, N., Chen, D., Koido, S., Wu, Z., Tanaka, Y., Cannistra, S., Avigan, D., and Kufe, D. 2000. Fusions of human ovarian carcinoma cells with autologous or allogeneic dendritic cells induce antitumor immunity. J Immunol 165:1705-11.
Guermonprez, P., Valladeau, J., Zitvogel, L., Thery, C., and Amigorena, S. 2002. Antigen presentation and T cell stimulation by dendritic cells. Annu Rev Immunol 20:621-67.
Gurunathan, S., Klinman, D. M., and Seder, R. A. 2000. DNA vaccines: immunology, application, and optimization*. Annu Rev Immunol 18:927-74.
Hadzantonis, M. and O'Neill, H.1999. Review: dendritic cell immunotherapy for melanoma. Cancer Biother Radiopharm. 14:11-22.
Heath, W. R. and Carbone, F. R. 2001. Cross-presentation, dendritic cells, tolerance and immunity. Annual Review of Immunology 19:47-64.
Horig, H., Young, A. C., Papadopoulos, N. J., DiLorenzo, T. P., and Nathenson, S. G. 1999. Binding of longer peptides to the H-2Kb heterodimer is restricted to peptides extended at their C terminus: refinement of the inherent MHC class I peptide binding criteria. J Immunol 163:4434-41.
Huang, C. H.; Peng, S.; He, L.; Tsai, Y. C.; Boyd, D. A.; Hansen, T. H.; Wu, T. C. and Hung, C. F., 2005. Cancer immunotherapy using a DNA vaccine encoding a single-chain trimer of MHC class I linked to an HPV-16 E6 immunodominant CTL epitope. Gene Ther., 12(15), 1180-1186.
Howell, M. D., Winters, S. T., Olee, T., Powell, H. C., Carlo, D. J. and Brostoff, S. W (1989) Vaccination against experimental allergic encephalomyelitis with T cell receptor peptides. Science 246(4930), 668-670
Huseby, E. S., Liggitt, D., Brabb, T., Schnabel, B., Ohlen, C., and Goverman, J., 2001. A pathogenic role for myelin-specific CD8(+) T cells in a model for multiple sclerosis. J. Exp. Med. 194, 669-76.
Jager, E., Jager, D., and Knuth, A. 2002. Clinical cancer vaccine trials. Curr Opin Immunol 14:178-82.
Janeway, C. A. and Bottomly, K. 1994. Signals and signs for lymphocyte responses. Cell 76:275-85.
Kaufmann, Y., Berke, G., and Eshhar, Z. 1981. Cytotoxic T lymphocyte hybridomas that mediate specific tumor-cell lysis in vitro. Proc Natl Acad Sci USA 78:2502-6.
Kawakami, Y., S. Eliyahu, C. Jennings, K. Sakaguchi, X. Kang, S. Southwood, P. F. Robbins, A. Sette, E. Appella, and S. A. Rosenberg. 1995. Recognition of multiple epitopes in the human melanoma antigen gp100 by tumor-infiltrating T lymphocytes associated with in vivo tumor regression. J. Immunol. 154:3961-3968.
Kumar, V. and Sercarz, E. 2001. An integrative model of regulation centered on recognition of TCR peptide/MHC complexes. Immunol Rev 182:113-21.
Kumar, V., Tabibiazar, R., Geysen, H. M., and Sercarz, E. 1995. Immunodominant framework region 3 peptide from TCR V beta 8.2 chain controls murine experimental autoimmune encephalomyelitis. J Immunol 154:1941-50.
Levitsky, V., Zhang, Q. J., Levitskaya, J., and Masucci, M. G. 1996. The life span of major histocompatibility complex-peptide complexes influences the efficiency of presentation and immunogenicity of two class I-restricted cytotoxic T lymphocyte epitopes in the Epstein-Barr virus nuclear antigen 4. Jf Exp Med 183:915-26.
Liblau, R. S., Wong, F. S., Mars, L. T. and Santamaria, P., 2002. Autoreactive CD8 T cells in organ-specific autoimmunity: emerging targets for therapeutic intervention. Immunity 17(1) 1-6;.
Liu H, Komai-Koma M, Xu D, and Liew FY. 2006. Toll-like receptor 2 signaling modulates the functions of CD4+CD25+ regulatory T cells. Proc Natl Acad Sci USA. 103, 7048-53.
Liu H & Leung BP. 2006. CD4+CD25+ regulatory T cells in health and disease. Clin Exper Pharmacol Physiol 33: 519-24.
Lone, Y. C., Motta, I., Mottez, E., Guilloux, Y., Lim, A., Demay, F., Levraud, J. P., Kourilsky, P., and Abastado, J. P. 1998. In vitro induction of specific cytotoxic T lymphocytes using recombinant single-chain MHC class I/peptide complexes. J Immunother 21:283-94.
Lustgarten J, Eshhar Z., 1995. Specific elimination of IgE production using T cell lines expressing chimeric T cell receptor genes Eur J. Immunol. 25:2985-91.
Lybarger, L.; Yu, Y. Y.; Miley, M. J.; Fremont, D. H.; Myers, N.; Primeau, T.; Truscoff, S. M.; Connolly, J. M. and Hansen, T. H. (2003) Enhanced Immune Presentation of a Single-chain Major Histocompatibility Complex Class I Molecule Engineered to Optimize Linkage of a C-terminally Extended Peptide J. Immunol. J. Biol. Chem., 278(29), 27105-27111;
Mahnke, K., Qian, Y., Knop, J. and Enk, A. H., 2003. Induction of CD4+/CD25+ regulatory T cells by targeting of antigens to immature dendritic cells. Blood 101(12), 4862-4869.
Margalit, A., H. M. Sheikhet, Y. Carmi, D. Berko, E. Tzehoval, L. Eisenbach, and G. Gross. 2006. Induction of Antitumor Immunity by CTL Epitopes Genetically Linked to Membrane-Anchored {beta}2-Microglobulin. J. Immunol. 176:217-224.
Margalit, A., S. Fishman, D. Berko, J. Engberg, and G. Gross. 2003. Chimeric beta2 microglobulin/CD3zeta polypeptides expressed in T cells convert MHC class I peptide ligands into T cell activation receptors: a potential tool for specific targeting of pathogenic CD8(+) T cells. Int Immunol 15:1379-1387.
Medzhitov, R., P. Preston-Hurlburt, and C. A. Janeway, Jr. 1997. A human homologue of the Drosophila Toll protein signals activation of adaptive immunity. Nature 388:394-397.
Melian A, Geng Y J, Sukhova G K, Libby P, and S A Porcelli. 1999. CD1 expression in human atherosclerosis. A potential mechanism for T cell activation by foam cells. Am J. Pathol. 155:775-86.
Minev, B. R., Chavez, F. L., and Mitchell, M. S. 1999. Cancer vaccines: novel approaches and new promise. Pharmacol and Therapeut 81:121-39Moingeon, P. 2001. Cancer vaccines. Vaccine 19:1305-26.
Moingeon, P. 2001. Cancer vaccines. Vaccine 19:1305-26
Mottez, E., Langlade_Demoyen, P., Gournier, H., Martinon, F., Maryanski, J., Kourilsky, P., and Abastado, J. P. 1995. Cells expressing a major histocompatibility complex class I molecule with a single covalently bound peptide are highly immunogenic. J Exp Med 181:493-502.
Nilsson J, Hansson G K, and P K Shah. 2005. Immunomodulation of Atherosclerosis: Implications for Vaccine Development. Arterioscler. Thromb. Vasc. Biol; 25; 18-28.
Nouri-Shirazi, M., Banchereau, J., Fay, J., and Palucka, K. 2000. Dendritic cell based tumor vaccines. Immunol Letters 74:5-10
Ojcius, D. M., Langlade-Demoyen, P., Gachelin, G., and Kourilsky, P. 1994. Role for MHC class I molecules in selecting and protecting high affinity peptides in the presence of proteases. J Immunol 152:2798-810.
Offner, H., Jacobs, R., Bebo, B. F., Jr., and Vandenbark, A. A. 1999. Treatments targeting the T cell receptor (TCR): effects of TCR peptide-specific T cells on activation, migration, and encephalitogenicity of myelin basic protein-specific T cells. Springer Semin Immunopathol 21:77-90.
Parker, K. C., Bednarek, M. A., and Coligan, J. E., 1994. Scheme for ranking potential HLA-A2 binding peptides based on independent binding of individual peptide side-chains. J. Immunol. 152, 163-75.
Pasare, C., and R. Medzhitov. 2003. Toll pathway-dependent blockade of CD4+CD25+ T cell-mediated suppression by dendritic cells. Science 299:1033-1036. Epub 2003 Jan 1016.
Porgador, A., Snyder, D., and Gilboa, E. 1996. Induction of antitumor immunity using bone marrow-generated dendritic cells. J Immunol 156-2918-26.
Porgador, A., Feldman, M., and Eisenbach, L. 1989. H-2Kb transfection of B16 melanoma cells results in reduced tumourigenicity and metastatic competence. J Immunogen 16:291-303.s Porgador, A., Yewdell, J. W., Deng, Y., Bennink, J. R., and Germain, R. N. 1997. Localization, quantitation, and in situ detection of specific peptide-MHC class I complexes using a monoclonal antibody. Immunity 6:715-26
Porgador, A., Irvine, K. R., Iwasaki, A., Barber, B. H., Restifo, N. P., and Germain, R. N. 1998. Predominant role for directly transfected dendritic cells in antigen presentation to CD8+ T cells after gene gun immunization. J Exp Med 188:1075-82.
Quinn, A., McInerney, M. F., and Sercarz, E. E., 2001. MHC class I-restricted determinants on the glutamic acid decarboxylase 65 molecule induce spontaneous CTL activity. J. Immunol. 167, 1748-57.
Rammensee, H., Bachmann, J., Emmerich, N. P., Bachor, O. A., and Stevanovic, S., 1999. SYFPEITHI: database for MHC ligands and peptide motifs. Immunogenetics 50, 213-9.
Re, F. and Strominger, J. L. 2001. Toll-like receptor 2 (TLR2) and TLR4 differentially activate human dendritic cells. J. Biol. Chem. 276(40), 37692-37699).
Reis e Sousa, C. 2001. Dendritic cells as sensors of infection. Immunity 14:495-8.
Rosenberg, S. A. 1999. A new era of cancer immunotherapy: converting theory to performance. CA Cancer J. Clin. 49:70-3, 65.
Rosenberg, S. A., J. C. Yang, and N. P. Restifo. 2004. Cancer immunotherapy: moving beyond current vaccines. Nat Med 10:909-915.
Sanderson, S. and Shastri, N. 1994. LacZ inducible, antigen/MHC-specific T cell hybrids. Int Immunol 6:369-76.
Saric, T., Chang, S. C., Hattori, A., York, I. A., Markant, S., Rock, K. L., Tsujimoto, M., and Goldberg, A. L. 2002. An IFN-gamma-induced aminopeptidase in the ER, ERAPI, trims precursors to MHC class I-presented peptides. Nat Immunol 3:1169-76.
Serwold, T., Gonzalez, F., Kim, J., Jacob, R., and Shastri, N. 2002. ERAAP customizes peptides for MHC class I molecules in the endoplasmic reticulum. Nature 419:480-3. Pulendran, B. 2004. Modulating vaccine responses with dendritic cells and Toll-like receptors. Immunol Rev 199:227-250.
Sherritt, M., Cooper, L., Moss, D. J., Kienzle, N., Altman, J., and Khanna, R. 2001. Immunization with tumor-associated epitopes fused to an endoplasmic reticulum translocation signal sequence affords protection against tumors with down-regulated expression of MHC and peptide transporters. Int Immunol 13:265-71.
Snyder, H. L., Yewdell, J. W., and Bennink, J. R. 1994. Trimming of antigenic peptides in an early secretory compartment. J Exp Med 180:2389-94.
Snyder, H. L., Bacik, I., Yewdell, J. W., Behrens, T. W., and Bennink, J. R. 1998. Promiscuous liberation of MHC-class I-binding peptides from the C termini of membrane and soluble proteins in the secretory pathway. Eur J Immunol 28:1339-46.
Sogn, J. A. 1998. Tumor immunology: the glass is half full. Immunity 9:757-63.
Sogn, J. A. 2000. The status of tumor immunology and cancer immunotherapy. Introduction. Immunol Investig 29:81-4.
Steinman, R. M. 1989. Dendritic cells: clinical aspects. Res Immunol 140:911-8; discussion 918-26.
Steinman, L., 2001. J. Exp. Med. 194, 27-30.
Stryhn, A., Pedersen, L. O., Holm, A., and Buus, S. 2000. Longer peptide can be accommodated in the MHC class I binding site by a protrusion mechanism. Eur J Immunol 30:3089-99.
Sun, D., Whitaker, J. N., Huang, Z., Liu, D., Coleclough, C., Wekerle, H., and Raine, C. S., 2001. Myelin antigen-specific CD8+ T cells are encephalitogenic and produce severe disease in C57BL/6 mice. J. Immunol. 166: 7579-87.
Tafuro, S., Meier, U. C., Dunbar, P. R., Jones, E. Y., Layton, G. T., Hunter, M. G., Bell, J. I., and McMichael, A. J. 2001. Reconstitution of antigen presentation in HLA class I-negative cancer cells with peptide-beta2m fusion molecules. Eur J Immunol 31:440-9.
Takahashi, H., Nakagawa, Y., Yokomuro, K., and Berzofsky, J. A. 1993. Induction of CD8+ cytotoxic T lymphocytes by immunization with syngeneic irradiated HIV-1 envelope derived peptide-pulsed dendritic cells. Int Immunol 5:849-57.
Tang Q & Bluestone JA. 2006. Regulatory T-cell physiology and application to treat autoimmunity. Immunol Rev 212: 217-37,
Tirosh, B., el_Shami, K., Vaisman, N., Carmon, L., Bar_Haim, E., Vadai, E., Feldman, M., Fridkin, M., and Eisenbach, L. 1999. Immunogenicity of H-2Kb-low affinity, high affinity, and covalently-bound peptides in anti-tumor vaccination. Immunol Letters 70:21-8.
Tsomides, T. J., Aldovini, A., Johnson, R. P., Walker, B. D., Young, R. A., and Eisen, H. N. 1994. Naturally processed viral peptides recognized by cytotoxic T lymphocytes on cells chronically infected by human immunodeficiency virus type 1. J Exp Med 180:1283-93.
Uger, R. A. and Barber, B. H.1998. Creating CTL targets with epitope-linked beta 2-microglobulin constructs. J Immunol 160:1598-605.
Uger, R. A., Chan, S. M., and Barber, B. H.1999. Covalent linkage to beta2-microglobulin enhances the MHC stability and antigenicity of suboptimal CTL epitopes. J Immunol 162:6024-8.
Vora, A. R., Rodgers, S., Parker, A. J., Start, R., Rees, R. C., and Murray, A. K. 1997. An immunohistochemical study of altered immunomodulatory molecule expression in head and neck squamous cell carcinoma. Brit J Cancer 76:836-44.
Walsh P T, Taylor D K, and Turka L A. 2004. Tregs and transplantation tolerance. J. Clin. Invest. 114: 1398-403.
Wang, R. F., and H. Y. Wang. 2002. Enhancement of antitumor immunity by prolonging antigen presentation on dendritic cells. Nat Biotechnol 20:149-154.
Watson, G. A. and Lopez, D. M. 1995. Aberrant antigen presentation by macrophages from tumor-bearing mice is involved in the down-regulation of their T cell responses. J Immunol 155:3124-34.
Wen, Y. J. and Lim, S. H.1997. T cells recognize the VH complementarity-determining region 3 of the idiotypic protein of B cell non-Hodgkin's lymphoma. Eur J Immunol 27:1043-7.
White, J., Crawford, F., Fremont, D., Marrack, P., and Kappler, J. 1999. Soluble class I MHC with beta2-microglobulin covalently linked peptides: specific binding to a T cell hybridoma. J Immunol 162:2671-6.
Wong, F. S., Karttunen, J., Dumont, C., Wen, L., Visintin, I., Pilip, I. M., Shastri, N., Pamer, E. G., and Janeway, C. A., Jr., 1999. Identification of an MHC class I-restricted autoantigen in type 1 diabetes by screening an organ-specific cDNA library. Nat. Med. 5, 1026-31.
Yang, Y., C. T. Huang, X. Huang, and D. M. Pardoll. 2004. Persistent Toll-like receptor signals are required for reversal of regulatory T cell-mediated CD8 tolerance. Nat Immunol 5:508-515. Epub 2004 April 2004.
York, I. A., Chang, S. C., Saric, T., Keys, J. A., Favreau, J. M., Goldberg, A. L., and Rock, K. L. 2002. The ER aminopeptidase ERAPI enhances or limits antigen presentation by trimming epitopes to 8-9 residues. Nat Immunol 3:1177-84.
Yu, Y. Y.; Netuschil, N.; Lybarger, L.; Connolly, J. M. and Hansen, T. H. (2002), Cutting Edge: Single-Chain Trimers of MHC Class I Molecules Form Stable Structures That Potently Stimulate Antigen-Specific T Cells and B Cells. 168(7), 3145-3149
Zhang, W., Cao, X., and Huang, X. 1997. [In vivo induction of antitumor immune response by tumor cells fused with GM-CSF gene-modified dendritic cells]. Zhonghua Yi Xue Za Zhi. (Chinese). 77:39-42.
Zwar T D, VAN Driel I R, and Gleeson P A. 2006. Guarding the immune system: Suppression of autoimmunity by CD4CD25 immunoregulatory T cells. Immunol Cell Biol. Sep 5, [Epub ahead of print].

1. A polynucleotide comprising a sequence encoding a polypeptide that is capable of high level presentation of antigenic peptides on antigen-presenting cells, wherein the polypeptide comprises a β2-microglobulin molecule that is linked through its carboxyl terminal to a polypeptide stretch that allows the anchorage of the β2-microglobulin molecule to the cell membrane, and through its amino terminal to at least one antigenic peptide comprising an MHC class I epitope, and said polypeptide stretch consists of a bridge peptide that spans the whole distance to the cell membrane, said bridge peptide being linked to the full or partial transmembrane and/or cytoplasmic domains of a molecule selected from the group consisting of a toll-like receptor (TLR) polypeptide, a CD40 polypeptide, and a TLR polypeptide and a CD40 polypeptide fused in tandem.

2. The polynucleotide of claim 1, wherein said bridge peptide is linked to the full or partial transmembrane and/or cytoplasmic domains of a TLR polypeptide.

3. The polynucleotide of claim 2, wherein said TLR polypeptide is selected from the group consisting of TLR 1, 2, 3, 4, 5, 6, 7, 8 and 9.

4. The polynucleotide of claim 3, wherein said TLR polypeptide is the human TLR4.

5. The polynucleotide of claim 3, wherein said TLR polypeptide is the human TLR2.

6. The polynucleotide of claim 1, wherein said bridge peptide is linked to the full or partial transmembrane and/or cytoplasmic domains of a CD40 polypeptide.

7. The polynucleotide of claim 1, wherein said bridge peptide is linked to the full or partial transmembrane and/or cytoplasmic domains of a CD40 and a TLR polypeptide fused in tandem.

8. The polynucleotide of claim 1, wherein said bridge peptide is the peptide of SEQ ID NO: 1.

9. The polynucleotide of claim 1, wherein said at least one antigenic peptide comprising an MHC class I epitope is linked to the β2-microglobulin amino terminal through a peptide linker.

10. The polynucleotide of claim 1, wherein said at least one antigenic peptide is at least one antigenic determinant of one sole antigen.

11. The polynucleotide of claim 1, wherein said at least one antigenic peptide is at least one antigenic determinant of each one of at least two different antigens.

12. The polynucleotide of claim 1, wherein said at least one antigenic peptide has a sequence derived from a tumor-associated antigen (TAA).

13. The polynucleotide of claim 12, wherein said TAA is selected from the group consisting of alpha-fetoprotein, BA-46/lactadherin, BAGE, BCR-ABL fusion protein, beta-catenin, CASP-8, CDK4, CEA, CRIPTO-1, elongation factor 2, ETV6-AML1 fusion protein, G250, GAGE, gp100, HER-2/neu, intestinal carboxyl esterase, KIAA0205, MAGE, MART-1/Melan-A, MUC-1, N-ras, p53, PAP, PSA, PSMA, telomerase, TRP-1/gp75, TRP-2, tyrosinase, and uroplakin Ia, Ib, II and III.

14. The polynucleotide of claim 13, wherein the at least one antigenic peptide is selected from the group consisting of:
(i) the alpha-fetoprotein peptide GVALQTMKQ (SEQ ID NO:4);
(ii) the BAGE-1 peptide AARAVFLAL (SEQ ID NO:5);
(iii) the BCR-ABL fusion protein peptide SSKALQRPV (SEQ ID NO:6);
(iv) the beta-catenin peptide SYLDSGIHF (SEQ ID NO:7);
(v) the CDK4 peptide ACDPHSGHFV (SEQ ID NO:8);
(vi) the CEA peptide YLSGANLNL (SEQ ID NO:9);
(vii) the elongation factor 2 peptide ETVSEQSNV (SEQ ID NO: 10);
(viii) the ETV6-AML 1 fusion protein peptide RIAECILGM (SEQ ID NO:11)
(ix) the G250 peptide HLSTAFARV (SEQ ID NO: 12);
(x) the GAGE-1,2,8 peptide YRPRPRRY (SEQ ID NO:13)
(xi) the gp100 peptide KTWGQYWQV (SEQ ID NO: 14),
(xii) (A)MLGTHTMEV (SEQ ID NO: 15), ITDQVPFSV (SEQ ID NO:16), YLEPGPVTA (SEQ ID NO:17), LLDGTATLRL (SEQ ID NO:18), VLYRYGSFSV (SEQ ID NO:19), SLADTNSLAV (SEQ ID NO:20), RLMKQDFSV (SEQ ID NO:21), RLPRIFCSC (SEQ ID NO:22), LIYRRRLMK (SEQ ID NO:23), ALLAVGATK (SEQ ID NO:24), IALNFPGSQK (SEQ ID NO:25) and ALNFPGSQK (SEQ ID NO:26);
(xiii) the HER-2/neu peptide KIFGSLAFL (SEQ ID NO:27);
(xiv) the intestinal carboxyl esterase peptide SPRWWPTCL (SEQ ID NO:28);
(xv) the KIAA0205 peptide AEPINIQTW (SEQ ID NO:29);
(xvi) the MAGE-1 peptides EADPTGHSY (SEQ ID NO:30) and SLFRAVITK (SEQ ID NO:31);
(xvii) the MAGE-3 peptides EVDPIGHLY (SEQ ID NO:32) and FLWGPRALV (SEQ ID NO:33);
(xviii) the MART-1/Melan-A peptide (E)AAGIGILTV (SEQ ID NO:34);
(xix) the MUC-1 peptide STAPPVHNV (SEQ ID NO:35);
(xx) the N-ras peptide ILDTAGREEY (SEQ ID NO:36);
(xxi) the p53 peptide LLGRNSFEV (SEQ ID NO:37);
(xxii) the PSA peptides FLTPKKLQCV (SEQ ID NO:38) and VISNDVCAQV (SEQ ID NO:39);
(xxiii) the telomerase peptide ILAKFLHWL (SEQ ID NO:40);
(xxiv) the TRP-1 peptide MSLQRQFLR (SEQ ID NO:41);
(xxv) the TRP-2 peptides LLGPGRPYR (SEQ ID NO:42), SVYDFFVWL (SEQ ID NO:43), and TLDSQVMSL (SEQ ID NO:44);
(xxvi) the TRP2-INT2 peptide EVISCKLIKR (SEQ ID NO:45); and
(xxvii) the tyrosinase peptide KCDICTDEY (SEQ ID NO:46).

15. The polynucleotide of claim 12, wherein the at least one antigenic peptide derived from a TAA is at least one antigenic determinant of each one of at least two different TAAs.

16. The polynucleotide of claim 15, wherein said at least one antigenic peptide is at least one HLA-A2 binding peptide derived from each one of the melanoma associated antigens gp100 and Melan-A/MART-1, or said at least one antigenic peptide is at least one HLA-A3-restricted gp100 and at least one HLA-A2-restricted Melan-A/MART-1 peptide.

17. The polynucleotide of claim 16, wherein the at least one antigenic peptide is at least one HLA-A2 binding peptide and at least one HLA-A3 binding peptide derived from the melanoma-associated antigen gp100, and said at least one HLA-A2 binding peptide derived from gp100 is selected from the group consisting of SEQ ID NO: 14, 15, 16, 17, 18, 19, 20, 21 and 22, and said at least one gp100 HLA-A3 binding peptide is selected from the group consisting of SEQ ID NO: 23, 24, 25 and 26.

18. The polynucleotide of claim 10, wherein said antigen is an antigen from a pathogen selected from the group consisting of a bacterial, viral, fungal and parasite antigen, or a membrane-bound IgE molecule antigen.

19. A polynucleotide of claim 1, wherein said antigenic peptide is related to an autoimmune disease.

20. An expression vector comprising a polynucleotide according to claim 1.

21. A cell that expresses a polypeptide encoded by a polynucleotide of claim 1.

22. A cell of claim 21 being an antigen-presenting cell selected from the group consisting of a dendritic cell, a macrophage, a B cell and a fibroblast, or an immune cell selected from the group consisting of T helper cells (CD4⁺), T regulatory cells (Treg; CD4⁺CD25⁺), cytotoxic T lymphocytes (CD8⁺) and natural killer (NK) cells, capable of recognizing and binding to harmful T cells and causing their elimination or inactivation.

23. A DNA vaccine comprising a polynucleotide of claim 1 or an expression vector of claim 20.

24. A cellular vaccine comprising an antigen-presenting cell of claim 22.

25. A method of immunizing a mammal against a tumor-associated antigen comprising the step of immunizing the mammal with a cellular vaccine that comprises an antigen presenting cell transfected with a polynucleotide of claim 1 wherein the β2-microglobulin molecule is linked through its amino terminal to at least one antigenic peptide having a sequence derived from at least one tumor-associated antigen.


SEQUENCE LISTING (89 sequences)

SEQ ID NO: 1 - protein, 13 aa, Artificial Sequence
  Synthetic
LRWEPSSQPTIPI

SEQ ID NO: 2 - protein, 57 aa, Artificial Sequence
  Synthetic
VGIIAGLVLFGAVITGAVVAAVMWRRKSSDRKGGSYSQAASSDSAQGSDVSLTACKV

SEQ ID NO: 3 - protein, 17 aa, Artificial Sequence
  Synthetic
FTLTGLLGTLVTMGLLT

SEQ ID NO: 4 - protein, 9 aa, Artificial Sequence
  Synthetic
GVALQTMKQ

SEQ ID NO: 5 - protein, 9 aa, Artificial Sequence
  Synthetic
AARAVFLAL

SEQ ID NO: 6 - protein, 9 aa, Artificial Sequence
  Synthetic
SSKALQRPV

SEQ ID NO: 7 - protein, 9 aa, Artificial Sequence
  Synthetic
SYLDSGIHF

SEQ ID NO: 8 - protein, 10 aa, Artificial Sequence
  Synthetic
ACDPHSGHFV

SEQ ID NO: 9 - protein, 9 aa, Artificial Sequence
  Synthetic
YLSGANLNL

SEQ ID NO: 10 - protein, 9 aa, Artificial Sequence
  Synthetic
ETVSEQSNV

SEQ ID NO: 11 - protein, 9 aa, Artificial Sequence
  Synthetic
RIAECILGM

SEQ ID NO: 12 - protein, 9 aa, Artificial Sequence
  Synthetic
HLSTAFARV

SEQ ID NO: 13 - protein, 8 aa, Artificial Sequence
  Synthetic
YRPRPRRY

SEQ ID NO: 14 - protein, 9 aa, Artificial Sequence
  Synthetic
KTWGQYWQV

SEQ ID NO: 15 - protein, 9 aa, Artificial Sequence
  Synthetic
XLGTHTMEV

SEQ ID NO: 16 - protein, 9 aa, Artificial Sequence
  Synthetic
ITDQVPFSV

SEQ ID NO: 17 - protein, 9 aa, Artificial Sequence
  Synthetic
YLEPGPVTA

SEQ ID NO: 18 - protein, 10 aa, Artificial Sequence
  Synthetic
LLDGTATLRL

SEQ ID NO: 19 - protein, 10 aa, Artificial Sequence
  Synthetic
VLYRYGSFSV

SEQ ID NO: 20 - protein, 10 aa, Artificial Sequence
  Synthetic
SLADTNSLAV

SEQ ID NO: 21 - protein, 9 aa, Artificial Sequence
  Synthetic
RLMKQDFSV

SEQ ID NO: 22 - protein, 9 aa, Artificial Sequence
  Synthetic
RLPRIFCSC

SEQ ID NO: 23 - protein, 9 aa, Artificial Sequence
  Synthetic
LIYRRRLMK

SEQ ID NO: 24 - protein, 9 aa, Artificial Sequence
  Synthetic
ALLAVGATK

SEQ ID NO: 25 - protein, 10 aa, Artificial Sequence
  Synthetic
IALNFPGSQK

SEQ ID NO: 26 - protein, 9 aa, Artificial Sequence
  Synthetic
ALNFPGSQK

SEQ ID NO: 27 - protein, 9 aa, Artificial Sequence
  Synthetic
KIFGSLAFL

SEQ ID NO: 28 - protein, 9 aa, Artificial Sequence
  Synthetic
SPRWWPTCL

SEQ ID NO: 29 - protein, 9 aa, Artificial Sequence
  Synthetic
AEPINIQTW

SEQ ID NO: 30 - protein, 9 aa, Artificial Sequence
  Synthetic
EADPTGHSY

SEQ ID NO: 31 - protein, 9 aa, Artificial Sequence
  Synthetic
SLFRAVITK

SEQ ID NO: 32 - protein, 9 aa, Artificial Sequence
  Synthetic
EVDPIGHLY

SEQ ID NO: 33 - protein, 9 aa, Artificial Sequence
  Synthetic
FLWGPRALV

SEQ ID NO: 34 - protein, 9 aa, Artificial Sequence
  Synthetic
XAGIGILTV

SEQ ID NO: 35 - protein, 9 aa, Artificial Sequence
  Synthetic
STAPPVHNV

SEQ ID NO: 36 - protein, 10 aa, Artificial Sequence
  Synthetic
ILDTAGREEY

SEQ ID NO: 37 - protein, 9 aa, Artificial Sequence
  Synthetic
LLGRNSFEV

SEQ ID NO: 38 - protein, 10 aa, Artificial Sequence
  Synthetic
FLTPKKLQCV

SEQ ID NO: 39 - protein, 10 aa, Artificial Sequence
  Synthetic
VISNDVCAQV

SEQ ID NO: 40 - protein, 9 aa, Artificial Sequence
  Synthetic
ILAKFLHWL

SEQ ID NO: 41 - protein, 9 aa, Artificial Sequence
  Synthetic
MSLQRQFLR

SEQ ID NO: 42 - protein, 9 aa, Artificial Sequence
  Synthetic
LLGPGRPYR

SEQ ID NO: 43 - protein, 9 aa, Artificial Sequence
  Synthetic
SVYDFFVWL

SEQ ID NO: 44 - protein, 9 aa, Artificial Sequence
  Synthetic
TLDSQVMSL

SEQ ID NO: 45 - protein, 10 aa, Artificial Sequence
  Synthetic
EVISCKLIKR

SEQ ID NO: 46 - protein, 9 aa, Artificial Sequence
  Synthetic
KCDICTDEY

SEQ ID NO: 47 - protein, 10 aa, Artificial Sequence
  Synthetic
RGPGRAFVTI

SEQ ID NO: 48 - protein, 8 aa, Artificial Sequence
  Synthetic
SIINFEKL

SEQ ID NO: 49 - DNA, 225 bp, Artificial Sequence
  Synthetic
gagccctcgagctccactgtctccaacatggcgaccgttgctgttctggttgtccttggagctgcaatagtcactggagctgtggtggcttttgtgatgaagatgagaaggagaaacacaggtggaaaaggaggggactatgctctggctccaggctcccagacctctgatctgtctctcccagattgtaaagtgatggttcatgaccctcattctctagcgtga

SEQ ID NO: 50 - protein, 13 aa, Artificial Sequence
  Synthetic
LRWEPSSSTVSNM

SEQ ID NO: 51 - DNA, 39 bp, Artificial Sequence
  Synthetic
ctgagatgggagccctcgagctccactgtctccaacatg

SEQ ID NO: 52 - DNA, 30 bp, Artificial Sequence
  Synthetic
ccctcgagctccactgtctccaacatggcg

SEQ ID NO: 53 - DNA, 29 bp, Artificial Sequence
  Synthetic
cgcgcggccgcaagtccactccaggcagc

SEQ ID NO: 54 - DNA, 33 bp, Artificial Sequence
  Synthetic
gcgtctagagcttcagtcgtcagcatggctcgc

SEQ ID NO: 55 - DNA, 60 bp, Artificial Sequence
  Synthetic
ctgaccggcttgtatgctgtgtatgacttttttgtgtggctcggaggtggcggatccgcg

SEQ ID NO: 56 - DNA, 60 bp, Artificial Sequence
  Synthetic
cgcggatccgccacctccgagccacacaaaaaagtcatacacagcatacaagccggtcag

SEQ ID NO: 57 - DNA, 60 bp, Artificial Sequence
  Synthetic
ctgaccggcttgtatgctagtataatcaactttgaaaaactgggaggtggcggatccgcg

SEQ ID NO: 58 - DNA, 60 bp, Artificial Sequence
  Synthetic
cgcggatccgccacctcccagtttttcaaagttgattatactagcatacaagccggtcag

SEQ ID NO: 59 - DNA, 348 bp, Artificial Sequence
  Synthetic
ggatccggaggtggttctggtggaggttcgatccagaaaacccctcaaattcaagtatactcacgccacccaccggagaatgggaagccgaacatactgaactgctacgtaacacagttccacccgcctcacattgaaatccaaatgctgaagaacgggaaaaaaattcctaaagtagagatgtcagatatgtccttcagcaaggactggtctttctatatcctggctcacactgaattcacccccactgagactgatacatacgcctgcagagttaagcatgacagtatggccgagcccaagaccgtctactgggatcgagacatgctgagatgggagccctcgagc

SEQ ID NO: 60 - DNA, 55 bp, Artificial Sequence
  Synthetic
gcgggatccggaggtggttctggtggaggttcgatccagaaaacccctcaaattc

SEQ ID NO: 61 - DNA, 45 bp, Artificial Sequence
  Synthetic
gcggctcgagggctcccatctcagcatgtctcgatcccagtagac

SEQ ID NO: 62 - DNA, 291 bp, Artificial Sequence
  Synthetic
gccctgctggtcattcctgtcgtgatgggcatcctcatcaccattttcggggtgtttctctatatcaaaaaggtggtcaagaaaccaaaggataatgagatgttaccccctgcggctcgacggcaagatccccaggagatggaagattatcccggtcataacaccgctgctccagtgcaggagacactgcacgggtgtcagcctgtcacacaggaggatggtaaagagagtcgcatctcagtgcaggagcggcaggtgacagacagcatagccttgaggcccctggtctga

SEQ ID NO: 63 - DNA, 46 bp, Artificial Sequence
  Synthetic
ccctcgagctccactgtctccaacatggccctgctggtcattcctg

SEQ ID NO: 64 - DNA, 29 bp, Artificial Sequence
  Synthetic
cgcgcggccgcggtcagcaagcagccatc

SEQ ID NO: 65 - protein, 9 aa, Artificial
  Synthetic
SLNGTTMTL

SEQ ID NO: 66 - protein, 9 aa, Artificial
  Synthetic
TLPATTLTL

SEQ ID NO: 67 - protein, 9 aa, Artificial
  Synthetic
DLAPSKGTV

SEQ ID NO: 68 - protein, 9 aa, Artificial
  Synthetic
TLSGHYATI

SEQ ID NO: 69 - protein, 9 aa, Artificial
  Synthetic
TITCLVVDL

SEQ ID NO: 70 - protein, 9 aa, Artificial
  Synthetic
YATISLLTV

SEQ ID NO: 71 - protein, 9 aa, Artificial
  Synthetic
ELASTQSEL

SEQ ID NO: 72 - protein, 9 aa, Artificial
  Synthetic
GTLTVTSTL

SEQ ID NO: 73 - protein, 9 aa, Artificial
  Synthetic
ALMRSTTKT

SEQ ID NO: 74 - protein, 9 aa, Artificial
  Synthetic
NIPSNATSV

SEQ ID NO: 75 - protein, 9 aa, Artificial
  Synthetic
ATSVTLGCL

SEQ ID NO: 76 - protein, 9 aa, Artificial
  Synthetic
TMTLPATTL

SEQ ID NO: 77 - protein, 9 aa, Artificial
  Synthetic
FTPPTVKIL

SEQ ID NO: 78 - protein, 9 aa, Artificial
  Synthetic
SVQWLHNEV

SEQ ID NO: 79 - protein, 9 aa, Artificial
  Synthetic
FICRAVHEA

SEQ ID NO: 80 - DNA, 60 bp, Artificial
  Synthetic
cgcggatccgccacctcccggctgggctgtgttacactcaaaagcatacaagccggtcag

SEQ ID NO: 81 - protein, 8 aa, Artificial
  Synthetic
FEQNTAQP

SEQ ID NO: 82 - DNA, 51 bp, Artificial
  Synthetic
ggcggatccggaggtggttctggtggaggttcgatccagaaaacccctcaa

SEQ ID NO: 83 - DNA, 42 bp, Artificial
  Synthetic
aagaccgtctactgggatcgagacatgctgagatgggagccc

SEQ ID NO: 84 - DNA, 33 bp, Artificial
  Synthetic
ccgtcgaccacctgttatatgtacaagacaatc

SEQ ID NO: 85 - DNA, 29 bp, Artificial
  Synthetic
cgcgcggccgcactgggtttaggccccag

SEQ ID NO: 86 - DNA, 30 bp, Artificial
  Synthetic
ccctcgagcacctgtcagatgaataagacc

SEQ ID NO: 87 - DNA, 35 bp, Artificial
  Synthetic
cgcgcggccgctgggcaagaaatgcctcaggaggt

SEQ ID NO: 88 - DNA, 27 bp, Artificial
  Synthetic
ccctcgagcgcactggtgtctggagtc

SEQ ID NO: 89 - DNA, 29 bp, Artificial
  Synthetic
cgcgcggccgcaggaagtcaggaactggg